(19) United States
(12) Patent Application Publication
(10) Pub. No.: US 2017/0035691 A1
(43) Pub. Date: Feb. 9, 2017

(54) FOOD MATRICES AND METHODS OF MAKING AND USING

(71) Applicants: David J. MCCLEMENTS, Amherst, MA (US); Hang XIAO, Amherst, MA (US); UNIVERSITY OF MASSACHUSETTS, Amherst, MA (US)

(72) Inventors: David J. McClements, Northampton, MA (US); Hang Xiao, Amherst, MA (US)

(21) Appl. No.: 15/302,615

(22) PCT Filed: Apr. 8, 2015
(86) PCT No.: PCT/US15/24983

§ 371 Date: Oct. 7, 2016

Related U.S. Application Data
(60) Provisional application No. 61/976,811, filed on Apr. 8, 2014.

(51) Int. Cl. A61K 9/00; A23L 33/12; A23L 33/10; A61K 31/015; A61K 31/12; A61K 31/202; A61K 47/14; A61K 47/44; A61K 9/107; A23L 33/105; A61K 31/122

(52) U.S. Cl. CPC A61K 9/0053; A23L 33/105; A23L 33/12; A23L 33/10; A61K 31/015; A61K 31/122; A61K 31/202; A61K 47/14; A61K 47/44; A61K 9/1075; A61K 31/12; A23V 2002/00

(57) ABSTRACT

A method of these teachings for designing foods in order to improve the bioavailability of orally administered bioactive agents, the method including designing a food matrix, the food matrix not having bioactivity above its normal nutritional function, that increases bioavailability of a predetermined pharmaceutical or nutraceutical by at least one of facilitating the release and solubilization of bioactive agents in the predetermined pharmaceutical or nutraceutical, altering the absorption of lipophilic bioactive agents in the predetermined pharmaceutical or nutraceutical when co-ingested, or interfering with chemical transformations that occur within gastrointestinal tract (GIT) or after absorption; the food matrix being co-ingested with the predetermined pharmaceutical or nutraceutical or ingested at a specified time soon before or after the pharmaceutical or nutraceutical.

### BACKGROUND

These teachings relate generally to improving the oral bioavailability of pharmaceuticals and nutraceuticals.

Many bioactive agents present in foods (nutraceuticals) or drugs (pharmaceuticals) intended for oral ingestion have low and/or variable bioavailability. The poor bioavailability characteristics of these bioactive agents may be the result of a number of physicochemical or physiological processes: restricted release from the product matrix; low solubility in gastrointestinal fluids; low permeability across intestinal epithelial cells; and/or, enzymatic or chemical transformations within the gastrointestinal tract (GIT). Research in the food, nutrition, and pharmaceutical disciplines has established that the bioavailability of many bioactive agents depends strongly on the nature of the foods ingested with them. Both the composition and the structural organization of the food matrix may influence the bioavailability of co-ingested bioactive agents. The dependence of the oral bioavailability of bioactive agents on food matrix characteristics means there is considerable opportunity for designing food-based delivery systems to improve the efficacy of these types of pharmaceuticals and nutraceuticals.

There is increasing convergence in the interests of the pharmaceutical and food industries in the development of products to prevent or treat human diseases. The pharmaceutical industry is developing drug preparations to combat chronic or acute diseases, whereas the food industry is developing food and beverage products whose purpose is to promote human health and wellbeing through diet. In particular, there is a considerable overlap in the development of food-based approaches to improve the oral bioavailability of bioactive agents, such as nutraceuticals and pharmaceuticals. These approaches are based on the design of the composition or structure of food matrices to increase bioavailability and have led to new classification of foods: functional foods; medical foods.

A functional food is fabricated from generally recognized as safe (GRAS) food ingredients, and typically contains one or more food-grade bioactive agent (“nutraceuticals”) dispersed within a food matrix. There are already many examples of functional food products that are commercially available, including milks fortified with vitamin D, yogurts fortified with probiotics, spreads fortified with phytosterols, and breakfast cereals fortified with ω-3 fatty acids, vitamins, and minerals. A great deal of research is currently being carried out on identifying other kinds of nutraceuticals, and it will be important for the food industry to clearly demonstrate their health benefits before they can be successfully incorporated into functional food products and obtain regulatory and consumer acceptance.

A medical food contains one or more pharmaceutical-grade bioactive agents (drugs) dispersed within a food matrix. This food matrix may be a traditional food type (such as a beverage, yogurt, or confectionary) or it may be a nutritional fluid that is fed to a patient through a tube. A medical food is usually administered to treat a particular disease under medical supervision. A number of medical foods are commercially available that are specifically designed to manage or treat various diseases, such as Alzheimer's, diarrhea, depression, diabetes, and osteoporosis.

There is a need for foods designed to improve the bioavailability of orally administered bioactive agents.

### BRIEF SUMMARY

Methods for designing foods in order to improve the bioavailability of orally administered bioactive agents are disclosed herein below.

In one or more embodiments, the method of these teachings for designing foods in order to improve the bioavailability of orally administered bioactive agents includes designing a food matrix, the food matrix not having bioactivity above its normal nutritional function, that increases the bioavailability of a predetermined pharmaceutical or nutraceutical by at least one of facilitating the release and solubilization of bioactive agents in the predetermined pharmaceutical or nutraceutical, altering the absorption of lipophilic bioactive agents in the predetermined pharmaceutical or nutraceutical when co-ingested, or interfering with chemical transformations that occur within the gastrointestinal tract (GIT) or after absorption; the food matrix being co-ingested with the predetermined pharmaceutical or nutraceutical or ingested at a specified time soon before or after the pharmaceutical or nutraceutical.

In one or more instances, the method of these teachings also includes using an in vitro GIT model or animal feeding study to verify improvement of oral bioavailability when the predetermined pharmaceutical or nutraceutical is ingested with the food matrix.

For a better understanding of the present teachings, together with other and further needs thereof, reference is made to the accompanying drawings and detailed description.

### BRIEF DESCRIPTION OF THE DRAWINGS

FIG. 1 is a diagram illustrating the convergence between the interests of the food and pharmaceutical industries;

FIG. 2 is a Schematic diagram of the difference between functional, medical, and excipient foods;

FIG. 3 is a schematic diagram showing factors affecting the oral bioavailability of a lipophilic bioactive agent;

FIG. 3a is a block diagram representation of one embodiment of the method of these teachings;

FIG. 4 is Schematic diagram of the physicochemical and physiological conditions in different regions of the human gastrointestinal tract that determine the liberation, absorption, metabolism and distribution of bioactives;

FIG. 5 is a schematic diagram showing how Bioactives in molecular form or trapped within small particles may penetrate through the mucus layer and be absorbed by epithelium cells by various mechanisms;

FIG. 6 is a bar graph showing the influence of mixing β-carotene crystals with either an oil-in-water emulsion (containing 4% corn oil) or a buffer solution (PBS) containing no oil on the bioaccessibility of β-carotene using an in vitro digestion model;

FIG. 7a is a bar graph showing the influence of carrier oil type and in vitro digestion on the mean diameter of the particles in oil-in-water nanoemulsions subjected to a simulated gastrointestinal model. The oil phase contained different ratios of a digestible oil (corn oil) and indigestible oil (lemon oil);

FIG. 7b is a bar graph showing the influence of carrier oil type and in vitro digestion on the charge (ζ-potential) of the particles in oil-in-water emulsions subjected to a simulated gastrointestinal model. The oil phase contained different ratios of a digestible oil (corn oil) and indigestible oil (lemon oil);

FIG. 8a is a graph showing the influence of in vitro digestion on the particle size distribution of oil-in-water emulsions subjected to a simulated gastrointestinal model. The oil phase initially contained 100% of digestible oil (corn oil);

FIG. 8b is a graph showing the influence of in vitro digestion on the particle size distribution of oil-in-water emulsions subjected to a simulated gastrointestinal model. The oil phase initially contained 100% of indigestible oil (lemon oil);

FIG. 8c is a graph showing the influence of in vitro digestion on the particle size distribution of oil-in-water emulsions subjected to a simulated gastrointestinal model. The oil phase initially contained 50% indigestible oil (lemon oil) and 50% digestible oil (corn oil);

FIG. 9 is photographs showing the influence of carrier oil composition on the microstructure of emulsions measured by optical microscopy at various stages in an in vitro gastrointestinal tract model. The oil phase initially corn oil (100% digestible oil), lemon oil (100% indigestible oil) or a mixture (50% lemon oil and 50% corn oil). The scale bars are 20 μm long;

FIG. 10 is a graph showing the effect of carrier lipid on rate and extent of lipid digestion, measured using a pH-stat method after passing the emulsions through an in vitro digestion model. The oil phase contained different ratios of a digestible oil (corn oil) and indigestible oil (lemon oil) indicated as % corn oil on the right hand side;

FIG. 11 is a bar graph showing the effect of carrier lipid on bioaccessibility of β-carotene measured after centrifugation of emulsions passed through an in vitro digestion model. The oil phase contained different ratios of a digestible oil (corn oil) and indigestible oil (lemon oil);

FIG. 12 is a graph showing the relationship between the bioaccessibility of β-carotene and the amount of free fatty acids released during digestion;

FIG. 13a is a graph showing particle size distribution of the “micelle” phase collected after digestion and centrifugation. Measurement were made after filtration. Particle concentration is shown as intensity-weighted values;

FIG. 13b is a graph showing particle size distribution of the “micelle” phase collected after digestion and centrifugation. Measurement were made after filtration. Particle concentration is shown as volume-weighted values;

FIG. 14a is a photograph showing the appearance of raw digesta after centrifugation. From left to right the corn oil percentages in oil phase decreased from 100% to 0%;

FIG. 14b is a bar graph showing the influence of carrier oil type on the particle size and visual appearance of micelle phase collected after digestion and centrifugation. Measurements were made after filtration;

FIG. 15a is a graph showing the volume of alkaline solution titrated into the in vitro digestion medium to keep the pH constant during the course of the simulated small intestine stage;

FIG. 15b is a graph showing calculated free fatty acids released from the corn oil emulsions over time during the course of the simulated small intestine stage;

FIG. 16 is a bar graph showing the influence of droplet size and composition on concentration of heptadecanoic acid in duodenum, jejunum, and ileum of rats after feeding. Data points represent means (n=3)±standard deviations. For each group, different letters on the top of the columns represent significant differences (P<0.05). Different capital letters (A,B) mean statistical differences in the heptadecanoic acid concentration in the duodenum, jejunum or ileum in samples of a given droplet size (i.e., the effect of intestinal region). Different lower case letters (a,b) mean statistical differences in the heptadecanoic acid concentration in particular small intestine regions in samples with different droplet sizes (i.e., the effect of droplet size);

FIG. 17 is a bar graph showing the influence of droplet size and composition on concentration of Coenzyme Q10 in duodenum, jejunum, and ileum of rats after feeding. Data points represent means (n=3)±standard deviations. For each group, different letters on the top of columns represent significant differences (P<0.05). Different capital letters mean statistical differences in the CoQ10 concentration in the duodenum, jejunum or ileum in samples of a given droplet size (i.e., the effect of intestinal region). Different lower case letters mean statistical differences in the CoQ10 concentration in particular small intestine regions in samples with different droplet sizes (i.e., the effect of droplet size);

FIG. 18a is a graph showing particle size distributions of mixtures of curcumin and excipient emulsion after incubation at 30° C. for different times;

FIG. 18b is a graph showing particle size distributions of mixtures of curcumin and excipient emulsion after incubation at 100° C. for different times;

FIG. 18c is photographs of mixtures of curcumin and excipient emulsion after incubation at 30 and 100° C. Note: yellow sediment (curcumin crystals) was observed at the bottom of the test tubes held at 30° C., whereas a yellow oil layer was observed at the top of the test tubes after heating at 100° C. for 60 minutes (red arrows);

FIG. 19a is photographs showing the effect of temperature on microstructure of mixtures of curcumin and excipient emulsion;

FIG. 19b is photographs showing the effect of temperature on polarized light microscopy of curcumin and excipient emulsion;

FIG. 20a is a graph showing the absorbance versus temperature profile of curcumin-corn oil mixtures (3 and 4 mg/mL);

FIG. 20b is a graph showing the absorbance versus time profile of a curcumin-corn oil mixture (3 mg/mL) at different isothermal storage temperatures;

FIG. 21a is a bar graph showing the influence of simulated gastrointestinal conditions on the mean droplet diameter (d₃₂) of curcumin-emulsion and curcumin-oil mixtures after incubation at 30° C. for 30 min or at 100° C. for 10 min. Samples designated with different letters (a, b, c) were significantly different (Duncan, p<0.05);

FIG. 21b is a photographic image of micelle phase collected from curcumin-emulsion and curcumin-oil mixtures;

FIG. 22a is a graph showing the influence of simulated gastrointestinal conditions on the particle size distributions of curcumin-emulsion mixture after 30 min incubation at 30° C.;

FIG. 22b is a graph showing the influence of simulated gastrointestinal conditions on the particle size distributions of curcumin-emulsion mixture after 10 min incubation at 100° C.;

FIG. 22c is a graph showing the influence of simulated gastrointestinal conditions on the particle size distributions of curcumin-oil mixture in the small intestine (measurements could not be made in the initial, mouth, or stomach phases for this sample);

FIG. 23 is photographs showing the influence of simulated gastrointestinal conditions on microstructure of curcumin-emulsion and curcumin-oil mixtures exposed to different incubation conditions (30° C. for 30 min or 100° C. for 10 min) determined by confocal fluorescence microscopy. The scale bars represent a length of 20 m, and the red regions represent lipids;

FIG. 24 is a bar graph showing the influence of simulated gastrointestinal conditions on the particle charge of curcumin-emulsion and curcumin-oil mixtures exposed to different incubation conditions (30° C. for 30 min or 100° C. for 10 min). Samples designated with different letters (a, b, c) were significantly different (Duncan, p<0.05);

FIG. 25 is a graph showing the influence of incubation temperature on the free fatty acids (FFA %) release profile for curcumin-emulsion and curcumin-oil mixtures exposed to different incubation conditions (30° C. for 30 min or 100° C. for 10 min);

FIG. 26 is a schematic diagram of the pre-ingestion and post-ingestion solubilization of curcumin in excipient emulsions. Prior to ingestion, curcumin may be solubilized in oil droplets when powdered curcumin is incubated with the emulsions. After ingestion, curcumin may be solubilized within the mixed micelles resulting from digestion of the oil droplets;

FIG. 27a is a graph showing particle size distributions of mixtures of curcumin and excipient emulsion (small, medium and large blank emulsion) after incubation at 30° C. for 30 min and 100° C. for 10 min;

FIG. 27b is photographs of mixtures of curcumin and excipient emulsion (small, medium and large blank emulsion) after incubation at 30 and 100° C.;

FIG. 28a is photographs of the effect of temperature on microstructure of mixtures of curcumin and excipient emulsion in three different particle sizes;

FIG. 28b is photographs of the effect of temperature on polarized light microscopy of curcumin and excipient emulsion in three different particle sizes;

FIG. 29 is a bar graph of the influence of simulated gastrointestinal conditions on the mean droplet diameter (d₃₂) of curcumin-emulsion with different particle size mixtures after incubation 100° C. for 10 min. Different lowercase letters mean significant differences (p<0.05) of the droplet diameter of an emulsion between digestion phases; Different capital letters mean significant differences (p<0.05) of the droplet diameter between emulsion types (Small, Medium and Large) within the same digestion phase;

FIG. 30a is a graph of the influence of simulated gastrointestinal conditions on the particle size distributions of: (a) curcumin—small emulsion mixture after 10 min incubation at 100° C.;

FIG. 30b is a graph showing the influence of simulated gastrointestinal conditions on the particle size distributions of: (b) curcumin—medium emulsion mixture after 10 min incubation at 100° C.;

FIG. 30c is a graph showing the influence of simulated gastrointestinal conditions on the particle size distributions of: (c) curcumin—large emulsion mixture after 10 min incubation at 100° C.;

FIG. 31 is photographs showing the influence of simulated gastrointestinal conditions on microstructure of curcumin-emulsion with different particle size mixtures after 100° C. for 10 min incubation determined by confocal fluorescence microscopy. The scale bars represent a length of 20 μm, and the red regions represent lipids;

FIG. 32 is a bar graph showing the influence of simulated gastrointestinal conditions on the particle charge of curcumin-emulsion with different particle size after 100° C. for 10 min incubation. Samples designated with different letters (a, b, c) were significantly different (Duncan, p<0.05);

FIG. 33 is a graph showing the influence of incubation temperature on the free fatty acids release profile for curcumin-emulsion with different particle size and emulsifier mixtures after 100° C. for 10 min incubation;

FIG. 34 is a bar graph showing the influence of particle size on the total curcumin concentration in the raw digesta (C_{Raw digesta}) and micelle phase (C_{Micelle}), as well as on curcumin bioaccessibility (BA). Samples designated with different letters (a and b) were significantly different (Duncan, p<0.05);

FIG. 35 is Table 4 of the specification;

FIG. 36 is Table 5 of the specification;

FIG. 37 is Table 6 of the specification;

FIG. 38 is Table 7 of the specification; and

FIG. 39 is Table 8 of the specification.

### DETAILED DESCRIPTION

The following detailed description presents the currently contemplated modes of carrying out the invention. The description is not to be taken in a limiting sense, but is made merely for the purpose of illustrating the general principles of the invention, since the scope of the invention is best defined by the appended claims.

As used herein, the singular forms “a,” “an,” and “the” include the plural reference unless the context clearly dictates otherwise.

Except where otherwise indicated, all numbers expressing quantities of ingredients, reaction conditions, and so forth used in the specification and claims are to be understood as being modified in all instances by the term “about.”

Methods for designing foods in order to improve the bioavailability of orally administered bioactive agents are disclosed herein below.

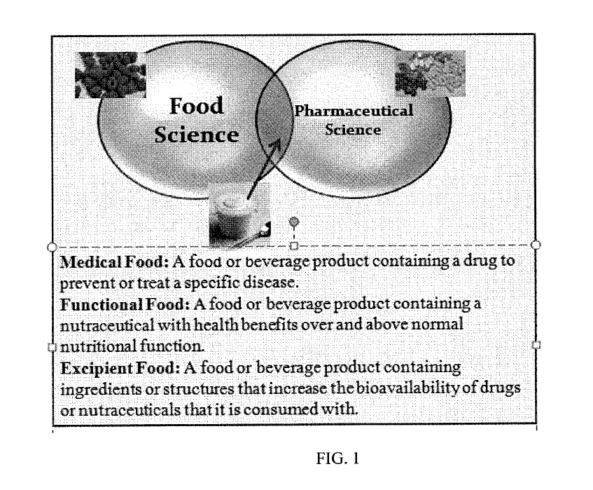

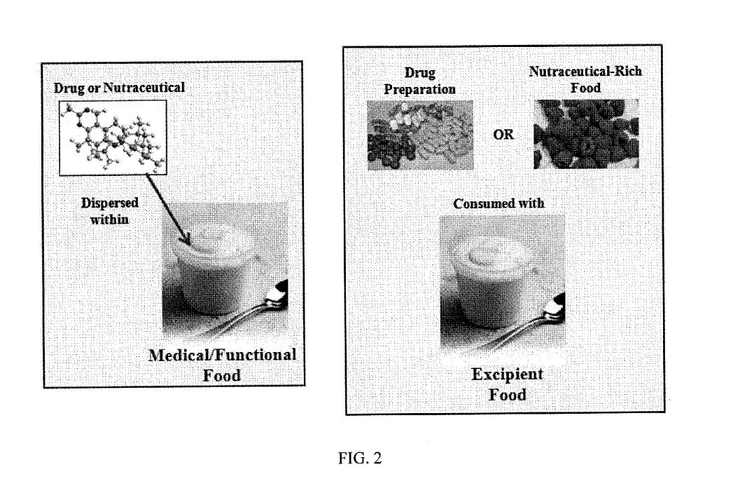

Hereinbelow, a class of foods designed to improve the bioavailability of orally administered bioactive agents is introduced: excipient foods (see FIGS. 1, 2). An excipient is conventionally defined as a component that is not bioactive itself but is included in a pharmaceutical preparation to increase the efficacy of a drug. Some commonly used excipients in the pharmaceutical industry include lipids, surfactants, synthetic polymers, carbohydrates, proteins, cosolvents, and salts. By analogy, an excipient food may not have any bioactivity itself (above its normal nutritional attributes), but it may increase the efficacy of any nutraceuticals or pharmaceuticals that are co-ingested with it. Excipient foods are therefore meant to be consumed with a conventional pharmaceutical dosage form (e.g., capsule, pill, or syrup), a dietary supplement (e.g., capsule, pill, or syrup), or nutraceutical-rich food (e.g., fruits, vegetables, nuts, seeds, grains, meat, fish, and some processed foods). It is likely that different kinds of excipient foods will have to be designed for different types of bioactive agents or delivery matrices. Some examples of excipient foods that could be developed to increase the bioavailability of nutraceuticals in foods are shown in Table 1. For example, the bioaccessibility of carotenoids in a salad may be increased by consuming it with a specifically designed salad dressing. This dressing may contain various food components that increase the bioavailability of the nutraceuticals in the salad: lipids that increase intestinal solubility; antioxidants that inhibit chemical transformations; enzyme inhibitors that retard metabolism; permeation enhancers that increase absorption; efflux inhibitors. Indeed, previous studies have shown that the bioavailability of oil-soluble vitamins and carotenoids in salads can be increased by consuming them with dressings containing some fat, which supports the concept of excipient foods.

TABLE 1
Examples of excipient foods that could be designed to
improve the bioactivity of nutraceuticals in foods.

| | | Potential Excipient |
|---|---|---|
| Nutraceuticals | Food Source | Foods |
| Carotenoids | Salad (lettuce, kale, | Salad Dressing |
| | carrot, tomato, peppers . . .) | |
| Carotenoids | Cooked vegetables | Sauce |
| | (carrot, peppers, spinach, | |
| | kale . . .) | |
| Carotenoids, | Nuts and Seeds | Edible Coatings |
| Vitamins, | (almonds, peanuts, sunflower | |
| Phytosterols/stanols | seeds . . .) | |
| Flavonoids, | Fruits and Berries | Cream, Ice Cream, |
| Vitamins | (blueberry, strawberry, | Yogurt. |
| | raspberry, apple, pear . . .) | |
| Phytosterols/stanols | Nuts | Sauce, Edible |
| | | Coatings |
| CLA | Meat and dairy products | Sauce |
| | (beef, cheese, . . .) | |
| ω-3 Oils | Fish | Sauce |

In one or more embodiments, the method of these teachings for designing foods in order to improve the bioavailability of orally administered bioactive agents includes designing a food matrix, the food matrix not having bioactivity above its normal nutritional function, that increases bioavailability of a predetermined pharmaceutical or nutraceutical by at least one of facilitating the release and solubilization of bioactive agents in the predetermined pharmaceutical or nutraceutical, altering the absorption of lipophilic bioactive agents in the predetermined pharmaceutical or nutraceutical when co-ingested, or interfering with chemical transformations that occur within the gastrointestinal tract (GIT) or after absorption; the food matrix being co-ingested with the predetermined pharmaceutical or nutraceutical or ingested at a specified time soon before or after the pharmaceutical or nutraceutical.

In one instance, in one embodiment of the method of these teachings, the facilitating the release and solubilization of bioactive agents in the predetermined pharmaceutical or nutraceutical comprises at least one of enhancing breakdown of a matrix surrounding a bioactive agent, enhancing solubilization with a mixed micelle phase, altering mass transport processes within the GIT, or altering the motility of the GIT.

In another instance, in one embodiment of the method of these teachings, the altering the absorption of lipophilic bioactive agents in the predetermined pharmaceutical or nutraceutical when co-ingested comprises at least one of increasing transport across a layer of epithelial cells surrounding the GIT or inhibiting the efflux mechanisms in membranes of intestinal epithelial cells.

In principle, a wide variety of different food products could be used as excipients to increase the bioactivity of lipophilic bioactives, such as beverages, yogurts, dressings, desserts, sauces, soups, dips, spreads, candies, and baked goods. These excipient foods need to be selected so that they are economic, convenient, desirable, and effective, and that can be regularly incorporated into a daily diet.

In one or more embodiments, the method of these teachings for designing foods in order to improve the bioavailability of orally administered bioactive agents includes designing a food matrix, the food matrix not having bioactivity above its normal nutritional function, that increases bioavailability of a predetermined pharmaceutical or nutraceutical by at least one of facilitating the release and solubilization of bioactive agents in the predetermined pharmaceutical or nutraceutical, altering the absorption of lipophilic bioactive agents in the predetermined pharmaceutical or nutraceutical when co-ingested, or interfering with chemical transformations that occur within gastrointestinal tract (GIT) or after absorption; the food matrix being co-ingested with the predetermined pharmaceutical or nutraceutical or ingested at a specified time soon before or after the pharmaceutical or nutraceutical.

Hereinbelow, the design of excipient foods is considered, then the main factors limiting the bioavailability of lipophilic bioactive components are highlighted, and then the impact of food matrix composition and structure on bioavailability and how this leads to the concept of excipient foods and excipient food ingredients is discussed.

Excipient foods may be fluids, semi-solids, or solids that may be consumed by drinking (beverages) or eating (foods). A number of different factors must be considered when designing excipient foods. First, the composition and structure of the food matrix should be designed to increase the bioavailability of co-ingested bioactive agents. This depends on knowledge of the influence of specific food components and structures on the biological fate of the bioactives. Second, the food matrix should be designed so that the product is desirable to consumers or patients to ensure good compliance, e.g., the food should have a desirable appearance, texture, mouthfeel, and flavor. Third, foods or beverages should be chosen so that they can be consumed on a regular basis with drugs or foods containing nutraceutical agents (such as fruits and vegetables). This restricts the type of products suitable for use as excipient foods to those that can easily be incorporated into a daily diet. Fourth, the product should have a sufficiently long shelf life and not take up too much storage space, since it is impractical for consumers to purchase a product too frequently. Some potential candidates for excipient foods that meet most or all of these requirements are fruit drinks, teas, coffees, dairy beverages, creams, yogurts, margarine, butter, cheese spreads, desserts, confectionary, and crackers. The nature of the excipient food might depend on the type of drug or nutraceutical-rich food that is being consumed. For example, an excipient food suitable for increasing the bioavailability of the nutraceuticals in fruits (such as apples, pears, blueberries, strawberries, or raspberries) might consist of a specially designed cream, yogurt, or ice cream. On the other hand, an excipient food suitable for increasing the bioavailability of nutraceuticals in cooked or raw vegetables (such as carrots, broccoli, spinach, or kale) might consist of a specially designed pouring sauce or salad dressing.

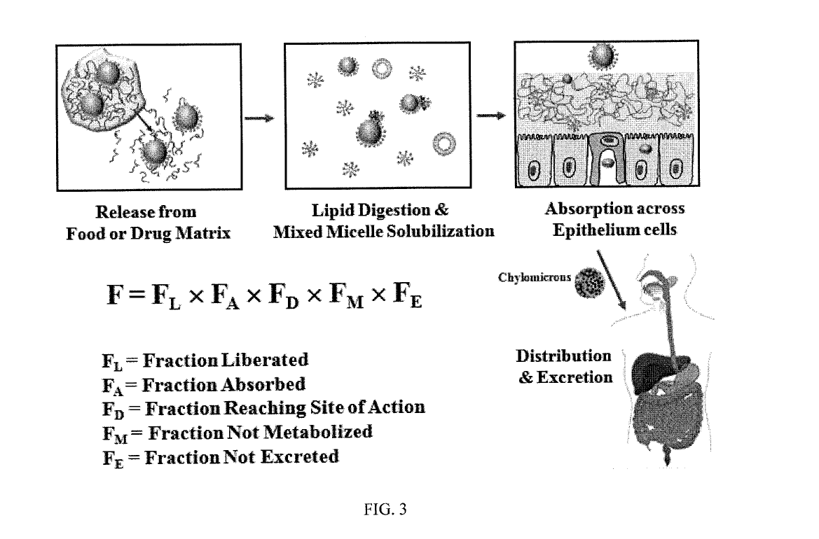

It is useful to highlight the major factors limiting the bioavailability of lipophilic bioactive agents since this information will aid in the successful development of efficacious excipient foods. The oral bioavailability of an ingested bioactive component depends on the fraction that reaches the target site-of-action in a biologically active form. The overall bioavailability (F) of a lipophilic bioactive component depends on numerous factors (FIG. 3):

F=F_{L}×F_{A}×F_{D}×F_{M}×F_{E} (1)

F_{L} is the fraction of bioactive agent liberated from its original environment, which may be a drug preparation or a food matrix, into the GIT so that it becomes bioaccessible i.e., in a form suitable for absorption (F_{L}). F_{A} is that fraction of the liberated bioactive agent that is absorbed by the epithelial cells within the GIT. F_{D} is the fraction of absorbed bioactive agent that reaches the site of action after distribution amongst the various tissues of the body e.g., blood, liver, kidney, heart, brain, muscles, adipose tissue etc. F_{M} is the fraction of bioactive component that reaches the site of action in a metabolically active form, which depends on any chemical or enzymatic transformations that take place after ingestion e.g., hydrolysis, oxidation, and conjugation. F_{E} is the fraction of metabolically active bioactive component that remains at the site of action, i.e., has not been excreted. In reality, each of these parameters varies over time after a bioactive agent has been ingested to give a profile of bioavailability (F) versus time (t) at a specified site of action. Typically, the overall bioavailability increases sometime after ingestion, and then decreases as the bioactive agent is metabolized, stored, utilized, distributed, or excreted. Ultimately, the bioactivity of an ingested bioactive component depends on how its bioavailability changes over time in the target tissue. A number of physiological and physicochemical factors that influence the bioavailability of lipophilic bioactive components have been established, and are summarized in the following sections.

Liberation

A lipophilic bioactive agent must be liberated from a food matrix (e.g., fruit, vegetable, fish, meat, processed food) or drug preparation (e.g., pill or capsule) and then solubilized within mixed micelles in the small intestinal fluids before it becomes accessible for absorption (FIG. 3). Mixed micelles are assembled from bile salts and phospholipids secreted by the body, as well as any lipid digestion products such as monoacylglycerols and free fatty acids. It should be stressed that the expression “mixed micelles” actually refers to a compositionally, structurally, and dynamically complex mixture within the GIT that may contain various colloidal structures, such as micelles, vesicles, and liquid crystals that changes over time during the digestion and absorption processes. The fraction of an ingested lipophilic bioactive agent that is solubilized within the mixed micelle phase of the small intestine is usually taken to be a measure of the fraction that is liberated (F_{L}) in a form suitable for absorption.

Absorption

Mixed micelles are able to transport solubilized lipophilic bioactive agents through the mucus layer and to the apical side of the intestinal epithelial cells (FIG. 3). The bioactives may then be incorporated into the epithelial cells through various passive or active transfer mechanisms. At present, it is not clear whether the bioactive agents are first released from the mixed micelles into the surrounding aqueous phase and then absorbed, or whether they are absorbed as part of the mixed micelles e.g., by fusion with the cell membranes. In addition, it is also possible for bioactive molecules trapped within other types of colloidal particles (such as engineered nanoparticles) to be directly absorbed by intestinal epithelial cells. Overall, the fraction of the bioactive agent that is transported into the epithelial cells is usually taken as a measure of the fraction absorbed (F_{A}) by the body.

Metabolism

After ingestion, lipophilic bioactive agents may be transformed as they pass through the GIT or after they have been absorbed due to various chemical processes (such as acid hydrolysis or lipid oxidation) or biochemical processes (such as digestive or metabolic enzyme activity). The presence of digestive enzymes (such as lipases and phospholipases) may catalyze the breakdown of some lipophilic bioactive agents (such as triacylglycerols, phospholipids or Vitamin E acetate). The presence of metabolic enzymes changes the chemical structures of some ingested lipophilic bioactive agents, thereby altering their physicochemical and physiological characteristics. The extent of metabolism often depends on the route that the bioactive agents are transported into the systemic circulation. Strongly hydrophobic agents tend to be transported via the lymphatic route, whereas less hydrophobic agents tend to be transported via the portal vein and liver. Lipophilic bioactives may be highly metabolized when they pass through the liver before reaching the systemic circulation, thereby altering their biological activity. In some cases, molecular transformations increase bioactivity, whereas in other cases they decrease it. The transformation of a lipophilic bioactive as it travels through the GIT and human body determine the fraction that arrives at the site of action in a metabolically active state (F_{M}). The ability to alter the absorption pathway of bioactive agents by manipulating dietary composition or structure provides an important way of increasing the bioavailability of certain bioactives.

Distribution

After a lipophilic bioactive agent has been absorbed it is usually distributed amongst various tissues within the human body (FIG. 3), such as the systemic circulation, liver, kidney, muscles, adipose tissue, heart, lungs, brain, etc. The distribution of the bioactive agent depends on the molecular characteristics of the bioactive, as well as those of any co-ingested food components. The target tissue(s) for a bioactive agent depends on the nature of the biological response required, such as enhanced performance, maintenance of general wellbeing, prevention of chronic disease, or treatment of specific acute diseases.

Excretion

Lipophilic bioactives and there metabolites are eventually removed from the human body through a variety of mechanisms, and often end up within the feces, urine, sweat, or breath. It may therefore be possible to increase the bioavailability of an ingested bioactive by increasing its persistence within the human body. The rate of excretion determines the fraction of bioactive agent that remains at the site of action (F_{E}) at a particular time.

Improving Oral Bioavailability

The oral bioavailability of ingested lipophilic agents can be improved by designing excipient foods that increase the fraction liberated (F_{L}), absorbed (F_{A}), and reaching the site of action (F_{D}) in a metabolically active form (F_{M}). This goal can be achieved by manipulating the composition and structure of food matrices based on knowledge of the impact of specific food matrix properties on the biological fate of ingested lipophilic bioactives.

Impact of Food Matrix on Bioavailability

The oral bioavailability of lipophilic bioactives in drugs or foods may be increased by ingesting them with excipient foods with specifically designed compositions and structures. In this section, some of the major ways in which food components may alter the oral bioavailability of lipophilic bioactive agents is highlighted. It is assumed that an excipient food should be fabricated entirely from food-grade ingredients that are generally recognized as safe (GRAS). An excipient food could then be marketed and distributed as a conventional food product with additional health benefits.

Potential Mechanisms of Action

The components within an excipient food may alter the oral bioavailability of co-ingested lipophilic bioactives through various physicochemical or biochemical mechanisms as disclosed below.

Bioactive Liberation

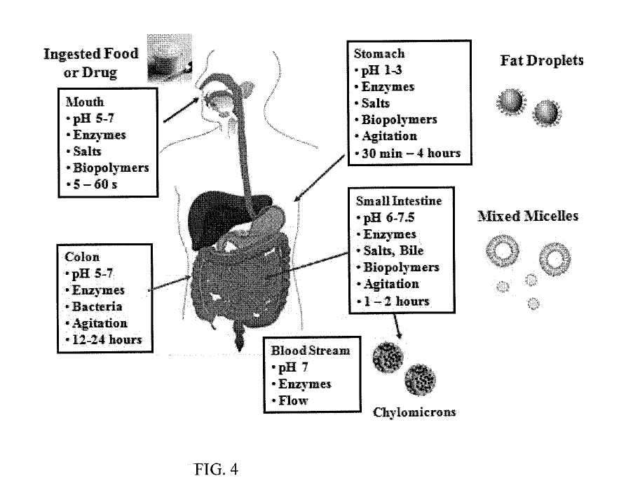

Prior to ingestion, lipophilic bioactive agents are typically trapped within some kind of fluid, semi-solid, or solid matrix in pharmaceutical or drug products. For example, a lipophilic drug may be present within a pill or capsule, whereas a lipophilic nutraceutical may be trapped inside the cells of a fruit or vegetable or within the fat droplets in a processed food. The bioactive agents must therefore be liberated from their original location before they can be solubilized within intestinal fluids and absorbed by the body (FIG. 3). An excipient food may therefore be designed so that it contains specific ingredients that facilitate the release and solubilization of bioactive agents. The design of this type of food requires knowledge of the physicochemical and physiological processes that occur within the human gastrointestinal tract after ingestion (FIG. 4).

Release from Food or Drug Matrix

The breakdown of the matrix surrounding a bioactive agent within the human GIT is usually carried out by mechanical, chemical, and enzymatic means. Foods are usually masticated within the mouth to break them down into smaller fragments prior to swallowing, whereas pharmaceutical preparations (such as capsules and pills) are usually swallowed directly. After swallowing, pharmaceutical or drug matrices may be broken down in the stomach and small intestine due to the mechanical motions of the GIT, e.g., peristalsis or grinding. The high acidity and ionic strength of the stomach also facilitates the dissociation of certain structures, particularly those held together by electrostatic interactions. Some matrix dissociation may also occur due to the simple fact that the material is dissolved within an aqueous environment, e.g., pills, capsules, or powders formed from water-soluble substances such as carbohydrates or proteins. The activity of digestive enzymes (such as amylases, proteases, and lipases) stimulates the breakdown of major food components (such as starches, proteins, and lipids), which often play an important role in maintaining the matrix structure in foods and drug preparations. Secreted biological surfactants in the GIT, such as bile salts and phospholipids, may also facilitate the breakdown of matrix structures held together by hydrophobic interactions in foods and drug preparations, particularly those containing lipids or surface active agents.

Excipient foods may enhance one or more of these processes by numerous mechanisms. Ingestion of an excipient food may stimulate the release of hormones that promote the release of acids, enzymes, or bile salts within the GIT, thereby promoting the liberation of bioactive agents by facilitating the breakdown of matrix structures in foods or drug preparations. The co-ingestion of bioactive lipophilic agents with an excipient food may change their bioavailability by altering their transit time within the GIT. Food components that delay transit may lead to higher absorption of bioactive agents since then there is more time for them to be liberated and absorbed. The presence of fats within an excipient food may facilitate the release of lipophilic bioactive agents from co-ingested foods or pharmaceuticals by acting as an organic solvent. Salts, acids, bases, or chelating agents in an excipient food may contribute to the breakdown of matrix structures in foods or drug preparations by altering the molecular interactions between structural components. A number of food components may alter the intestinal pH due to their acidity, alkalinity or buffering capacity. For example, ingestion of high amounts of protein may lead to a higher gastric pH due to the strong buffering capacity of some protein molecules. Changes in pH may alter the rate and extent of breakdown of food or pharmaceutical matrix structures and therefore the liberation of bioactive components.

Solubilization in Mixed Micelles

After a lipophilic bioactive agent is liberated from the original food or pharmaceutical matrix it needs to be solubilized within the mixed micelle phase so that it can be transported to the intestinal epithelial cells. It is well established that co-ingestion of lipophilic drugs or nutraceuticals with lipids can greatly increase their oral bioavailability, which can be attributed to a number of factors. First, ingestion of lipids stimulates the release of digestive enzymes and bile salts, as well as increasing GIT transit time. An increase in the bile salt levels increases the solubilization capacity of the intestinal fluids, whereas as an increase in GIT transit time increases the time available for any ingested bioactive agents to be liberated, solubilized, and absorbed. Second, the digestion of co-ingested lipids (triglycerides) within the GIT leads to the formation of free fatty acids (FFA) and monoacylglycerols (MAG) that are incorporated into the mixed micelles in the small intestine thereby increasing their solubilization capacity for lipophilic bioactives (see later section). Third, ingestion of any surface active substances (such as phospholipids or surfactants) may also increase the solubilization capacity of the intestinal fluids due to their ability to be incorporated into mixed micelles.

Alteration of Mass Transport Processes

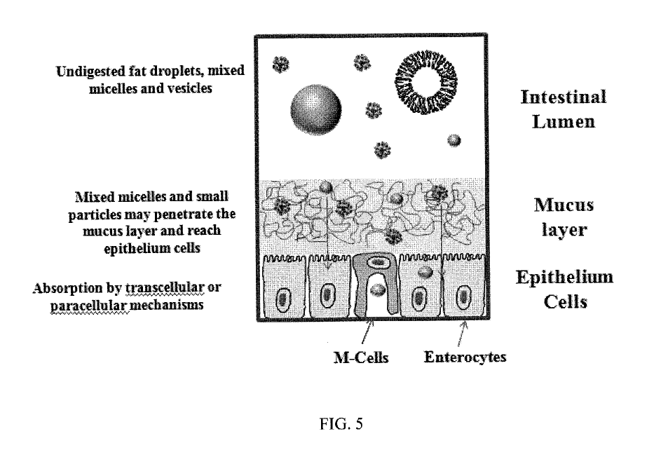

The liberation of lipophilic bioactive agents within the GIT often depends on the mass transport of reactants, catalysts, and products from one location to another. Digestive enzymes must come into close proximity to their substrates before they can carry out their catalytic actions. Bioactive agents solubilized within mixed micelles must be transported through the lumen and across the mucous layer before they can be absorbed by epithelial cells (FIG. 5). The rate and extent of liberation of bioactive agents from food or drug matrices may therefore be controlled by incorporating food ingredients within excipient foods that alter mass transport processes within the lumen of the GIT. In general, mass transport may occur by convective or diffusive processes, depending on the structural and physicochemical properties of the intestinal fluids and the flow profile within the region of the GIT involved. The mechanical forces generated by the GIT mix components together and help move them from one location to another. Nevertheless, there are regions within the GIT where mass transport is primarily diffusion-limited, e.g., the movement of small molecules through gelled phases. Excipient food components may be able to alter diffusion-limited or convection-limited processes by various mechanisms: binding to bioactive agents; altering the microscopic or macroscopic rheology of the intestinal fluids; altering GIT motility. For example, some biopolymers are able to form viscous solutions or gels under simulated gastrointestinal conditions, and may therefore be able to alter mass transport and transit times, which in turn alter important events affecting the release and processing of bioactive agents. Cationic biopolymers, such as chitosan, are able to bind anionic bile salts and free fatty acids, and therefore alter their mass transport.

Alterations in Gut Motility

Certain kinds of food components have been shown to alter the motility of the GIT, e.g., gastric emptying time or the mechanical actions of the stomach and small intestine. The co-ingestion of a bioactive agent with a meal often increases the length of time it spends within the stomach. Specific phytochemicals, such as piperine, have also been shown to inhibit gastric emptying. The longer a food spends within the stomach the greater time there is for the breakdown of any matrices that normally inhibit the liberation of the bioactive agents into the intestinal fluids (e.g., cell walls in plant tissues or solid drug forms). In addition, an increase in gastric emptying time may increase the amount of digestion, metabolism, or chemical transformation of a substance that occurs within the stomach. In some cases, this may increase the bioavailability of an ingested nutraceutical or pharmaceutical, e.g., if the transformed form has a higher bioavailability than the original form, or if some of the components released from the food matrix increase the subsequent solubilization or absorption of the bioactive agents. In other cases, an increase in gastric retention may decrease bioavailability, e.g., if the transformed form has a lower bioavailability than the original form, or if some of components released from the food matrix inhibit the subsequent solubilization or absorption of the bioactive agents. Furthermore, an increase in the gastric emptying time also slows down the rate at which bioactive agents are transported to small intestine, which may have a significant impact on their absorption and metabolism in the small intestine.

Bioactive Absorption

There are numerous physicochemical and physiological mechanisms by which food matrix components could alter the absorption of co-ingested lipophilic bioactive agents. A number of the most important mechanisms that might be used in the development of excipient foods are highlighted in this section.

Increase in Membrane Permeability

The bioavailability of some lipophilic bioactive agents is limited by their transport across the layer of epithelial cells surrounding the GIT. When bioactive agents reach the apical side of the intestinal epithelial cells they may be transported into the systemic circulation by a number of passive or active transport processes (FIG. 5). The precise mechanism(s) involved depend on the molecular characteristics of the bioactive, the nature of any particles that the bioactive might be trapped within or bound with, the composition and structure of the surrounding intestinal fluids, and the region of the GIT where absorption occurs.

The two major types of epithelial cells that line the gastrointestinal tract in regions where the majority of absorption occurs are enterocytes and M-cells. Enterocytes are the most numerous type of cell lining the GIT, and they are where most of the absorption of molecular forms of drugs and nutraceuticals occur. Enterocytes also have ability to absorb certain types of particulate matter. Conversely, M-cells are much less numerous than enterocytes, typically occupying less than 1% of the epithelium surface, but they are much more efficient than enterocytes at absorbing particulate matter. M-cells are mainly found in specialized regions on the epithelium surface referred to as “Peyers patches”, which are primarily responsible for absorbing ingested antigens, such as macromolecules, microorganisms, and certain types of particles. The absorbed particles are then transported to the underlying lymphoid system where they promote immune responses.

Molecules and particles reaching the epithelial cells may be absorbed through a number of mechanisms depending on their characteristics:

Paracellular: Small molecules and particles are able to pass through the narrow gaps (“tight junctions”) that separate neighboring epithelial cells (FIG. 5). Typically, only substances that are smaller than a few nanometers are able to pass through the tight junctions. However, some substances found in foods have been shown to be capable of increasing the dimensions of the tight junctions and may therefore be able to enhance transport by this mechanism, e.g., some surfactants, polymers, minerals, and chelating agents. Specific examples of food-grade substances that might be used to increase the permeability of epithelial cells by increasing the dimensions of the tight junctions include the surfactant Tween 80, the polymer chitosan, the mineral zinc, and the chelating agent EDTA.

Transcellular—Molecules and particles may also be transported through epithelial cell membranes by passive or active transport mechanisms (FIG. 5). Many fairly lipophilic molecules are transferred across cell membranes by a passive mechanism. After encountering the epithelial cells, they are solubilized within the non-polar phospholipid tails that make up the phospholipid bilayer of the cell membrane. After moving across the cell membrane, they are incorporated into various vesicle-like structures on the other side, which then move them into the cell interior. Other types of molecules (particularly more hydrophilic ones) are transferred across the cell membrane by membrane protein-transporter systems. The absorption of particles that are small enough to travel through the mucus layer and reach the surface of the epithelial cells typically occurs by an “endocytosis” mechanism. In this case, particles come into contact with the outer wall of the cell membrane, the membrane then wraps itself around the particle, and then part of the membrane buds-off to form a vesicle-like structure with a particle trapped inside that moves into the interior of the cell. This process may occur in enterocyte cells, but is typically much more active in M-cells. The critical cut-off particle size for endocytosis has been estimated to be from less than 50 to around 100 nm for enterocyte cells, and to be from 20 to 500 nm for M-cells.

Certain types of molecules present in foods may be able to increase the transcellular uptake of lipophilic bioactive agents by epithelial cells by altering cell membrane permeability. Piperine (a compound found in black pepper) has been shown to be capable of increasing cell membrane permeability. Food grade surfactants (sucrose monoesters) have also been shown to increase membrane permeability to model drugs. Rhamnolipids have been shown to increase both transcellular and paracelluar transport of model drugs.

Persorption: Molecules or particles may also be absorbed through temporary pores formed in the layer of epithelial cells lining the GIT due to gaps formed when some of the cells are shed and replaced.

Inhibition of Efflux Mechanisms

The bioavailability of certain types of lipophilic bioactive agents is limited due to the presence of efflux mechanisms in the membranes of the intestinal epithelial cells. After absorption by epithelial cells, some bioactives are transported back into the intestinal lumen by specific transports at the apical side of the cell membrane. For example, both P-glycoprotein (P-gp) and multidrug resistant protein (MRP) have been shown to pump out a wide range of lipophilic bioactives from epithelial cells lining the GIT. This efflux process can reduce the bioavailability of bioactive agents by two mechanisms: (i) decreasing the total amount absorbed; and, (ii) increasing the extent of metabolism within the GIT if the bioactive is pumped out and then reabsorbed, which increase exposure of the bioactive to metabolizing enzymes inside of the epithelial cells. Certain types of food-grade components have been shown to be able to block efflux mechanisms, and thereby increase the net absorption of lipophilic bioactive agents by epithelial cells, e.g., some surfactants, chelating agents, biopolymers, and phytochemicals. For example, resveratrol, quercetin and piperine have been shown to act as efflux inhibitors for certain kinds of drugs. In general, three different mechanisms have been proposed for the ability of these components to inhibit efflux processes: (i) blocking binding sites on the efflux protein surfaces; (ii) interference with ATP hydrolysis (which provides the energy needed for efflux protein action); (iii) alteration of cell membrane structure (which leads to alterations in efflux protein conformation and activity).

Bioactive Metabolism or Chemical Transformation

Numerous molecules isolated from plant and animal sources have been shown to enhance the bioavailability of nutraceuticals or pharmaceuticals due to their ability to interfere with chemical transformations that normally occur within the GIT or after absorption. Some of these bioactivity enhancers act as antioxidants that retard the oxidation of nutraceuticals or pharmaceuticals, such as ω-3 fatty acids, carotenoids, or conjugated linoleic acid. For example, there are many natural and synthetic food-grade antioxidants that are effective at inhibiting oxidation reactions by mechanisms such as free radical scavenging, singlet oxygen quenchers, and chelating agents, e.g., BHT, BHA, carotenoids, tocopherols, flavonoids, and grade seed extract. Other bioactivity enhancers may inhibit the normal functioning of metabolic or digestive enzymes within the GIT or body. For example, piperine has been shown to retard the metabolism of certain drugs and nutraceuticals, such as ibuprofen, curcumin, resveratrol, EGCG, carotenoids, vitamins, and amino acids. These affects have been partly attributed to its ability to inhibit metabolizing enzymes such as glucose dehydrogenase, cytochrome P450, and others.

FIG. 3a shows a block diagram representation of a summary of the embodiments disclosed herein above.

Referring to FIG. 3a, in the embodiment shown therein, the three major factors disclosed herein above are applied in the design of an excipient food. The factors can all be applied jointly or separately.

TABLE 2
Examples of phytochemicals from natural sources that
may increase the bioavailability of co-ingested lipophilic
nutraceuticals and pharmaceuticals.

| Bioavailability | Nutraceuticals | |
|---|---|---|
| Enhancer | Enhanced | Mechanism |
| Piperine | Vitamins A, D, E, K | Metabolizing Enzyme Inhibition |
| | Carotenoids, | Modulation of Gut Motility |
| | Curcuminoids | |
| | Coenzyme Q10 | |
| | Hydrophobic drugs | |
| Gingerols | Vitamins A and E | Modulation of Gut Motility |
| | Carotenoids, | |
| | Curcumin | |
| Curcumin | Hydrophobic drugs | Metabolizing Enzyme Inhibition |
| | | Efflux Transporter Inhibition |
| Quercetin | Hydrophobic drugs | Efflux Transporter Inhibition |

Impact of Specific Food Ingredients

In this section, the potential influence of common food components that may be incorporated into excipient foods on the oral bioavailability of lipophilic bioactive agents is discussed. Those ingredients that appreciably increase the bioavailability of nutraceuticals can be referred to as “excipient food ingredients”. An excipient food may contain one or more of these ingredients so as to increase the bioavailability of one or more nutraceuticals.

Lipids

Studies by pharmaceutical researchers have shown that co-ingestion of lipophilic drugs with lipids improves theirs oral bioavailability by an amount that depends on the amount, type, and structure of the ingested lipids. Food and nutrition research has also shown that the bioavailability of lipophilic nutraceuticals can be increased by co-ingestion with lipids. In vitro studies have reported that the bioaccessibility (micelle solubilization) and absorption (cell culture uptake) of lipophilic bioactive agents from fruits and vegetables is greatly increased in the presence of lipids. The extent of the increase in bioaccessibility and absorption depends on the amount and composition of the lipids used. Bioaccessibility was higher for lipids containing long chain triglycerides (LCT) than those containing short or medium chain triglycerides (SCT or MCT), presumably because of differences in the solubilization capacity of the mixed micelles formed. Lipophilic bioactives encapsulated within indigestible oils (flavor oils) have been shown to have low bioaccessibility using in vitro studies, which was attributed to the fact that some of them remained in the undigested oil droplets and there were fewer mixed micelles available to solubilize them. In addition to their composition, the liberation of bioactives from emulsified lipids also depends on their particle size, physical state, and interfacial characteristics. Typically, the release rate is faster for smaller particles, for liquid oils rather than solid fats, and for interfaces where bile salts and lipases can easily absorb.

Co-ingested lipids may also alter the bioavailability of lipophilic drugs or nutraceuticals through other mechanisms. When lipophilic bioactives are ingested with LCT they are packed into lipoprotein particles (chylomicrons) in the intestinal epithelial cells and then transported by the lymphatic route (thereby avoiding first pass metabolism in the liver), but when they are ingested with SCT or MCT they tend to be transported via the portal vein (where they must pass through the liver before entering the systemic blood circulation). Bioactives packaged in different vehicles (e.g., chylomicron vs. non-chylomicron) in the epithelial cells may have different metabolic fates due to differences in their exposure to metabolizing enzymes present in different body tissues.

Carbohydrates

In general, food carbohydrates are classified as monosaccharides (n=1), oligosaccharides (n=2 to 20), or polysaccharides (n>20) depending on the number of monomers present. Carbohydrates may also be classified as digestible or indigestible depending on their susceptibility to enzymatic hydrolysis in the upper GIT. Starch is the most abundant digestible polysaccharide in foods, whereas there are many types of indigestible polysaccharides, such as cellulose, hemicellulose, pectin, alginate, carrageenan, xanthan gum, locust bean gum, and agar. Indigestible polysaccharides are part of a class of polymers known as dietary fibers, which vary according to their monomer type, distribution, and bonding, as well as their electrical charge, hydrophobicity, molecular weight, degree of branching, and conformation. Co-ingested carbohydrates may influence the bioavailability of lipophilic bioactive drugs and nutraceuticals through various mechanisms. As mentioned earlier, many polysaccharides are able to increase the viscosity or form a gel within the GIT, thereby altering mass transport processes, e.g., diffusion of enzymes to substrates in food matrices, or digestion products/bioactives to epithelial cells. Some dietary fibers may be able to form impermeable coatings around food matrix components that inhibit their digestion and therefore the release of bioactive agents. Electrically charged polysaccharides are capable of binding oppositely charged molecular species in the GIT that may influence food matrix digestion and bioactive release. For example, cationic dietary fibers (such as chitosan) can bind anionic bile salts, fatty acids, or phospholipids, whereas anionic dietary fibers (such as alginate) can bind cationic calcium ions. Cationic dietary fibers have also been shown to inhibit lipase activity, and therefore reduce the rate of lipid digestion. Some dietary fibers have been shown to alter cell membrane permeability through their effect on tight junction dimensions, e.g., chitosan. Dietary fibers may also change the nature of the microbial population within the colon, which can alter the metabolism, activity, and absorption of lipophilic bioactives in the large intestine.

Proteins

Food proteins exhibit a wide range of different molecular structures, physicochemical properties, and physiological effects. Co-ingested proteins can potentially alter the bioavailability of lipophilic bioactive agents through a number of mechanisms. Many food proteins and peptides have strong antioxidant activity and may therefore be able to inhibit the chemical degradation of nutraceuticals or drugs that are susceptible to oxidation within the GIT, such as ω-3 fatty acids or carotenoids. Some nutraceuticals may bind to proteins within the GIT, which alters the location of their absorption within the GIT, e.g., anthocyanins bound to proteins have been shown to travel further down the gastrointestinal tract. Protein digestion within the gastrointestinal tract may generate hormonal responses that regulate food intake and processing, thereby altering the way that a food or pharmaceutical matrix is broken down in the GIT and therefore the release of any trapped bioactive agents. Proteins and their digestion products may interact with various molecular species involved in the digestion of food matrices and the release and transport of bioactive agents, such as bioactives, mixed micelles, phospholipids, and enzymes. For example, a recent study suggests that lactoferrin may reduce the bioavailability of β-carotene, which was attributed to the fact that it was positively charged and bound to negatively charged digestive components, such as bile salts or free fatty acids. Some protein digestion products, for example those from casein and whey proteins, have been shown to alter (close) tight junction permeability, and may therefore alter the uptake of any nutraceuticals absorbed by this mechanism.

Surfactants

Surfactants are commonly used in the food and pharmaceutical industries to form and stabilize colloidal delivery systems, such as microemulsions, nanoemulsions, emulsions, and solid lipid nanoparticles. Surfactants vary in the nature of their polar head groups and non-polar tail groups, which alters their behavior within foods and the GIT. The head group may be non-ionic, cationic, anionic, or zwitterionic, while the tail group may vary in the number, length and unsaturation of the non-polar chains. Synthetic or natural surfactants may be present within an ingested food e.g., non-ionic surfactants (e.g., Tweens, Spans, and sucrose esters), ionic surfactants (e.g., DATEM and CITREM), phospholipids (e.g., egg, soy, or sunflower lecithin), or monoacylglycerols. Alternatively, they may be generated from ingested food components as a result of the digestion process, e.g., monoacylglycerols from triacylglycerols or lysolecithin from phospholipids. Surfactants can alter the bioavailability of lipophilic bioactives through a number of mechanisms: some surfactants bind to digestive enzymes (such as lipase or protease) and alter their activity; surfactants may be incorporated into mixed micelles thereby increasing their solubilization capacity; surfactants may inhibit lipase absorption to lipid surfaces through competitive absorption; surfactants may alter the permeability of enterocytes by interacting with transporters on cell membranes; surfactants may increase cell permeability by increasing the dimensions of the tight junctions.

Minerals

Certain types of mineral ions also impact the liberation and absorption of lipophilic bioactives. For example, calcium ions may impact the rate and extent of lipid hydrolysis, which influences the release of bioactives from the lipid phase and their subsequent solubilization in the mixed micelle phase. In the absence of calcium, the digestion of triacylglycerols in the small intestine is inhibited by accumulation of long-chain fatty acids (LCFA) at the oil-water interface, since this restricts the access of lipase to the lipid substrate. Calcium ions precipitate accumulated LCFAs through complexation, thereby removing them from the interface and allowing the lipase to access the lipid substrate. Calcium ions are therefore able to increase the rate and extent of lipid digestion through this mechanism. Conversely, the formation of calcium-LCFA precipitates may reduce the solubilization capacity of the mixed micelle phase, thereby reducing the bioavailability of LCFAs and lipophilic bioactives. Calcium has also been shown to play an important role in the activity of pancreatic lipase, acting as a co-factor required for activity. Multivalent mineral ions may promote the aggregation of oppositely charged lipid droplets, thereby altering the surface area of lipid exposed to digestive enzymes. Mineral ions may also promote gelation of oppositely charged biopolymers (e.g., calcium ions promote alginate gelation), which will also influence the accessibility of lipid phases to enzyme digestion. Some minerals have been shown to influence the absorption of bioactive agents by altering cell membrane permeability, e.g., zinc.

Chelating Agents

Metal ion chelators (such as EDTA) have been shown to inhibit efflux transporters in the GIT, and may therefore increase the bioavailability of bioactive molecules that are susceptible to removal from enterocytes by this mechanism. Metal ion chelators (such as EDTA and phosphates) may interfere with the various roles that calcium ions play in the digestion and release of lipids by complexing them.

Phytochemicals

A number of phytochemicals derived from edible plant materials have been shown to be able to promote the bioavailability of certain bioactive food agents. For example, some polyphenols affect absorption and efflux transporters in enterocyte membranes thus altering the accumulation of bioactive agents within the body e.g., quercetin, curcumin, piperine, and some catechins. Specific phytochemicals may also be able to inhibit chemical reactions (such as lipid oxidation) or biochemical reactions (such as digestion or metabolism) in the gastrointestinal tract. For example, it has been reported that piperine reduced the metabolism of curcumin in the GIT by inhibiting metabolizing enzymes, thereby increasing bioavailability.

Excipient Food Ingredients

Many of the food ingredients discussed in the previous sections have the ability to increase the oral bioavailability of co-ingested bioactive agents. These ingredients can therefore be used to construct excipient foods that are specifically designed to increase the overall oral bioavailability of one or more type of co-ingested bioactive agents. For example, an excipient food may contain lipids to increase the solubilization capacity of the intestinal fluids, a phytochemical to inhibit efflux mechanisms, and a surfactant to increase epithelium cell membrane permeability.

In one or more instances, the method of these teachings also includes using an in vitro GIT model to verify improvement of oral bioavailability when the predetermined pharmaceutical or nutraceutical is ingested with the food matrix.

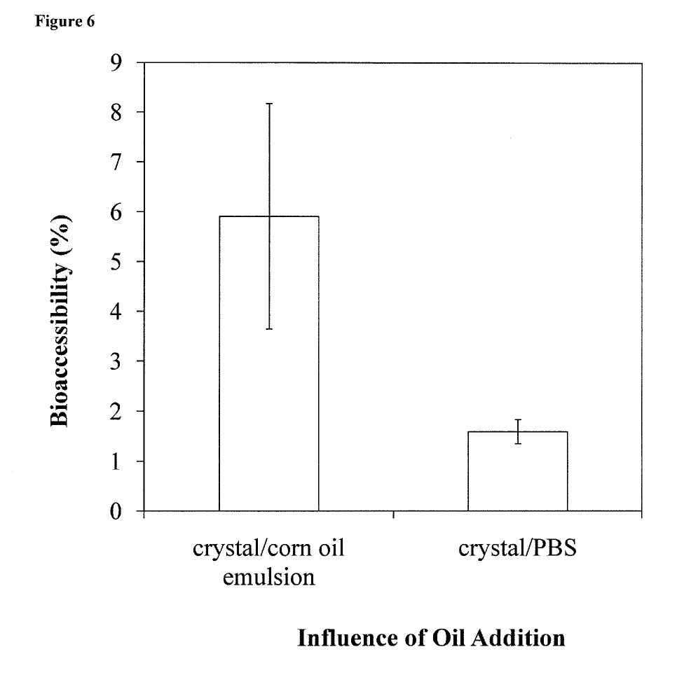

FIG. 6 shows influence of mixing β-carotene crystals with either an oil-in-water emulsion (containing 4% corn oil) or a buffer solution (PBS) containing no oil on the bioaccessibility of β-carotene using an in vitro digestion model. This data demonstrates that the corn oil emulsion acts as an excipient food that increases the bioaccessibility of the crystalline β-carotene, presumably by forming mixed micelles that solubilize this lipophilic nutraceutical.

In this exemplary embodiment, β-carotene crystals were initially mixed with either an oil-in-water emulsion (containing 4% corn oil) or a buffer solution (PBS) containing no oil. The resulting mixtures were then passed through an in vitro gastrointestinal model that simulated the mouth, stomach, and small intestine. The bioaccessibility of β-carotene was determined by centrifuging the digesta resulting from a GIT model, and then measuring the fraction of β-carotene in the mixed micelle (middle phase). The GIT model used is described in Salvia-Trujillo, L.; Qian, C.; Martin-Belloso, O.; McClements, D. J., “Influence of Particle Size on Lipid Digestion and β-carotene Bioaccessibility in Emulsions and Nanoemulsions”, Food Chemistry, 141:1472-80 (2013), which is incorporated by reference herein in its entirety and for all purposes. In another instance, the method of these teachings also include using an animal feeding study to verify improvement of oral bioavailability when the predetermined pharmaceutical or nutraceutical is ingested with the food matrix. An example of an animal feeding study is given in Cho, H. T., Salvia-Trujillo, L., Kim, J., Park, Y., Xiao, H., and McClements, D. J., “Droplet Size and Composition of Nutraceutical Nanoemulsions influences Bioavailability of Long Chain Fatty Acids and Coenzyme Q10”, Food Chemistry, 156:117-22 (2014), which is incorporated by reference herein in its entirety and for all purposes.

The following are examples of compounds that may benefit from the potential of food matrix effects to increase their bioavailability:
β-carotene (carotenoid)
curcumin (curcuminoid)
tangeretin (polymethoxyflavone)
polyunsaturated fatty acids
nobiletin (polymethoxyflavone)
Vitamin E
Resveratrol (stilbene)
Pterostilbene (stilbene)

Nutraceuticals with poor oral bioavailability (such as, but not limited to, iron, calcium, other carotenoids, other oil-soluble vitamins (A, D, and K), peptides, and proteins) may also benefit from the potential of food matrix effects to increase their bioavailability.

Potential Benefits of Excipient Foods

There are several potential benefits of developing excipient foods to increase the oral bioavailability of nutraceuticals and drugs. The long-term consumption of low levels of nutraceuticals may improve human performance, enhance wellbeing, or inhibit the onset of chronic diseases, such as heart disease, diabetes, hypertension, and cancer. This would increase the quality of life of the general population and reduce the costs of health care associated with treatment of these chronic diseases. At present, the bioavailability of the nutraceuticals in many natural sources, such as fruits and vegetables, is relatively low, and therefore their potential benefits on long-term human health are not being fully realized. In addition, it is well established that the oral bioavailability of many lipophilic drugs is relatively low and variable, which reduces their efficacy and can lead to undesirable side effects. The development of specially designed excipient foods that enhance bioavailability and bioactivity may be able to overcome these problems.

The development of successful excipient foods faces a number of technical, legal, and commercial challenges. In particular, there are important differences in the ability to prove the impact of excipient foods on the bioactivity of drugs and nutraceuticals. Drugs can be administered in well-defined doses at specified times thereby enabling pharmaceutical researchers to carry out studies to establish their efficacy against specific disease symptoms or biomarkers. Thus the impact of excipient foods on drug bioactivity can be established using well-controlled experiments that involve taking the drug in the absence or presence of the excipient food. In contrast, nutraceuticals are typically consumed at relatively low levels as part of a complex diet over extended periods. Hence, it is often difficult to establish a strong correlation between the type and amount of nutraceutical consumed and a particular disease. This would make it challenging to prove the efficacy of excipient foods at improving human health and wellness since long-term studies would be needed with well-controlled diets. Consequently, it would be difficult for food manufacturers to provide the scientific evidence required by regulators to make specific health claims about an excipient food product in their advertising or labeling. In the absence of this kind of competitive advantage food companies may be reluctant to spend research funds on developing and testing the efficacy of excipient foods. Nevertheless, one might be able to make the simpler claim that excipient foods increase the bioavailability of specific food components, such as carotenoids or oil-soluble vitamins. Another unique challenge that the food industry faces is in controlling the dose and timing that a nutraceutical containing food and an excipient food are consumed. Pharmaceuticals are usually taken in well-defined doses at specific times, whereas nutraceutical agents may be present in various types of foods that are consumed in different amounts by different individuals as part of a complex diet that contains other components that could affect bioavailability. The time that an excipient food is consumed relative to a nutraceutical-rich food may also be important for the efficacy in enhancing bioavailability, e.g., before, during, or after consumption.

Another potential challenge is that an individual may consume a number of different kinds of foods containing nutraceuticals, or a patient may need to take more than one kind of drug per day. It may be necessary to design different kinds of food matrices in excipient foods for different kinds of nutraceutical-rich foods or drugs. In addition, different individuals or patients have different food preferences and so a range of different kinds of excipient product types may be required, e.g., fruit drinks, yogurts, candies, deserts, spreads with different flavors.

Another potential issue with the development of excipient foods is their potential adverse side effects on human health. For example, the metabolizing enzymes and efflux transports in epithelial cells usually protect the human body from the effects of any harmful substances that have been ingested. If bioactive agents are incorporated into foods that appreciably alter these mechanisms, then they might increase the uptake of harmful substances that could have adverse effects on health and wellness. For example, some excipient food ingredients could increase the bioavailability of toxic substances found in foods. In addition, certain bioactive components may be beneficial to human health in relatively low doses, but have adverse effects at relatively high levels. In this case, the ability of an excipient food to greatly enhance the bioavailability of a bioactive component could be detrimental.

Finally, if excipient foods are going to be marketed to consumers, it will be important to educate them about their potential risk and benefits, and to provide advice about which excipient food should be consumed with which nutraceutical-rich food. For example, a dessert cream may be marketed as an excipient food to be consumed with berries, whereas a salad dressing may be marketed as an excipient food to be consumed with salads and vegetables (Table 1).

For the purposes of describing and defining the present teachings, it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

One or more embodiments relate to a method for improving oral bioavailability of pharmaceuticals or nutraceuticals, the method comprising:

designing a food matrix, the food matrix not having bioactivity above its normal nutritional function, that increases bioavailability of a predetermined pharmaceutical or nutraceutical by at least one of facilitating the release and solubilization of bioactive agents in the predetermined pharmaceutical or nutraceutical, altering the absorption of lipophilic bioactive agents in the predetermined pharmaceutical or nutraceutical, or interfering with chemical transformations that occur within gastrointestinal tract (GIT) or after absorption; the food matrix being co-ingested with the predetermined pharmaceutical or nutraceutical or being ingested at a specified time soon before or soon after the pharmaceutical or nutraceutical.

In some instances, the facilitating the release and solubilization of bioactive agents in the predetermined pharmaceutical or nutraceutical comprises at least one of enhancing breakdown of a matrix surrounding a bioactive agent, enhancing solubilization with a mixed micelle phase, altering mass transport processes within the GIT, or altering the motility of the GIT.

In other instances, the altering the absorption of lipophilic bioactive agents in the predetermined pharmaceutical or nutraceutical comprises at least one of increasing transport across a layer of epithelial cells surrounding the GIT or inhibiting the efflux mechanisms in membranes of intestinal epithelial cells.

In further instances, the method uses an in vitro GIT model or animal feeding study to verify improvement of oral bioavailability of the predetermined pharmaceutical or nutraceutical.

One or more embodiments relate to a composition that is a food matrix, the food matrix not having bioactivity above its normal nutritional function, the food matrix being designed to increases bioavailability of a predetermined pharmaceutical or nutraceutical.

In some instances, the bioavailability is increased by at least one of facilitating the release and solubilization of bioactive agents in the predetermined pharmaceutical or nutraceutical, altering the absorption of lipophilic bioactive agents in the predetermined pharmaceutical or nutraceutical, or interfering with chemical transformations that occur within gastrointestinal tract (GIT) or after absorption.

In other instances, the release and solubilization of bioactive agents in the predetermined pharmaceutical or nutraceutical is facilitated by at least one of enhancing breakdown of a matrix surrounding a bioactive agent, enhancing solubilization with a mixed micelle phase, altering mass transport processes within the GIT, or altering the motility of the GIT.

In still further instances, the absorption of lipophilic bioactive agents in the predetermined pharmaceutical or nutraceutical is altered by at least one of increasing transport across a layer of epithelial cells surrounding the GIT or inhibiting the efflux mechanisms in membranes of intestinal epithelial cells.

In yet other instances, the nutraceutical is curcumin and the food matrix is one of an emulsion, oil, or a buffer solution.

In yet other instances, the nutraceutical is β-carotene or α-carotene and the food matrix is one of an emulsion, oil, or a buffer solution.

In yet other instances, the nutraceutical is coenzyme Q10 and the food matrix is one of an emulsion, oil, or a buffer solution.

In still other instances, the nutraceutical is long chain fatty acids and the food matrix is one of an emulsion, oil, or a buffer solution.

One or more embodiments relate to a composition comprising an excipient emulsion comprising an aqueous buffer solution, a surfactant, an oil and one or more nutraceuticals or pharmaceuticals.

In some instances, the surfactant can be a GRAS compound. One example is Tween 80.

In some instances, the oil is a digestible oil, a indigestible oil or a mixture thereof. The digestible oil can be a vegetable oil, such as, but not limited to, corn oil or olive oil.

In some instances, the indigestible oil is mineral oil or a flavor oil. The flavor oil can be, but is not limited to, lemon oil or orange oil.

In some instances, the one or more nutraceutical or pharmaceutical has poor oral availability. The one or more nutraceutical can be iron or calcium. Alternatively, the one or more nutraceutical can be carotenoids, curcuminoids, polymethoxyflavones, stilbenes, vitamins, coenzymes, phytosterols/stanols, flavonoids, CLA, ω-3 Oils, polyunsaturated fatty acids, long chain fatty acids or mixtures thereof. Carotenoids include α-carotene, β-carotene or mixtures thereof. Curcuminoids include curcumin. Polymethoxyflavones include tangeretin, nobiletin or mixtures thereof. Stilbenes include resveratrol, pterostilbene or mixtures thereof. Oil soluble vitamins include vitamin A, vitamin D, vitamin E, vitamin K or mixtures thereof. Coenzymes include coenzyme Q10.

In some instances the pharmaceutical is a hydrophobic drug.

Another embodiment relates to a method of making an excipient emulsion comprising the steps of:

adding a surfactant to a buffer to form an aqueous phase;

homogenizing a digestible oil, an indigestible oil, or a mixture of a digestible oil and an indigestible oil, with the aqueous phase to form a coarse emulsion;

passing the coarse emulsion through a microfluidizer; and

adding a nutraceutical or a pharmaceutical having poor oral availability.

Although these teachings has been described with respect to various embodiments, it should be realized these teachings is also capable of a wide variety of further and other embodiments within the spirit and scope of these teachings.

### EXAMPLES

### Example 1

### Influence of Carrier Oil Composition (Digestible or Indigestible Oil) on β-Carotene Bioavailability

β-carotene has been reported to be the most important pro-vitamin A carotenoid found in foods and beverages. It is naturally present in many orange, red, yellow, and green colored fruits and vegetables. The results of epidemiological studies suggest that dietary intake of β-carotene may reduce the risk of certain chronic diseases, such as eye health, cancer and cardiovascular disease. There has therefore been considerable interest in understanding the major factors that influence the oral absorption of β-carotene in humans. It has been reported that the bioavailability of β-carotene is relatively low and highly variable. A number of physicochemical and physiological mechanisms have been proposed to account for this phenomenon: β-carotene is trapped within the organic matrix of many natural foods, which inhibits its release within the gastrointestinal tract (GIT); β-carotene is highly insoluble in water and only slightly soluble in oil at ambient temperatures, which limits its incorporation into many foods and may cause its precipitation within the GIT; β-carotene must be released from the food matrix and incorporated into mixed micelles before it can be absorbed; β-carotene is highly susceptible to oxidation due to its conjugated double bond structure, which means that it may degrade during storage. The low and variable oral bioavailability of β-carotene poses a challenge to the successful development of functional foods designed to improve human health and wellness.

The influence of carrier oil composition on the potential biological fate of β-carotene enriched nanoemulsions was investigated under simulated GIT conditions. In particular, how the ratio of digestible to indigestible oil within the oil phase influenced the bioaccessibility of β-carotene was examined. Here, the bioaccessibility is defined as the fraction of an ingested component that is successfully transferred from the food matrix into the mixed micelles formed by bile salts, phospholipids, and lipid digestion products i.e., free fatty acids and monoacylglycerols.

β-carotene enriched nanoemulsions were formed using “label friendly” surfactants (sucrose monoester and lysolecithin) suitable for stabilizing oil-in-water nanoemulsions. The oil phase of the nanoemulsions consisted of a mixture of corn oil and lemon oil. Corn oil is mainly comprised of triacylglycerol (TAG) molecules with long chain fatty acids (C₁₆ and C₁₈) attached to the glycerol backbone. In the presence of lipase the triacylglycerol molecules in corn oil are converted into monoacylglycerols (MAG) and free fatty acids (FFA) in the small intestine. These lipid digestion products can form mixed micelles with bile salts and phospholipids secreted in the intestinal fluids. These mixed micelles play an important role in the solubilization and transport of lipophilic bioactive components to the epithelium cells. Lemon oil mainly consists of monoterpenes, sesquiterpenes and their oxygenates, which will not be hydrolyzed by lipase within the GIT and will therefore not form mixed micelles.

Materials: Corn oil was used as an example of a long chain triglyceride (LCT) and was purchased from a local supermarket. Lemon oil (5-fold) was used as an example of an indigestible oil and was donated by Citrus & Allied Essences (Lake Success, N.Y.). Sucrose monopalmitate (SMP) and lysolecithin were provided by Compass Foods Company (Singapore). Beta-carotene (Type I, C9750) was purchased from the Sigma Chemical Company (St. Louis, Mo.). Lipase (from porcine pancreas pancreatin) and bile extract (porcine) were also obtained from Sigma. This lipase was reported to be a crude extract that contained a variety of other digestive components. All other chemicals used were of analytical grade. Double distilled water was used to prepare all solutions and emulsions.

β-Carotene-Enriched Nanoemulsion Preparation

Oil Phase Preparation: Initially, a series of carrier oils was prepared by mixing different ratios of corn oil and lemon oil together. Oil phases were then prepared by dispersing 5 g kg⁻¹ of powdered β-carotene into the carrier oils followed by heating at 50° C. for 5 min and stirring at ambient temperature for about 1 hour to ensure full dissolution of the carotenoid.

Aqueous Phase Preparation: Surfactant solutions were formed by mixing 0.4 g powdered sucrose monopalmitate (SMP) with 95.5 g water, followed by heating at 45° C. for 15 min to ensure SMP dissolution, then cooling to ambient temperature. Then 0.1 g lysolecithin was added into the SMP solution and the system was stirred at ambient temperature for about half an hour to ensure full dissolution.

Emulsion Preparation: Coarse emulsions were formed by mixing oil phase (40 g kg⁻¹) and aqueous phase (960 g kg⁻¹) in a container, and then blending them for 2 minutes at ambient temperature using a high-speed mixer (Tissue-Tearor, Biospec Products, Bartlesville, Okla.). Coarse emulsions were then passed through a high pressure homogenizer for three passes at 9,000 psi (Model M-110L Microfludizer Processor, Microfluidics, Newton, Mass.) to form a series of 40 g kg⁻¹ oil-in-water nanoemulsions containing the same β-carotene content (0.2 g kg⁻¹) but different oil phase compositions (digestible to indigestible oil ratio).

Particle Characterization

The mean particle diameters (d₄₃) and particle size distribution (PSD) of the nanoemulsions were measured after each digestion step using a static light scattering instrument (Mastersizer 2000 Malvern Instruments). A few drops of sample were dispersed in approximately 125 ml distilled water in the sample chamber with agitation until approximately 11-13% obscuration was obtained. The size of the particles in the micelle phase was measured using dynamic light scattering (ZetaSizer Nano, Malvern Instruments). The micelle phase was collected by centrifugation of the digesta after the simulated GIT model, followed by filtration through a 450 nm filter.

The electrical charge (ζ-potential) of the emulsion after each step digestion was measured using a micro-electrophoresis instrument (ZetaSizer Nano, Malvern Instruments). The ζ-potential was calculated from measurements of the electrophoretic mobility of droplets in an applied oscillating electric field using laser Doppler velocimetry. Samples were diluting using distilled water (initial, mouth, and small intestine samples) or pH 3 buffer solution (stomach) and then placed into disposable capillary cells (DTS 1060, Malvern Instruments).

Microstructural Analysis

A Nikon optical microscope (C1 Digital Eclipse, Tokyo, Japan) with a 40× objective lens (NA 0.75) was used to monitor the microstructure of the samples after each step of digestion. An aliquot of sample was sandwiched between a slide and a cover slip. The slide was then loaded onto the microscope stage and images were recorded.

Simulated Gastrointestinal Tract Model

Each β-carotene-enriched nanoemulsion was passed through a three-step simulated GIT model that consisted of a mouth, gastric, and small intestine phase. The particle size, charge, and microstructure of the samples were measured after incubation in each stage.

Mouth phase: Simulated saliva fluid (SSF), containing 30 g kg⁻¹ mucin and various inorganic salts was prepared as described in Mao, Y. and McClements, D. J., “Influence of electrostatic heteroaggregation of lipid droplets on their stability and digestibility under simulated gastrointestinal conditions”, Food & Function, (2012) and Sarkar, A., Goh, K. K. T. and Singh, H., “Colloidal stability and interactions of milk-protein-stabilized emulsions in an artificial saliva”, Food Hydrocolloids, 23:1270-78 (2009), both of which are incorporated herein in their entirety. 10 ml of nanoemulsion was placed in a conical flask (125 ml) and mixed with 10 ml of SSF so that the final mixture contained 20 g kg⁻¹ oil. The mixture was then adjusted to pH 6.8 and shaken continuously at 100 rpm in a temperature controlled incubator (37° C.) for 10 min to mimic oral conditions (Innova Incubator Shaker, 132 Model 4080, New Brunswick Scientific, New Jersey, USA).

Gastric phase: Simulated gastric fluid (SGF) was prepared by dissolving 2 g NaCl, 7 mL HCl, and 3.2 g pepsin (from porcine gastric mucosa) in a flask and then diluting with double distilled water to a volume of 1 L, and finally adjusting the pH to 1.2 using 1.0 M HCl. The “bolus” sample from the mouth phase was then mixed with SGF at a 1:1 mass ratio so that the final mixture contained 10 g kg⁻¹ oil. The mixture was then adjusted to pH 2.5 using 1M NaOH and incubated with shaking continuously at 100 rpm in a temperature controlled incubator (37° C.) for 2 h to mimic stomach conditions. It should be noted that we did not include a gastric lipase in this study, which is known to promote a limited amount of lipid digestion within the stomach of humans.

Small Intestine phase: Digesta samples (30 ml) obtained from the simulated gastric phase were placed in a beaker incubated in a water bath (37° C.) for 10 min and then adjusted to pH 7 with NaOH solution (0.05 or 1 M). The simulated small intestinal fluid (SSIF) contained 2.5 ml pancreatic lipase (1.6 mg ml⁻¹), 4 ml bile extract solution (5 mg ml⁻¹) and 1 ml CaCl₂ solution (750 mM). The 4 ml bile extract solution was first added into the 30 ml digesta with stirring and the resulting system was adjusted to pH 7.0. Then 1.0 ml of CaCl₂ solution was added and the system was adjusted to pH 7.0. Finally, 2.5 ml of freshly prepared pancreatic suspension was added to the mixture. At this point, an automatic titration experiment was started. A pH-stat titration unit (Metrohm, USA Inc.) was used to monitor and control the pH (at pH 7.0) of the digestion solution. The volume of NaOH solution (0.25 M) added to keep the pH constant was assumed to be related to the amount of free fatty acids generated by lipolysis of the initial triacylglycerol. Digestion experiments were performed for 2 h. The amount of free fatty acids released was calculated from the titration curves as described in Li, Y. and McClements, D. J., New Mathematical Model for Interpreting pH-Stat Digestion Profiles: Impact of Lipid Droplet Characteristics on in Vitro Digestibility”, Journal of Agricultural and Food Chemistry, 58:8085-92 (2010), which is incorporated herein in its entirety. The release of free fatty acids from the digestible oil (corn oil) was calculated by subtracting the FFA concentration determined at each time point for the pure lemon oil nanoemulsions from the FFA concentration determined for the nanoemulsions containing corn oil. In other words, the lemon oil nanoemulsion was used as a blank to account for any pH changes caused by other substances present within the reaction vessel during digestion.

Bioaccessibility Determination

After in vitro digestion, samples were collected and centrifuged (4000 rpm, Thermo Scientific, CL10 centrifuge) at 25° C. for 40 min. Centrifuged samples separated into an optically opaque orange sediment phase at the bottom, a relatively clear aqueous phase in the middle, and sometimes an oily or creamed phase at top. The middle phase was assumed to consist of mixed micelles that solubilized the β-carotene. The β-carotene was extracted using isopropanol-isooctane solution (1:1 mass ratio) as an organic solvent. 0.2 ml of micelle phase or total digesta from the small intestinal was mixed with 4.8 ml isopropanol-isooctane solution and centrifuged at 5000 rpm at 25° C. for 5 min. The β-carotene concentration was analyzed using a UV-visible spectrophotometer (Ultrospec 3000 pro, GE Health Sciences, USA) at 450 nm. The isopropanol-isooctane solution was used as a reference. The bioaccessibility was calculated using the following equation:

$$Bioaccessibility = 100 ( \frac{{C}_{Micelle}}{{C}_{Digesta}} ) \qquad ( 1 )$$

where, C_{micelle} and C_{Digesta} are the concentrations of β-carotene in the micelle fraction and in the overall sample (total digesta) after the pH-stat experiment, respectively.

Statistical Analysis

All measurements were performed at least twice using freshly prepared samples and were reported as calculated means and standard deviations.

Results and Discussion

Physical Stability of Nanoemulsions within a Simulated GIT Model

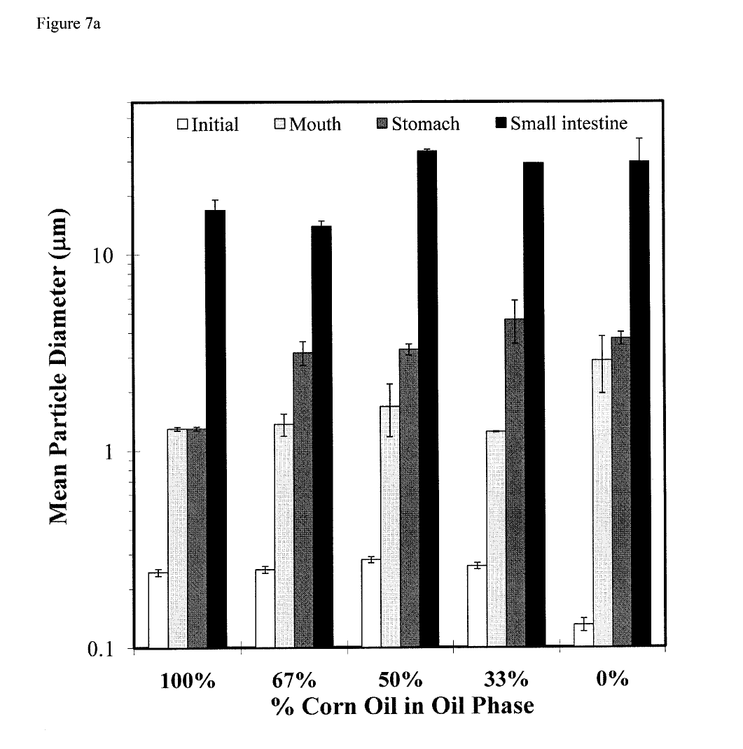

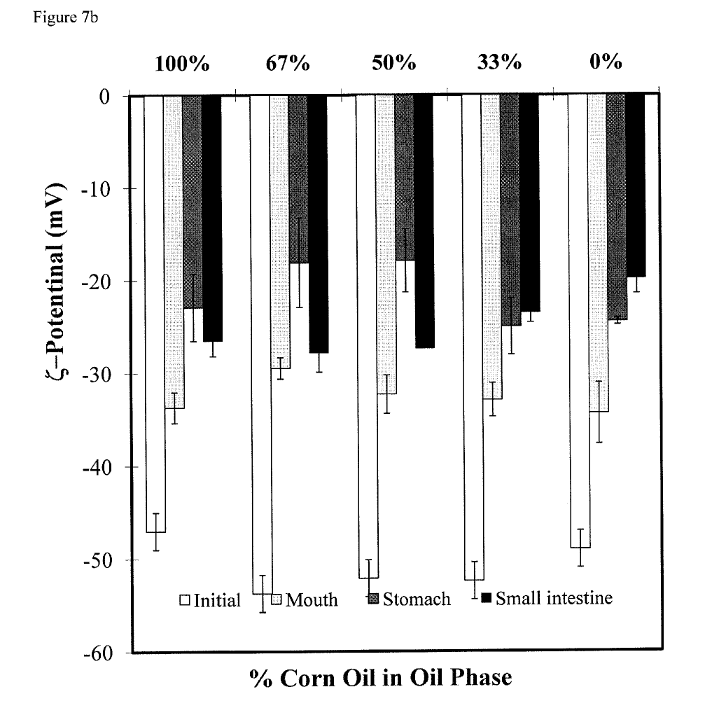

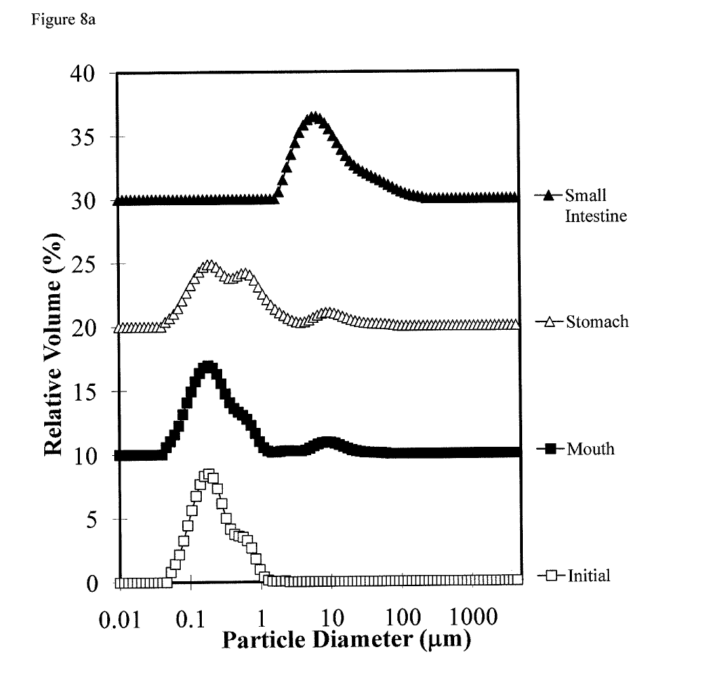

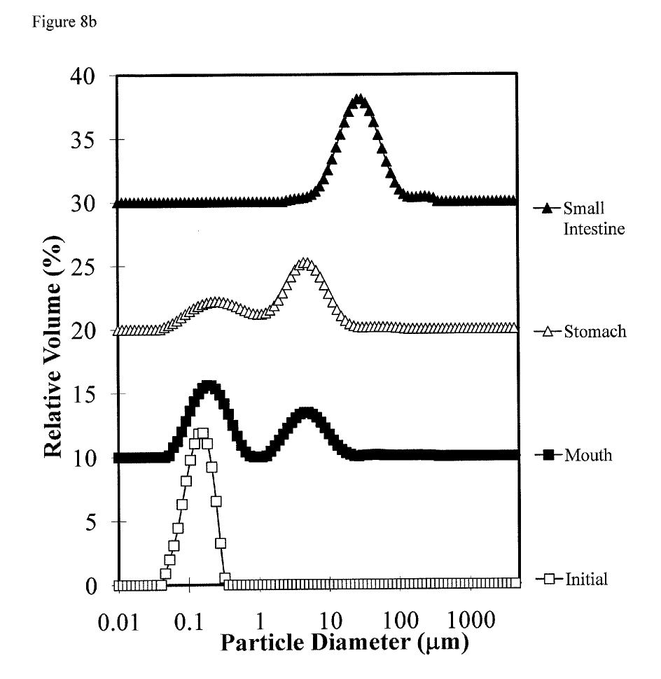

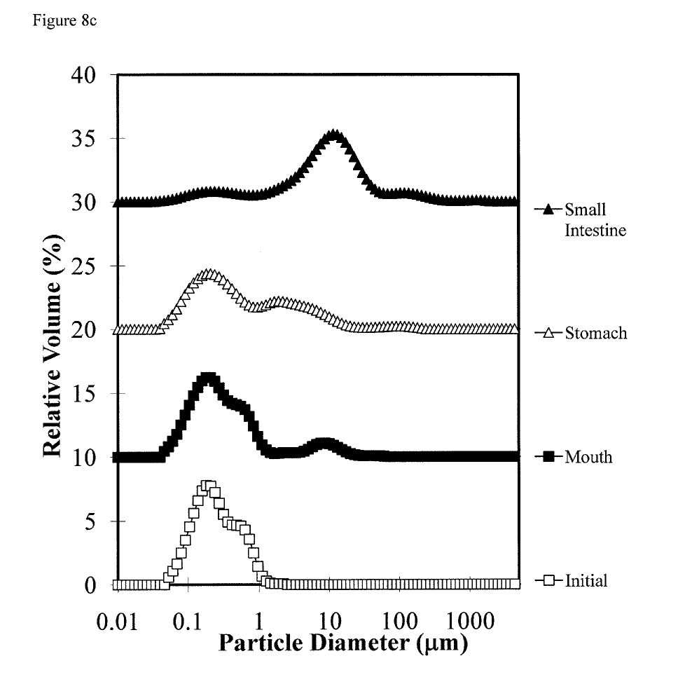

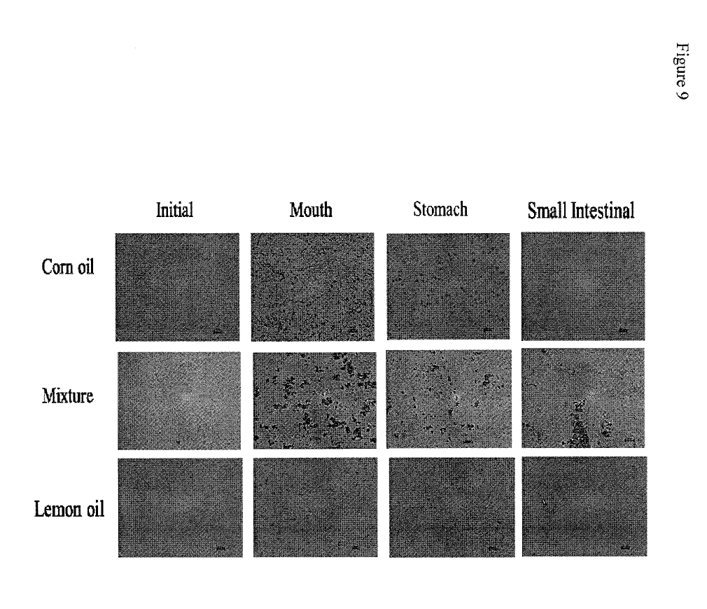

The influence of carrier oil composition on the potential biological fate of β-carotene-enriched nanoemulsions was investigated using a simulated gastrointestinal tract (GIT) model. The initial nanoemulsions contained 40 g kg⁻¹ total oil (5 g kg⁻¹ β-carotene in carrier oil) and 5 g kg⁻¹ total surfactant (4 g kg⁻¹ SMP+1 g kg⁻¹ lecithin). Nanoemulsions containing different ratios of digestible and indigestible oils were formed by mixing varying ratios of corn oil and lemon oil together prior to homogenization. The mean particle diameter (FIG. 7a), particle size distribution (FIGS. 8a-8c), particle charge (FIG. 7b), and microstructure (FIG. 9) of the samples were measured after each stage of the GIT model.

Initial nanoemulsions: All the initial nanoemulsions had relatively small mean particle diameters (d₄₃=0.13 to 0.24 μm) (FIG. 7a), monomodal particle size distributions (FIGS. 8a-8c), and uniform microstructures (FIG. 9), which suggested that they were stable to droplet aggregation under the initial solution conditions (pH 7). The mean particle diameter was appreciably lower for the nanoemulsions prepared using pure lemon oil, which can be attributed to more efficient droplet disruption during homogenization for lemon oil. The initial nanoemulsions all had relatively high negative charges (−47 to −54 mV) regardless of carrier oil composition (FIG. 7b), which can be attributed to the anionic nature of the lecithin and sucrose monopalmitate surfactants adsorbed to the droplet surfaces. In principle, SMP is a non-ionic surfactant, but previous studies have shown that SMP-coated droplets do have an appreciable negative charge at neutral pH (presumably due to impurities such as palmitic acid).

Mouth phase: For all nanoemulsions after incubation in artificial saliva (pH 7), the mean particle diameter increased appreciably (d₄₃>1 μm) (FIG. 7a), a population of larger droplets was observed in the particle size distributions (FIGS. 8a-8c), and evidence of extensive flocculation was observed in the optical microscopy images (FIG. 9). These results suggest that all the nanoemulsions were susceptible to droplet aggregation within the simulated mouth phase regardless of oil phase composition. The optical microscopy images suggest that the increase in droplet size was most likely caused by flocculation, i.e., clustering together of the individual oil droplets. There was also an appreciable decrease in the magnitude of the negative charge on the particles in all the nanoemulsions after incubation in the simulated mouth phase: from around −50 to −30 mV (FIG. 7b). It has been suggested that changes in droplet aggregation and charge within the mouth phase can be attributed to the presence of salts and mucin within the saliva. Salts may reduce the ζ-potential of droplets due to electrostatic screening or ion binding effects, whereas mucin may change the droplet charge by adsorbing to the droplet surfaces. As a result, there may be a decrease in the electrostatic repulsion between the droplets leading to flocculation. In addition, mucin may have promoted droplet flocculation through a bridging or depletion mechanism. It has also been reported that SMP-coated oil droplets undergo extensive aggregation when mixed with salts.

Gastric phase: There were major changes in the particle size and microstructure of all the emulsions after they were incubated in simulated gastric fluid (SGF) for two hours. There was an increase in the mean particle diameter (FIG. 7a), larger particles present in the particle size distributions (FIGS. 8a-8c), and evidence of aggregate formation in the optical microscopy images (FIG. 9). These results suggest that incubation of the nanoemulsions within the highly acidic environment of the stomach did not breakdown the aggregates formed within the mouth, and may even have promoted further droplet aggregation. There was an appreciable decrease in the magnitude of the negative charge on the particles in all the emulsions when then went from the mouth to the stomach phase (FIG. 7b). This effect can mainly be attributed to the decrease in the pH of the aqueous phase surrounding the oil droplets, since previous studies have reported that the negative charge on emulsion droplets stabilized by lecithin and SMP decreases with decreasing pH. In addition, the presence of salts in the simulated gastric fluids would have reduced the ζ-potential on the droplets due to electrostatic screening and ion binding effects. A reduction in the magnitude of the droplet charge would potentially decrease the strength of the electrostatic repulsion between the oil droplets, thereby promoting aggregation. There may also have been some hydrolysis of the sucrose monoester within the highly acidic environment of the gastric phase, which would have led to emulsion instability.

Small Intestine phase: Lipid digestion primarily occurs within the small intestine due to the presence of pancreatic lipases capable of hydrolyzing the triacylglycerol molecules. Bioactive components may then be solubilized by the intestinal fluids due to the presence of endogenous (bile salts and phospholipids) and exogenous (monoacylglycerols and free fatty acids) surface active molecules that form mixed micelles and other association colloids. Therefore the influence of small intestine conditions on the physical characteristics of the particles was examined in the different samples.

After incubation in simulated small intestine fluids, all the samples contained large particles, as seen from the particle size (FIGS. 7a and 8a-8c) and microscopy (FIG. 9) measurements. The presence of large particles can be explained by several mechanisms. Triacylglycerol molecules present within the oil droplets may be converted into long chain free fatty acids and monoacylglycerols due to hydrolysis by pancreatic lipase. Therefore there could be a decrease in the size and number of oil droplets remaining in the system as the digestion products move into the surrounding aqueous phase. Nevertheless, some droplet coalescence may occur during the lipid digestion process due to changes in interfacial or oil phase composition, leading to an overall increase in oil droplet size. In addition, the digestion products that leave the oil phase may assemble into other types of colloidal structure, such as mixed micelles (small), vesicles (large) and calcium salts (large). Finally, it is also possible that there was some hydrolysis of the ester bond in the sucrose monoesters in the presence of lipase, which reduced the surface-activity of the SMP surfactant, and therefore led to droplet coalescence.

The electrical charge on the particles in all the systems was negative (−20 to −29 mV) after incubation in the small intestine phase (FIG. 7b). This negative charge may be attributed to the presence of a number of different anionic surface-active species in the nanoemulsions after digestion, including bile salts, phospholipids, free fatty acids, and lysolecithin. The negative charge tended to increase as the amount of corn oil within the initial lipid phase increased, which may have been due to the greater amount of anionic free fatty acids produced by hydrolysis.

Influence of Carrier Oil Composition on Lipid Digestion

The influence of carrier oil composition (digestible-to-indigestible oil) on the rate and extent of lipid digestion in the simulated small intestine was monitored using a pH-stat method. The pH-stat method measures the amount of free fatty acids (FFAs) released from triacylglycerols due to the action of lipase. The pure lemon oil nanoemulsion was used as a control since it contains no digestible triacylglycerols, being mainly composed of monoterpenes, sesquiterpenes and their oxygenates.

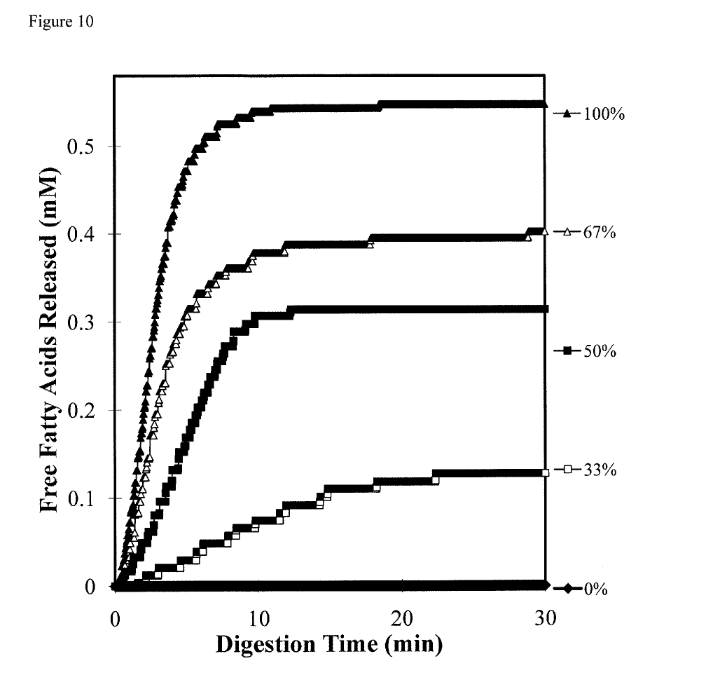

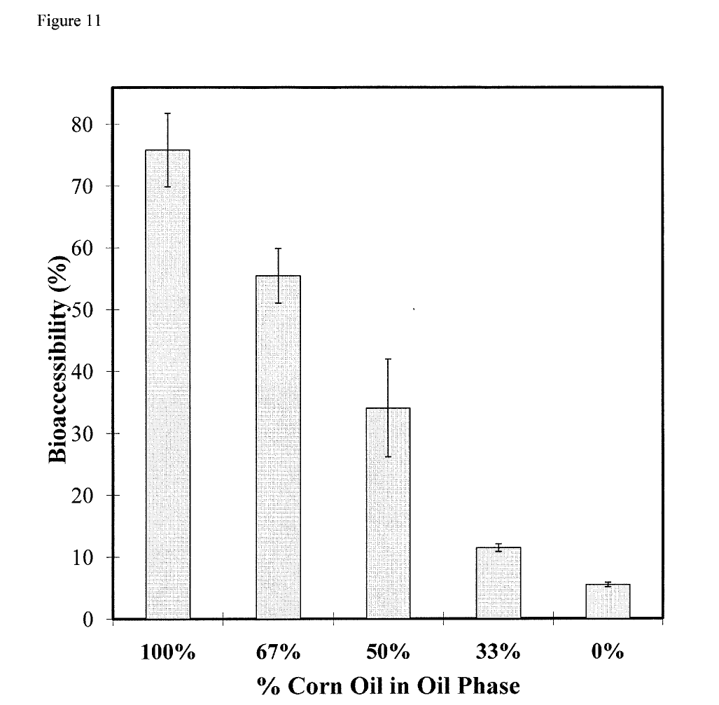

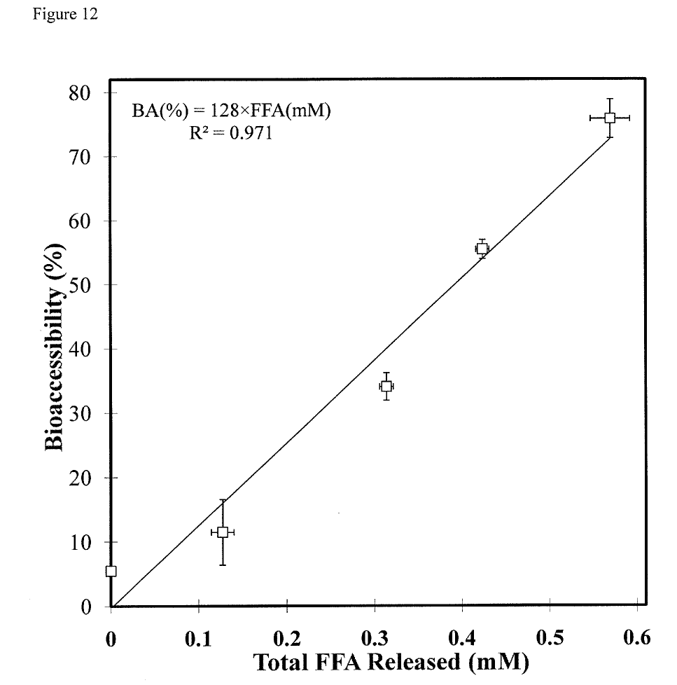

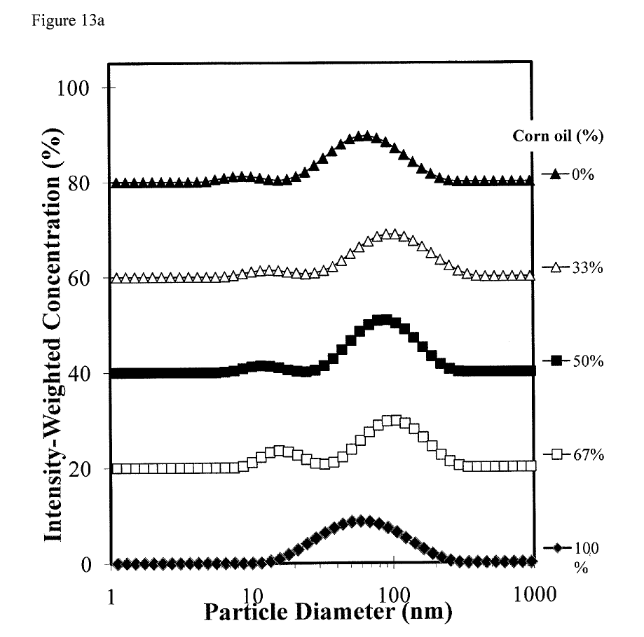

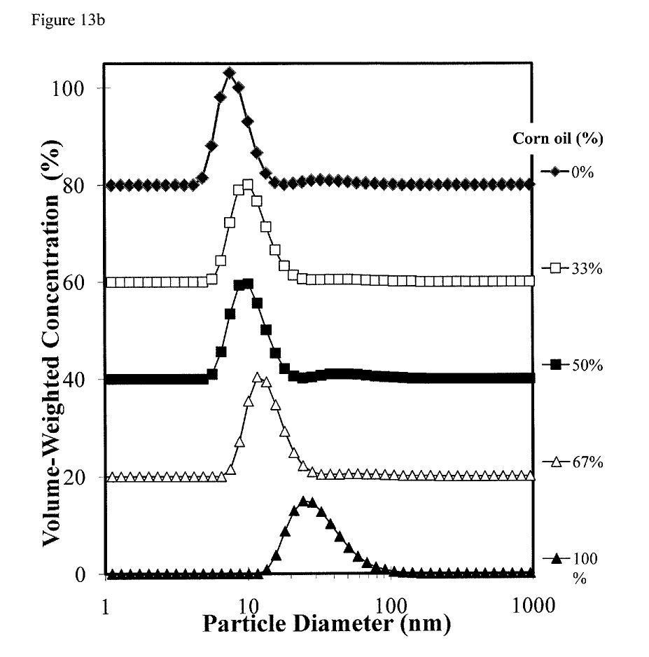

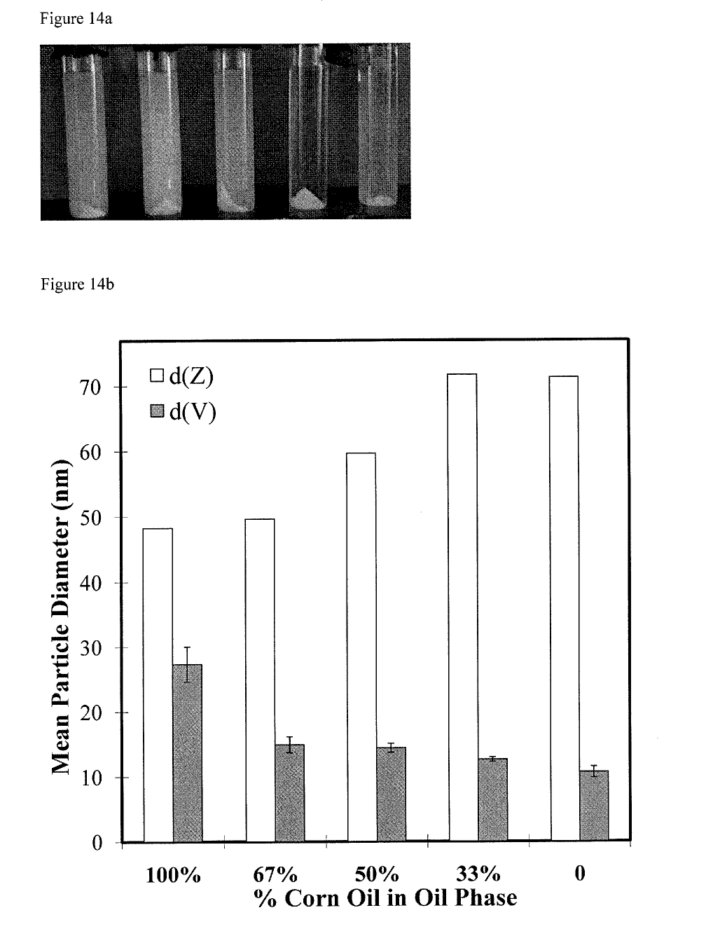

In general, the free fatty acid versus time profiles showed similar trends for all nanoemulsions containing digestible oil i.e., corn oil (FIG. 10). There was an appreciable increase in FFAs during the first 20 minutes, followed by a more gradual increase at longer digestion times, until a plateau value was attained. The total amount of FFAs released (in mmols) increased as the total amount of corn oil in the lipid phase increased. Nevertheless, calculations of the amount of FFAs released compared to the total amount of FFAs that could be released assuming 2 FFAs per TAG molecule (Φ) indicated that not all of the triacylglycerols were digested: Φ=76±3%, 84±2%, 84±2%, and 51±5%, for nanoemulsions containing 100, 67, 50 and 33% corn oil in the lipid phase, respectively. These relatively low numbers, may have been because some digestion of the TAGs had already occurred within the stomach prior to entering the small intestine, or it may have been because the presence of long-chain fatty acids at the droplet surfaces inhibited surface hydrolysis. The observation that only about half of the triacylglycerols were digested in the nanoemulsions containing 67% lemon oil and 33% corn oil in the lipid phase suggests that lemon oil inhibited TAG digestion. This may have occurred because the triacylglycerol molecules were completely surrounded by lemon oil molecules, which prevented the lipase from reaching the ester bonds in the TAGs.

Influence of Carrier Oil on In Vitro β-Carotene Bioaccessibility

Finally, the influence of carrier oil composition on β-carotene bioaccessibility was examined after the nanoemulsions had been passed through the simulated GIT model. A series of nanoemulsions containing 5 g kg⁻¹ β-carotene in the carrier oil were prepared and then passed through the mouth, stomach, and small intestine phases. The concentration of carotenoid solubilized within the micelle phase was then measured, and the bioaccessibility was calculated as the fraction of β-carotene present within the micelle phase compared to the total digesta.

The initial carrier oil composition had an appreciable influence on the bioaccessibility of the β-carotene in the nanoemulsions. The bioaccessibility of β-carotene decreased as the percentage of digestible triacylglycerols in the oil phase decreased, falling from around 76% to 5% as the corn oil content decreased from 100% to 0% (FIG. 5). Interestingly, there appeared to be a linear relationship between β-carotene bioaccessibility and the amount of free fatty acids released from the nanoemulsions (FIG. 6). These results highlight the critical role of mixed micelles in solubilizing carotenoids. The lowest bioaccessibility was measured in the nanoemulsions prepared using pure lemon oil as the carrier lipid. This effect can be attributed to the fact that lemon oil does not contain triacylglycerol components and therefore cannot form free fatty acids after digestion. The fact that a small amount of β-carotene (≈5%) could still be solubilized in the micelle phase after digestion for the pure lemon oil system may have been due to the presence of micelles formed by the bile salts and phospholipids present in the simulated intestinal fluids. Micelles formed in the absence of free fatty acids are capable of solubilizing lipophilic bioactive components, but their structures and solubilization capacities will be different from the mixed micelles formed from FFAs, MAGs, bile salts and phospholipids.

Further information about the nature of the association colloids formed in the micelle phase after digestion was obtained by dynamic light scattering (FIG. 7). The nanoemulsions were passed through the simulated GIT model and then centrifuged. The micelle phase was then collected and passed through a 450 nm filter to mimic passage through the mucous layer, which has a pore size around this value. The particle size data was then represented as either intensity-weighted (FIG. 7a) or volume-weighted (FIG. 7b) particle concentrations versus particle diameter. The intensity-weighted concentrations are very sensitive to the presence of any large particles, whereas the volume-weighted concentrations are more sensitive to the presence of smaller particles (such as micelles). The intensity-weighted data showed that there was an appreciable amount of large particles present within the micelle phase, which was attributed to the presence of undigested oil droplets and/or vesicles formed from the digestion products (FIG. 7a). The mean intensity-weighted particle diameter (d_{Z}) increased with decreasing corn oil concentration (FIG. 8), which suggests that these large particles may have been undigested oil droplets since lemon oil is not digested. The volume-weighted data demonstrated that most of the particles present in the micelle phase were actually relatively small, and that there size depended strongly on oil phase composition (FIG. 7b). In this case, there was a decrease in the mean volume-weighted particle diameter (d_{V}) with decreasing corn oil concentration (FIG. 8). These small particles were likely micelles and other small colloidal structures formed from bile salts, phospholipids, free fatty acids, and/or monoacylglycerols. In the absence of corn oil, the micelles are relatively small since they only contain bile salts and phospholipids. However, in the presence of increasing amounts of corn oil the size of the micelles increases due to the incorporation of long chain FFAs and MAGs. In addition, other colloidal structures may be formed (such as vesicles) that may be capable of solubilizing β-carotene.

Conclusions

β-carotene nanoemulsions (d<150 nm) were formed by high pressure homogenization using sucrose monoester and lysolecithin as emulsifiers, and mixtures of corn oil (digestible) and lemon oil indigestible) as the lipid phase. All of the nanoemulsions underwent extensive droplet aggregation under mouth, stomach, and small intestine conditions. The extent of free fatty acid production in the small intestine increased as the amount of digestible oil in the droplets increased. The bioaccessibility of β-carotene also increased with increasing digestible oil content, ranging from ≈5% for the pure lemon oil system to ≈76% for the pure corn oil system. This effect was attributed to the ability of mixed micelles formed from triglyceride digestion products (free fatty acids and monoglycerides) to solubilize β-carotene.

Carrier oil composition (ratio digestible to indigestible oil) influenced the physical stability, microstructure, and bioaccessibility of β-carotene enriched nanoemulsions using a simulated gastrointestinal tract model. The nanoemulsions were stabilized using sucrose monopalmitate (SMP) and lysolecithin as emulsifier, corn oil as digestible oil, and lemon oil as indigestible oil. Carrier oil composition had an appreciable impact on the physicochemical stability of the nanoemulsions within the simulated GIT. Increasing amounts of droplet aggregation were observed in all the nanoemulsions as they passed through the mouth, stomach, and small intestine, which were attributed to changes in interfacial and lipid composition and structure. The amount of free fatty acids produced during lipid digestion and β-carotene bioaccessibility increased as the corn oil content within the oil droplets increased. Indeed, a linear relationship was observed between the amount of FFA formed and β-carotene bioaccessibility. This effect was attributed to an increase in the number of mixed micelles formed capable of solubilizing the β-carotene. In addition, some of the β-carotene may have been trapped within the non-digested lemon oil phase or may have precipitated as crystals in the sediment phase.

### Example 2

### Droplet Size and Composition of Nutraceutical Nanoemulsions Influences Bioavailability of Long Chain Fatty Acids and Coenzyme Q10

The influence of emulsion composition and structure on the gastrointestinal fate of ingested fatty acids and lipophilic bioactive components was examined using an in vivo animal (rat) feeding model. Heptadecanoic (C_{17:0}) acid was selected as a model fatty acid since it is not normally found in the animal's body, and therefore an increase in its concentration in small intestine tissues is a measure of its absorption. Coenzyme Q10 (ubiquinone, CoQ10) was used as an example of a model lipophilic nutraceutical. This compound is known to play an important role in the mitochondrial respiratory chain and is an important biological antioxidant. A number of chronic diseases have been associated with deficiency of CoQ10, including congestive heart failure. Consequently, increasing the intake of CoQ10 by fortification or supplementation is of great interest. The aim was to provide information that would be useful for the development of emulsion-based delivery systems designed to increase the oral bioavailability of lipophilic nutraceuticals.

Materials: Lipase (porcine pancreas, type II, L3126, Batch #096K0747), bile extract, hexane, sodium acetate anhydrous, calcium chloride and Tween 80, were purchased from Sigma-Aldrich (St. Louis, Mo.). Co-enzyme Q10 (CoQ10) and co-enzyme Q6 (CoQ6) were purchased from NuSci (Walnut, Calif.) and Avanti Polar Lipids, Inc. (Alabaster, Ala.), respectively. Heptadecenoic and tridecenoic acids were purchased from Nu-Chek Prep Inc. (Elysian, Minn.). Mineral oil and other chemicals and solvents were obtained from Themo-Fisher Scientific (Pittsburgh, Pa.). Corn oil was purchased from a local grocery store.

Emulsion Formation

An oil phase was prepared by dispersing 0.1% (w/w) CoQ10 and 0.1% (w/w) heptadecanoic acid into carrier lipid (corn oil or mineral oil) and then sonicating for 1 minute and applying mild heating (<50° C. for 5 min) so that complete dissolution was achieved. An aqueous phase containing surfactant was prepared by dispersing 3% (w/w) Tween 80 in aqueous buffer solution (5 mM phosphate buffer, 0.01% (w/w) sodium azide, pH 7.0). A coarse emulsion was prepared by blending 30% (w/w) oil phase and 70% (w/w) aqueous phase together using a high-shear mixer at 20,000 rpm for 4 min. Medium and fine emulsions containing corn oil were then obtained by passing the coarse emulsion once though a 2-stage homogenizer working at 1,000 psi or three-times through a microfluidizer (Model 101, Microfluidics, Newton, Mass.) working at 12,000 psi. Fine emulsions prepared with mineral oil were passed four times through a microfluidizer (Model 101, Microfluidics, Newton, Mass.) at 12,000 psi.

Emulsion Characterization

The mean particle size and electrical charge of the droplets in the emulsions were measured. The particle size distribution was determined by static light scattering (Mastersizer 2000, Malvern Instruments, Worcestershire, UK). Samples were diluted in 10 mM phosphate buffer (pH 7.0) to avoid multiple scattering effects, and then stirred in the dispersion unit of the instrument at a speed of 1250 rpm to ensure they were homogeneous prior to analysis. The particle size was reported as either the surface-weighted mean diameter (d₃₂) or volume-weighted mean diameter (d₄₃).

The ζ-potential of the particles was measured by phase-analysis light scattering (Zetasizer NanoZS, Malvern Instruments, Worcestershire, UK). Samples were diluted 1:10 with 10 mM phosphate buffer solution (pH 7.0) and then placed in a capillary cell equipped with two electrodes to assess the electrophoretic mobility of the particles.

In Vitro Digestion

The digestion of the lipid droplets under simulated small intestinal conditions was monitored using a pH-stat device (835 Titrando, Metrohm, Riverview, Fla.). Emulsions were diluted 1:30 prior to in vitro digestion with 10 mM phosphate buffer (pH 7.0) to achieve a final oil concentration of 1% (w/w). An aliquot of 30 mL of the resulting emulsion was placed in a temperature-controlled (37° C.) chamber and the pH was adjusted to 7.0 using NaOH solution (0.25 M). Then, 4 mL of bile extract (46.87 mg/mL) and 1 mL of calcium chloride (110 mg/mL) solutions dissolved in phosphate buffer were added to the sample chamber, and the pH was adjusted back to 7.0 if necessary. Then, 2.5 mL of freshly prepared lipase suspension (24 mg/mL) dissolved in phosphate buffer was added to the mixture. The pH of the mixture was monitored and the volume of NaOH (mL) necessary to neutralize the free fatty acids (FFA) released due to lipid digestion so as to maintain the pH at 7.0 was recorded during 2 h. The following equation was used to calculate the percentage of FFAs released during the digestion process, assuming that each molecule of triacylglycerol (TAG) generates two molecules of FFA when completely digested:

$$FFA ( t ) = 100 ( \frac{{V}_{NaOH} ( t ) \cdot {C}_{NaOH} \cdot {M}_{Oil}}{2 \cdot {m}_{Oil}} ) \qquad ( 2 )$$

where V_{NaOH}(t) is the volume of NaOH solution required to neutralize the FFAs produced at digestion time t (L), C_{NaOH} is the molarity of the NaOH solution used to titrate the sample (mol L⁻¹), Mₒᵢₗ is the molecular weight of the oil (g mol⁻¹), and mₒᵢₗ is the total mass of oil initially present in the incubation cell (g). The molecular weight of the corn oil was considered to be 800 g mol⁻¹. The in vitro digestion experiments were carried out in triplicate and the mean and standard deviation were calculated from this data.

In Vivo Experiments

An animal feeding study was used to test the bioavailability of free fatty acids and CoQ10. Female Sprague Dawley rats (about 200-250 g) were purchased from Charles River Laboratories (Wilmington, Mass.). The Institutional Animal Care and Use Committee of the University of Massachusetts, Amherst, approved all animal procedures. Animals were housed in individual wire-bottomed cages in a windowless room with a 12-h light-dark cycle. After 10 days of adaptation, rats were fasted for 6 hours and 5 ml of emulsion was administered into a rat's stomach using a feeding needle (Roboz Surgical Instrument, Gaithersburg, Md.). Sample administration was conducted five times with 30 min intervals. Thirty min after the final administration, the rats were sacrificed by CO₂ gas asphyxiation and blood samples were collected via cardiac puncture. Collected blood samples were allowed to clot for 30 min and then the serum layer was separated by centrifugation at 800 g for 20 min at 4° C. Serum was kept at −80° C. until analysis. The small intestine was flushed with phosphate buffered saline (PBS) five times, and the duodenum, jejunum, and ileum were collected. The intestine samples were kept at −80° C. until fatty acid composition and CoQ10 analyses. The in vivo experiment was carried out once, using three rats per sample analyzed.

CoQ10 Analysis

CoQ10 stock solution was prepared by dissolving 20 mg in 100 mL 1-propanol and stock solution was diluted to obtain various concentration of standard solution (0, 50, 100, 200 μg/mL). CoQ6 was dissolved in 1-propanol (200 μg/mL) and used as an internal standard.

Intestine extract was prepared by a method described previously by Tang, P. H., Miles, M. V., Miles, L., Quinlan, J., Wong, B., Wenisch, A., and Bove, K., “Measurement of reduced and oxidized coenzyme Q(9) and coenzyme Q(10) levels in mouse tissues by HPLC with coulometric detection”, Clinica Chimica Acta, 341(1-2):173-84 (2004), which is incorporated herein in its entirety. After thawing the intestine, approximately, 50 mg of intestine was weighed and 1 ml of ice cold 0.15M NaCl solution was added. Intestine sample (about 50 mg) was homogenized on ice and 50 μL of CoQ6 solution (Internal standard) and 25 μL of 1,4-benzoquione (5 mg/mL) solution was added to oxidize CoQ10. After 30 min. incubation at room temperature, 5 ml of 1-propanol was added and then mixed by vortexing for 1 min. Sample was centrifuged at 2,000 g for 5 min and the supernatant was filtered with a 0.1 μm disposable syringe filter (Celltreat, Shirley, Mass.). The oxidized CoQ10 concentration was standardized relative to the tissue weight.

Quantitative analysis was carried out using a high performance liquid chromatography system (Shimadzu Co. Japan) equipped with a pump (LC-10 AT), UV-visible detector (SPD-M10A), and controller (SCL-10A). A reverse phase C-18 column (ODS-2 Hypersil, 200×4.6 mm, 5 μm, Thermo scientific, Waltham, Mass.) was used. The mobile phase comprised of a mixture of 4.2 g sodium acetate anhydrous, 15 mL of glacial acetic acid, 15 mL 2-propanol, 830 mL methanol, and 140 ml hexane. The injection volume was 100 μl, the flow rate was 1 ml/min, UV detection was made at 275 nm and all analysis was performed at room temperature under isocratic conditions.

Heptadecanoic Acid Analysis

Lipids were extracted from samples (duodenum, jejunum, ileum) and blood using a standard method by Folch, J., Lees, M., and Stanley, G. H. S., “A simple method for the isolation and purification of total lipids from animal tissues”, Journal of Biological Chemistry, 226(1):497-509 (1957), which is incorporated herein in its entirety, and then the fatty acids were methylated prior to gas chromatography analysis as taught by Park, Y. and Pariza, M. W., “Evidence that commercial calf and horse sera can contain substantial amounts of trans-10,cis-12 conjugated linoleic acid”, Lipids, 33(8):817-19 (1998), which is incorporated herein in its entirety. Tridecenoic acid was used as an internal standard. Methyl esters of fatty acids were analyzed using a GC instrument (GC-17A, Shimadzu, Kyoto, Japan) fitted with a flame-ionization detector. A fused-silica capillary column (30 m×0.25 mm i.d., 0.25 mm film thickness) was used (DB-5, Agilent Technologies, Wilmington, Del.) and the oven temperature was programmed to be held for 4 min at 150° C., increased by 4° C. per min to 250° C., and then held for 5 min. Tridecanoic acid was used as an internal standard, and absorbed heptadecanoic acid was calculated relative to the tissue weight.

Statistical Analysis

The results were analyzed using a software program (JMP 8, SAS Institute Inc.) to perform analysis of variance. The Student's t test was run to determine significant differences. In vivo experiments were carried out using three rats per treatment and the data were analyzed with the Statistical Analysis System (SAS Institute, Cary, N.C.). Significant differences between treatments were determined using the GLM procedure. Significant differences were defined at P<0.05.

Results and Discussion

Properties of Initial Emulsions

The particle size and charge was measured for the various emulsions used in the in vitro and in vivo studies (Table 3). Emulsions were prepared from digestible oil (corn oil) with a range of different mean particle diameters, i.e., d₃₂=0.21, 0.70, and 2.27 μm. This range covers the size of the droplets typically found in many emulsion-based food products, such as soft drinks, milk, cream, dressings and sauces. All of the droplets in these emulsions had fairly similar negative charges (around −7.5 mV). The droplets were stabilized by a non-ionic surfactant (Tween 80), and may therefore have been expected to be neutral. Nevertheless, a negative charge on lipid droplets coated by non-ionic surfactants, may be attributed to the presence of anionic impurities in the surfactant or oil phases, such as free fatty acids or phospholipids. Fine emulsions were prepared from indigestible oil (mineral oil) that had a similar mean diameter (0.21 μm) as the fine emulsions prepared using the digestible oil (corn oil). These emulsions also had some negative charge (around −9.0 mV), which may be due to impurities in the oil and/or surfactant components used to fabricate them.

TABLE 3
Composition and physicochemical properties of different emulsions
used in in vitro and in vivo studies. (A) large; (B) medium;
(C) small; (D) small size with mineral oil. Mean with different
letters (a, b, c) are significantly different (p < 0.05)

| | Oil | d₃₂ | d₄₃ | ζ-potential |
|---|---|---|---|---|
| Sample | Composition | (μm) | (μm) | (mV) |
| | Corn Oil | 2.27 ± 0.03 ^{a} | 11.87 ± 0.04 ^{a} | −7.4 ± 0.4 |
| | Corn Oil | 0.70 ± 0.02 ^{b} | 2.18 ± 0.03 ^{b} | −7.5 ± 0.3 |
| | Corn Oil | 0.21 ± 0.01 ^{c} | 0.26 ± 0.01 ^{c} | −7.8 ± 0.2 |
| | Mineral Oil | 0.21 ± 0.01 ^{c} | 0.26 ± 0.01 ^{c} | −9.0 ± 0.1 |

In Vitro Studies

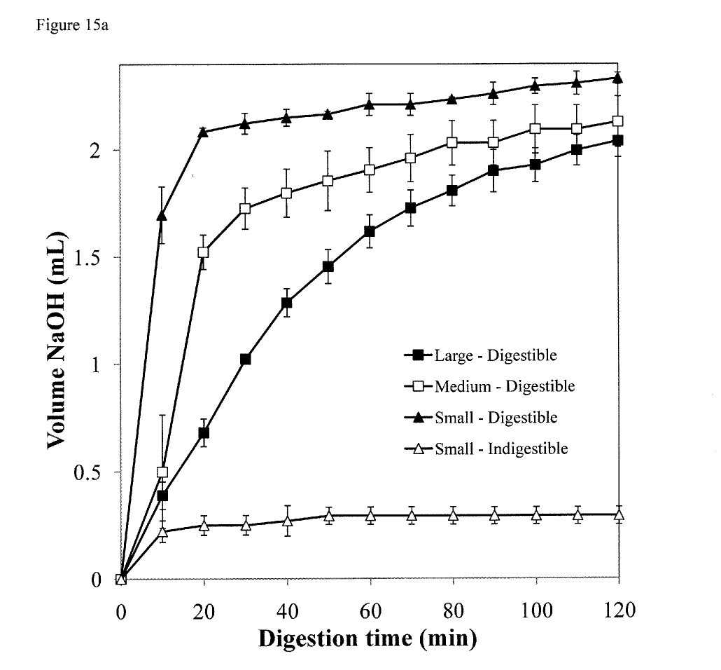

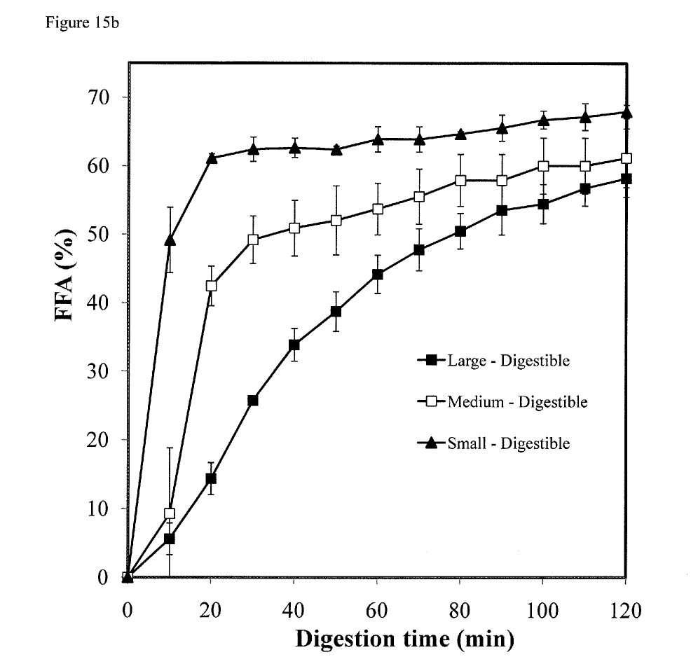

The rate and extent of lipid digestion was measured using a simulated small intestine model (pH stat method) for the four emulsions: large, medium and small digestible emulsions and small indigestible emulsions. The volume of NaOH titrated into the samples to maintain a constant pH (7.0) was measured as a function of digestion time (FIG. 15a), and then the fraction of free fatty acids released from the emulsions containing digestible oil was calculated after subtraction of the values from the emulsions containing indigestible oil (FIG. 15b). For the emulsions prepared using indigestible oil (mineral oil) there was a slight increase in the volume of NaOH added to maintain constant pH within the initial 10 minutes, but then the pH did not change appreciably afterwards. This initial increase could be attributed to the presence of other components in the reaction chamber that may be digested by the simulated intestinal fluids, such as phospholipids or proteins. Therefore, the values for the indigestible oil were subtracted from the values obtained for the digestible oil to obtain the amount of FFA released. For all the emulsions prepared using digestible oil (corn oil), there was a steep increase in the amount of FFA released initially, followed by a more gradual increase at longer times, until a relatively constant final value was reached. However, there were appreciable differences between the rate and extent of digestion depending on the initial droplet size. The initial rate of digestion was faster, and the final amount of FFAs released after 2 hours of incubation increased as the particle size decreased. The amount of FFAs produced per unit time increases as the droplet size decreased could be attributed to changes in the surface area of oil exposed to the digestive enzymes. Other factors may also contribute to the overall dependence of digestion rate on initial droplet size, including the concentration of free surfactant remaining in the aqueous phase (since this can inhibit lipase adsorption) and changes in interfacial structure with droplet size (since this can affect the ability of lipase to interact with the oil phase).

Particle size and digestibility will influence the subsequent bioavailability of encapsulated lipophilic nutraceuticals. First, bioactive components may become trapped within non-digested lipid fraction and not be released. Second, the free fatty acids (FFAs) and monoacyglycerols (MAGs) produced by lipid digestion can form mixed micelles that can solubilize and transport lipophilic components to the enterocytes where they are absorbed. Third, FFAs and MAGs are reassembled within enterocytes to form chylomicrons that transport lipophilic nutraceuticals into the lymphatic system, and then into the systemic circulation. Thus, if the amount of digestion products produced is reduced, the solubilization capacity of the intestinal fluids for the bioactive components will be decreased. As mentioned earlier, in vitro digestion methods cannot accurately simulate the highly dynamic, compositionally complex, and structurally diverse environment of the gastrointestinal tract. Therefore the influence of emulsion structure and composition on the bioavailability of the lipophilic components was examined using an in vivo model.

In Vivo Studies

Two lipophilic compounds were incorporated into the oil phase prior to formation of the emulsion-based delivery systems: heptadecanoic acid and co-enzyme Q10. Heptadecanoic acid is an odd-carbon fatty acid that is present at very low levels in animal tissues, and can therefore be used as a fatty acid marker for absorption studies. Co-enzyme Q10 is a lipophilic nutraceutical that participates in cell respiration and is known to be absorbed along with lipid-soluble components in the small intestine. The bioavailability of the bioactive components was then determined using an animal (rat) model.

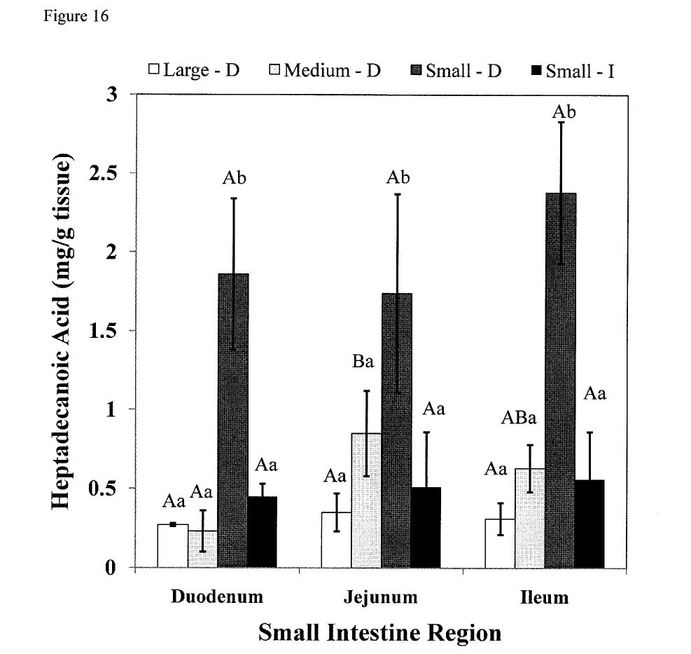

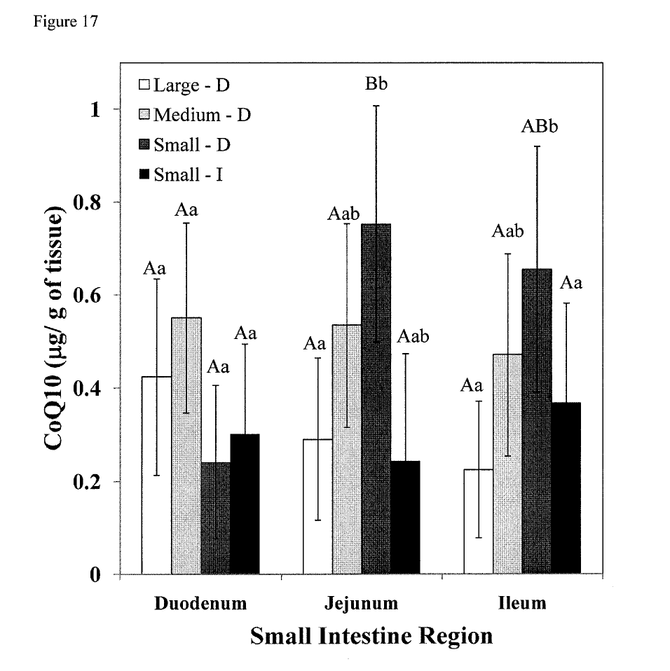

The results indicate that the absorption of heptadecanoic acid from the small digestible emulsion was significantly higher (P>0.05) than from the medium or large digestible emulsions, or from the small indigestible emulsions, in all regions of the small intestine, i.e., duodenum, jejunum, and ileum (FIG. 16). Similarly, the absorption of CoQ10 from the small digestible emulsion was significantly higher than from the large and medium digestible emulsions and from the indigestible small emulsions in the jejunum and ileum. However, no significant differences (P<0.05) in CoQ10 absorption were observed in the duodenum. These results suggest that emulsion-based delivery systems containing small digestible lipid droplets may be effective at increasing the oral bioavailability of lipophilic nutraceuticals. The origin of this effect may be attributed to the fact that small oil droplets are digested more rapidly, thereby releasing more of the heptadecanoic acid. In addition, the generation of lipid digestion products (FFAs and MAGs) would have occurred more rapidly for small digestible oil droplets, which would facilitate the solubilization and transport of the lipophilic nutraceuticals to the enterocytes, as well as stimulating the formation of chylomicrons within the enterocytes, which in turn can enhance the absorption of lipophilic nutraceuticals. Interestingly, there appeared to be slightly more absorption of the heptadecanoic acid from the small indigestible droplets than from the medium or large digestible droplets in the duodenum and jejunum, even though the differences were not significant (FIG. 16). In addition, there appeared to be some absorption of the CoQ10 from the small indigestible droplets in all regions of the small intestine (FIG. 17). These results suggest that lipophilic bioactive molecules may have been able to move out of the indigestible oil droplets and be absorbed by the epithelium cells. Alternatively, it may be possible for entire small indigestible droplets to be absorbed by epithelium cells.

Conclusions

The influence of droplet size (d₃₂=0.21, 0.70 or 2.2 μm) and oil digestibility (corn oil versus mineral oil) on the bioavailability of a model long chain fatty acid (heptadecanoic acid) and lipophilic nutraceutical (Coenzyme Q10) was investigated using a rat feeding study. Small droplets were digested more rapidly than large droplets using a simulated small intestinal model (pH stat), which was attributed to the greater surface area of lipid exposed to intestinal juices. The pH stat model also confirmed that emulsified corn oil was digestible, whereas emulsified mineral oil was indigestible. A rat feeding study showed that the bioavailability of the fatty acid and lipophilic nutraceutical in small intestinal tissues was highest when they were encapsulated within digestible oil droplets with the smallest size.

In summary the size and composition of the droplets in emulsion-based delivery systems influences the rate and extent of lipid digestion, as well the bioavailability of lipophilic bioactive components: Coenzyme Q10 and heptadecanoic acid. An in vitro digestion model showed that the rate of lipid digestion increased as the droplet size decreased, which was attributed to the increase in surface area of lipid exposed to intestinal juices containing lipase. An in vivo digestion model (rat feeding study) showed that the bioavailability of a model fatty acid (heptadecanoic acid) and lipophilic bioactive (Coenzyme Q10) agent in small intestine tissues was highest when they were encapsulated with digestible (corn oil) droplets with the smallest size.

### Example 3

### Increase of Curcumin Bioaccessibility Using Excipient Emulsions

In this specific example of the present teachings, excipient emulsions were prepared as a means to increase the bioaccessibility of powdered curcumin. Crystalline forms of lipophilic bioactive agents usually have a much lower bioavailability than solubilized forms. Therefore powdered curcumin was mixed with an excipient emulsion to try to increase its bioaccessibility due to its ability to increase the solubility of curcumin in the intestinal fluids. The effect of incubation temperature and time on the transfer of curcumin into the excipient emulsion prior to ingestion was measured, as well as the bioaccessibility of curcumin after exposure to a simulated gastrointestinal tract (GIT). Two incubation temperatures (30 and 100° C.) were used to mimic conditions that curcumin might experience in food applications: (i) within ambient foods (such as salads); (ii) within cooked foods (such as curry sauces).

Materials: Corn oil purchased from a local supermarket was used as an example of a digestible long chain triglyceride. The following chemicals were purchased from the Sigma Chemical Company (St. Louis, Mo.): curcumin (SLBH2403V), mucin from porcine stomach (SLBH9969V), pepsin from porcine gastric mucosa (SLBL1993V), lipase from porcine pancreas pancreatin (SLBH6427V), porcine bile extract (SLBK9078), Tween 80 (BCBG4438V), and Nile Red (063K3730V). The supplier reported that the activity of the pepsin was around 250 units/mg, and the activity of the lipase was around 100-400 units/mg protein (using olive oil). All other chemicals were of analytical grade. Double distilled water was used to prepare all solutions and emulsions.

Excipient emulsion preparation. Initially, an aqueous phase was prepared by mixing 1% (w/w) Tween 80 with an aqueous buffer solution (10.0 mM phosphate buffer saline (PBS), pH 6.5). Coarse oil-in-water emulsions were prepared by homogenizing 10% (w/w) corn oil with 90% (w/w) aqueous phase using a high-speed blender for 2 min (M133/1281-0, Biospec Products, Inc., ESGC, Switzerland). Fine emulsions were then obtained by passing the coarse emulsions through a microfluidizer (M110Y, Microfluidics, Newton, Mass.) with a 75 μm interaction chamber (F20Y) at an operational pressure of 9,000 psi for 5 passes. The resulting excipient emulsions were stored in a refrigerator at 4° C. before use.

Preparation of curcumin-emulsion, curcumin-oil, and curcumin-buffer mixtures. Curcumin (3 mg) was weighed into a beaker and then excipient emulsion (10 mL) was added. The resulting mixtures were then incubated at either 30 or 100° C. for different times (ranging from 10 min to 120 min for 30° C. and from 10 min to 60 min for 100° C.). After incubation, selected samples were immediately placed in an ice water and then used for the following experiments. In some experiments, the curcumin was mixed with bulk corn oil or with buffer (PBS) solution rather than an emulsion.

Curcumin solubility in mixtures. The solubility of curcumin in each mixture was measured spectrophotometrically based on the method of Ahmed, K., Li, Y., McClements, D. J. and Xiao, H., “Nanoemulsion- and emulsion-based delivery systems for curcumin: Encapsulation and release properties”, Food Chemistry, 132(2):799-807 (2012), which is incorporated herein in its entirety, with some modifications. 10 mL of mixture was collected, and then centrifuged at 1750 rpm for 10 min at ambient temperature (CL10 centrifuge, Thermo, Scientific, Pittsburgh, Pa., USA) to remove non-dissolved (crystalline) curcumin. 1 mL of supernatant was then mixed with 5 mL chloroform, vortexed, and centrifuged at 1750 rpm for 10 min at ambient temperature. The bottom layer containing the solubilized curcumin was collected, while the top layer was mixed with an additional 5 mL of chloroform and the same procedure was repeated. The two bottom chloroform layers were combined, and diluted to an appreciate concentration to be analyzed by a UV-VIS spectrophotometer at 419 nm (Ultraspec 3000 pro, GE Health Sciences, USA). A cuvette containing pure chloroform was used as a reference cell. The concentration of curcumin extracted from each mixture was calculated from a calibration curve of absorbance versus curcumin concentration in chloroform. The solubility of curcumin in each mixture was then calculated as the concentration of curcumin extracted from each mixture multiplied by the dilution factor.

Particle characterization. The mean particle diameters (Z-average) and particle size distributions of the curcumin-emulsion mixtures after incubation at different temperatures were monitored using a dynamic light scattering instrument (Nano-ZS, Malvern Instruments, Worcestershire, UK). The electrical charge (ζ-potential) of the particles in these samples was measured using a micro-electrophoresis instrument (Nano-ZS, Malvern Instruments, Worcestershire, UK). Samples were diluted with buffer solution (10.0 mM PBS, pH 6.5) prior to measurements to avoid multiple scattering effects.

The mean particle diameter and particle size distribution of samples exposed to simulated gastrointestinal conditions was measured using static light scattering (Mastersizer 2000, Malvern Instruments Ltd., Worcestershire, UK). Samples were diluted with appropriate buffer solutions (same pH as GIT phase) and stirred in the dispersion unit at a speed of 1200 rpm. The particle size is reported as the surface-weighted mean diameter (d₃₂).

Microstructural analysis. The microstructure of samples was characterized using confocal scanning fluorescence microscopy (Nikon D-Eclipse C1 80i, Nikon, Melville, N.Y.). Samples analyzed by confocal microscopy were dyed with Nile Red to highlight the location of the lipid phase. The Nile red was dissolved in absolute ethyl alcohol at a concentration (1 mg/mL). Then, before analysis 2 mL emulsion samples were mixed with 0.1 mL Nile Red solution (1 mg/mL ethanol) to dye the oil phase. All images were captured with a 10× eyepiece and a 60× objective lens (oil immersion). Changes in the properties of curcumin crystals in the mixtures were observed using a cross-polarized lens (C1 Digital Eclipse, Nikon, Tokyo, Japan).

Curcumin oil-solubility characteristics. The temperature-dependence of the dissolution of crystalline curcumin into bulk corn oil was characterized by measuring the change in turbidity (600 nm) with temperature using a UV-visible spectrophotometer equipped with a temperature controller (Agilent Cary 200, Agilent, Santa Clara, Calif.). This method can be used to detect the presence of curcumin crystals in the oil phase and indirectly determine the solubility of curcumin in bulk corn oil. A weighed amount of curcumin (3 or 4 mg/mL) was dispersed in corn oil at ambient temperature, and then the mixture was heated from 25 to 100° C., and then cooled from 100 to 25° C. at a rate 1° C./min with continuous stirring. In some experiments, the change in turbidity with time was measured at a fixed incubation temperature (30 or 100° C.) to determine the kinetics of isothermal dissolution.

Simulated gastrointestinal digestion: Powdered curcumin was mixed with excipient emulsion, corn oil, or buffer solution and then held at 30° C. for 30 min or 100° C. for 10 min. Each sample was passed through a three-step GIT model that consisted of mouth, gastric, and small intestine phases.

Initial system: The initial systems were placed into a glass beaker in an incubator shaker at a rotation speed of 100 rpm for 15 min at 37° C. for preheating (Innova Incubator Shaker, Model 4080, New Brunswick Scientific, New Jersey, USA). Three different initial systems were tested: (i) curcumin and excipient emulsion; (ii) curcumin, corn oil, and buffer solution; or, (iii) curcumin and buffer solution. The initial concentration of curcumin was the same in all systems, while the initial concentration of corn oil in systems (i) and (ii) were the same.

Mouth phase: A simulated saliva fluid (SSF) containing 3 mg/mL mucin and various salts was prepared as described previously in Mao, Y., McClements, D. J., “Influence of electrostatic heteroaggregation of lipid droplets on their stability and digestibility under simulated gastrointestinal conditions”, Food & Function, 3(10):1025-34, (2012), which is incorporated herein in its entirety. SSF was preheated to 37° C. and then mixed with the preheated curcumin mixture at a 1:1 mass ratio. The mixture was then adjusted to pH 6.8 and placed in an incubator shake at 100 rpm and 37° C. for 10 min. This incubation time is longer than a food would normally spend in the mouth, but was used to minimize sample-to-sample variations that might occur if very short times were used.

Stomach phase: Simulated gastric fluid (SGF) was prepared by placing 2 g NaCl and 7 mL HCl into a container, and then adding double distilled water to 1 L. The bolus sample from the mouth phase was then mixed with SGF containing 0.0032 g/mL pepsin preheated to 37° C. at a 1:1 mass ratio. The mixture was then adjusted to pH 2.5 and placed in a shaker at 100 rpm and 37° C. for 2 hours to mimic stomach digestion. Gastric lipase was not included in the SGF because of the difficulty in obtaining a reliable and economically viable source of this digestive enzyme. Gastric lipase typically promotes a limited amount of lipid digestion within the stomach phase, and therefore its omission should only have a fairly modest impact on the gastrointestinal fate of the excipient emulsions.

Small Intestine phase: 30 mL chyme samples from the stomach phase were diluted with buffer solution (10 mM PBS, pH 6.5) to obtain a final corn oil concentration of 1.25%. The diluted chyme was then incubated in a water bath (37° C.) for 10 min and then the solution was adjusted back to pH 7.0. Next, 3 mL of simulated intestinal fluid (containing 0.5 M CaCl₂ and 7.5 M NaCl) was added to 60 mL digesta. Then, 7 mL bile extract, containing 375.0 mg bile extract (pH 7.0, PBS), was added with stirring and the pH was adjusted to 7.0. Finally, 5 mL of pancreatic suspension, containing 120 mg of lipase (pH 7.0, PBS), was added to the sample and an automatic titration unit (Metrohm, USA Inc.) was used to monitor the pH and control it to a fixed value (pH 7.0) by titrating 0.25 M NaOH solution into the reaction vessel for 2 h at 37° C. The percentage of free fatty acids released in the sample was calculated from the number of moles of NaOH required to maintain neutral pH as described previously (27). Some of the free fatty acids may not be fully ionized at pH 7, and therefore the FFAs determined by the pH stat method are only the titrable ones.

Curcumin bioaccessibility. After in vitro digestion, 30 mL raw digesta of each mixture was centrifuged (18000 rpm, Thermo Scientific, USA) at 25° C. for 30 min. The clear supernatant was collected and assumed to be the “micelle” fraction in which the curcumin was solubilized. In some samples, a layer of non-digested oil was observed at the top of the samples and it was removed from the micelle fraction. Aliquots of 5 mL of micelle fraction were mixed with 5 mL of chloroform, vortexed and centrifuged at 1750 rpm for 10 min at ambient temperature. The bottom layer containing the solubilized curcumin was collected, while the top layer was mixed with an additional 5 mL of chloroform and the same procedure was repeated. The two collected chloroform layers were mixed together, and then diluted to an appreciate concentration to be analyzed by a UV-VIS spectrophotometer at 419 nm. The concentration of curcumin was calculated from the absorbance using a standard curve using a suitable dilution factor. The bioaccessibility of curcumin was calculated according to the following expression: BA %=100×c_{M}/c_{D}, where c_{M} and c_{D} are the curcumin concentrations in the mixed micelle phase and in the total digesta collected after the small intestine phase, respectively.

Statistical analysis. All experiments were carried out on at least two freshly prepared samples. The results are expressed as means±standard deviation (SD). Data was subjected to statistical analysis using SPSS software version 18.0. Differences were considered significant at p<0.05.

Results and Discussion

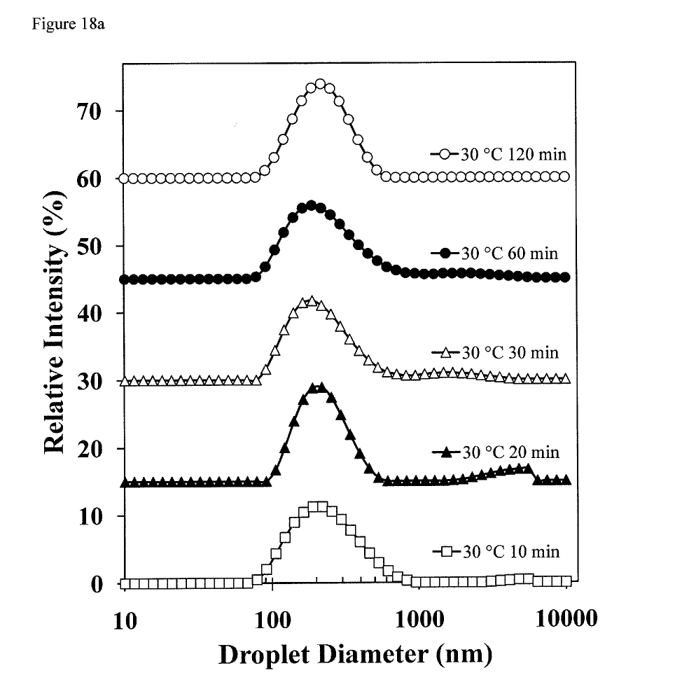

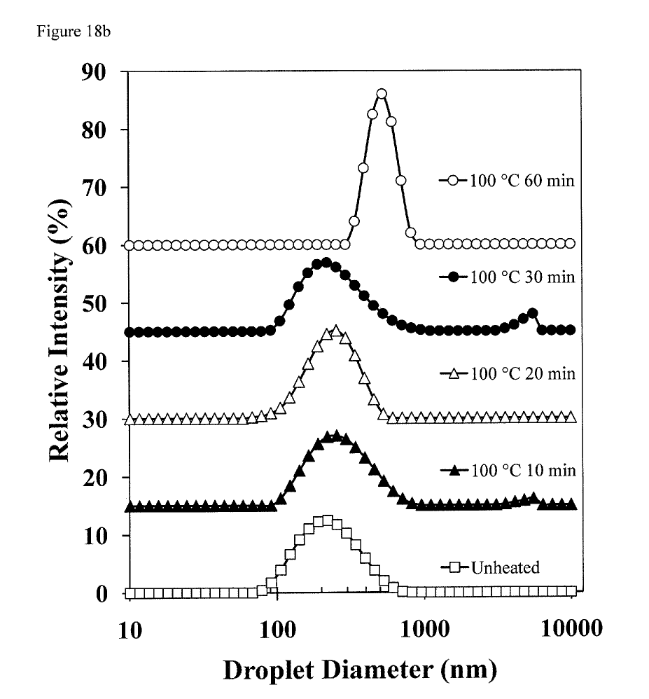

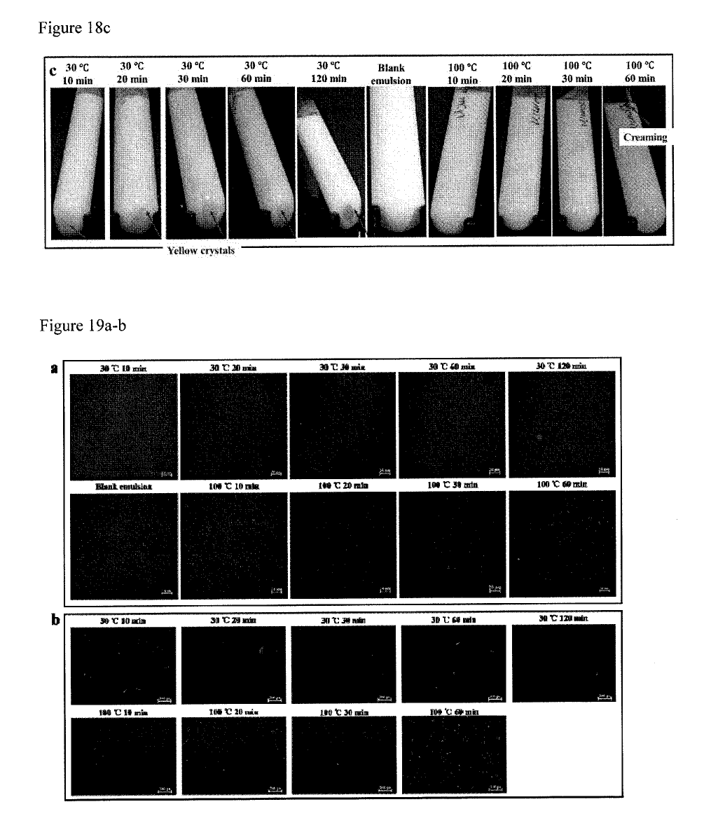

Effect of incubation temperature on particle characteristics. Curcumin-containing samples were exposed to two different incubation temperatures to simulate different conditions that they might experience in food applications: (i) 30° C. for salads at ambient temperature; (ii) 100° C. for sauces at cooking temperatures. The mixture of curcumin and excipient emulsion was stirred to ensure homogeneity, and then incubated at either 30 or 100° C. for different times. The mean particle diameter, particle size distribution, particle charge, and visual appearance of the different samples was then measured (Table 4, FIGS. 18a-c).

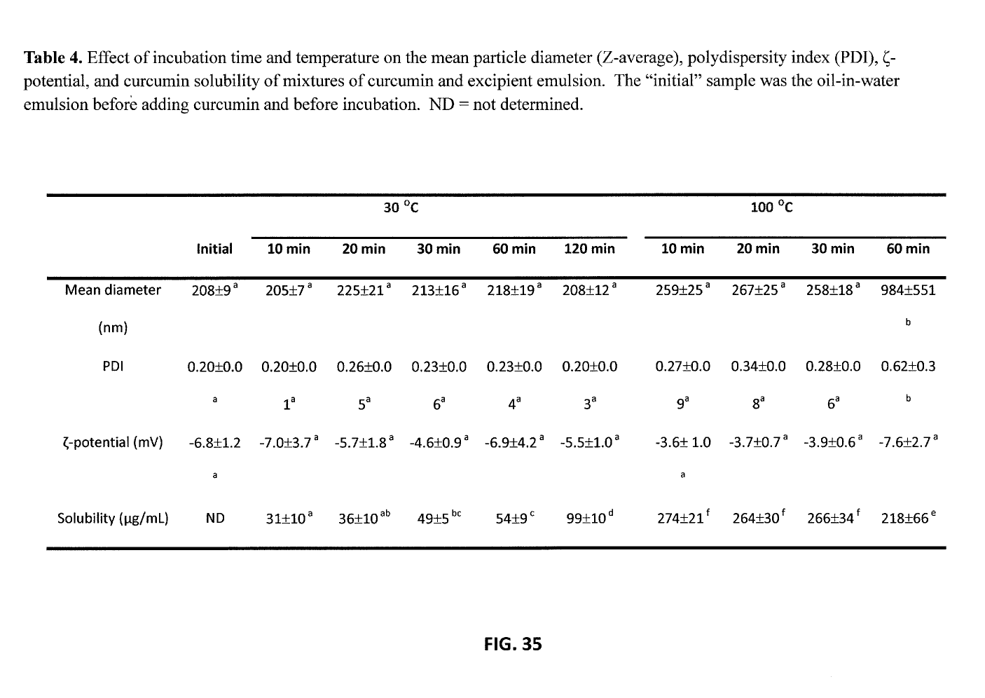

There were no appreciable changes in the characteristics of the droplets in the curcumin-emulsion mixtures during incubation at 30° C. for up to 120 min (Table 4 and FIG. 18a). Indeed, there were no significant changes in the mean particle diameter, polydispersity index (PDI), or ζ-potential of the curcumin-emulsion mixtures when compared to the initial emulsions. The curcumin-emulsion systems were also stable to incubation at 100° C. from 10 to 30 min, with no significant changes in mean particle diameter, PDI, or ζ-potential. However, there was evidence of some droplet creaming in the curcumin-emulsion mixture after heating at 100° C. for 60 min (indicated by an arrow in FIG. 18c). Moreover, there was a significant increase in the mean particle diameter and PDI for these emulsions (FIG. 18b and Table 4 (FIG. 35)). This effect may be due to droplet coalescence resulting from dehydration of the non-ionic surfactant head groups at elevated temperatures. There was no significant change in the magnitude of the electrical charge on the emulsion droplets when they were incubated at either 30 or 100° C., which suggests that the interfacial composition remained relatively constant.

The microstructures of the curcumin-emulsion mixtures after heating at 30 and 100° C. for different times were recorded using confocal fluorescence microscopy (FIG. 19a). There was little change in the microstructure of the mixed systems incubated at 30° C. for different times, confirming their high stability under these conditions. However, there was evidence of some larger oil droplets in the mixed systems after incubation at 100° C. for prolonged times, suggesting that some coalescence had occurred.

The amount of curcumin transferred into the excipient emulsions over time was measured at different incubation temperatures. The amount of curcumin solubilized in the excipient emulsions depended on incubation temperature and time. There was a gradual increase in the amount of curcumin solubilized within the excipient emulsion for mixtures incubated at 30° C. (Table 4). On the other hand, there was a rapid increase in the amount of curcumin solubilized in the excipient emulsions during the first 10 minutes of incubation at 100° C., followed by an appreciable decrease at longer incubation times. This decrease was attributed to the fact that droplet coalescence and oiling off occurred in the emulsions held at the higher temperature, which meant that some of the curcumin remained in the upper oil phase and was not therefore measured. Overall, the amount of curcumin solubilized within the excipient emulsions was considerably higher for the samples incubated at the higher holding temperatures. For example, the amount of curcumin solubilized in the emulsions was around 54 and 218 μg/mL after incubation for 60 min at 30° C. and 90° C., respectively.

The presence of curcumin crystals within the different mixtures after exposure to different incubation temperatures was observed using a crossed polarizer lens (FIG. 19b). Some crystalline material was clearly observed in the curcumin-emulsion mixtures incubated at 30° C. from 10 to 120 min, which suggested that the curcumin crystals did not completely dissolve when held at this temperature in the presence of the excipient emulsion. Conversely, no crystals were observed in the curcumin-emulsion mixtures held at 100° C. at any incubation time, which indicated that curcumin crystals rapidly and completely dissolved within the emulsions at this elevated temperature. However, there was evidence of some large droplets in these samples after heating for prolonged times at 100° C., again suggesting that droplet coalescence occurred.

Temperature dependence of curcumin oil-solubility. The dependence of the transfer of curcumin into the excipient emulsions on incubation temperature and time may be due to changes in its oil solubility with temperature. Therefore turbidity measurements were used to monitor the solubility of curcumin crystals in bulk corn oil at different temperatures. The principle of this method is that the turbidity of the curcumin-oil mixture is relatively high when curcumin crystals are present because they scatter light strongly, but it is relatively low when the crystals have melted or dissolved due to the decrease in light scattering.

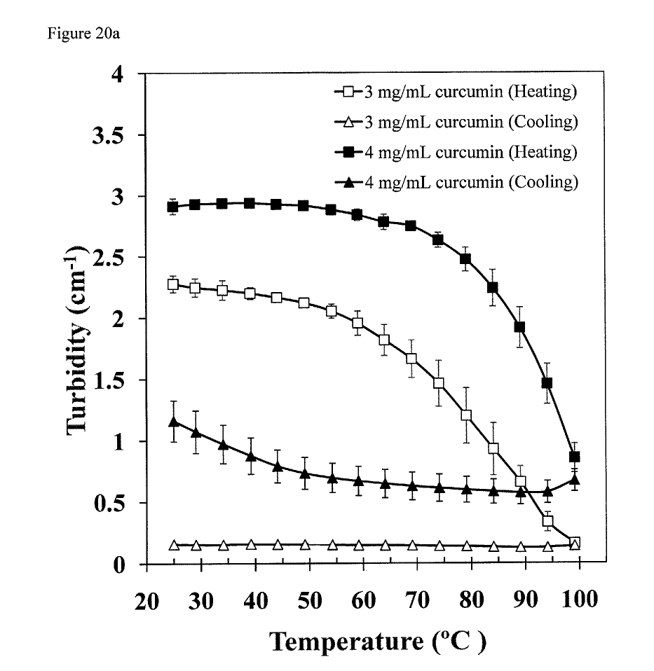

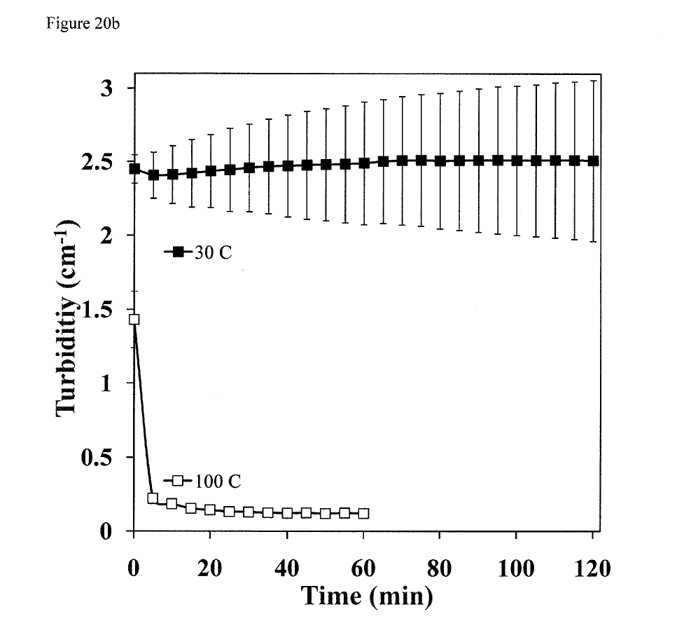

The turbidity of curcumin in corn oil mixtures (3 mg/mL) decreased appreciably upon heating from 25 to 100° C. (FIG. 20a) until it reached a value close to zero at 100° C. indicating that the crystals had fully dissolved at this temperature. Upon cooling, the turbidity stayed low suggesting that the curcumin remained dissolved within the oil. This may have occurred because curcumin was below its saturation temperature at the lower temperatures, or because of supersaturation/supercooling effects. Therefore the change in turbidity with temperature was measured for a curcumin/oil mixture containing a higher curcumin concentration (4 mg/mL). In this case, the turbidity still decreased appreciably with increasing temperature, but the final turbidity reached at high temperatures was considerably greater than that observed at the lower curcumin level. Indeed, the samples at the higher temperatures still appeared turbid, suggesting that not all of the curcumin crystals had dissolved. When this sample was cooled down the turbidity remained relatively high and even increased slightly, which can be attributed to the fact that the solubility of curcumin decreases with decreasing temperature and the amount of curcumin present was above the saturation level. These results suggest that the solubility of curcumin in corn oil at ambient temperature was somewhere between 3 and 4 mg/mL.

Information about the kinetics of curcumin dissolution at the two incubation temperatures was obtained by measuring changes in the turbidity of curcumin/oil mixtures (3 mg/mL) over time (FIG. 20b). There was little change in the turbidity of the mixture held at 30° C. suggesting that curcumin crystals only dissolved slowly at this temperature. On the other hand, the turbidity decreased rapidly in the mixture held at 100° C., which indicated that the curcumin crystals rapidly dissolved in the corn oil at this elevated temperature.

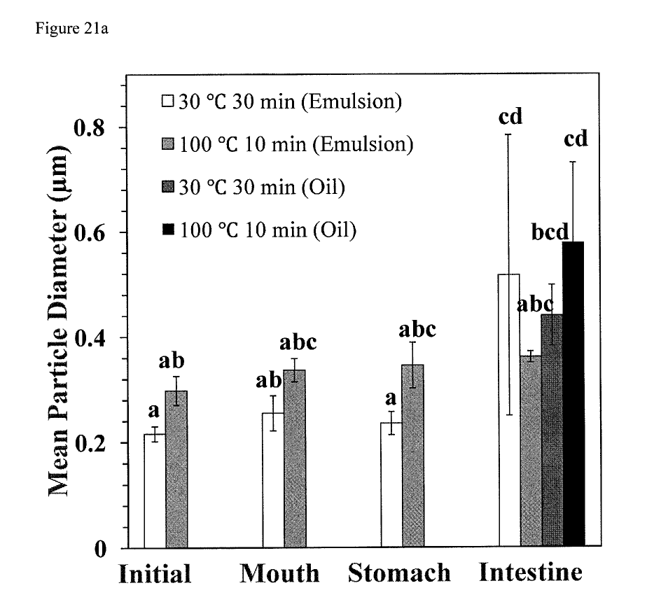

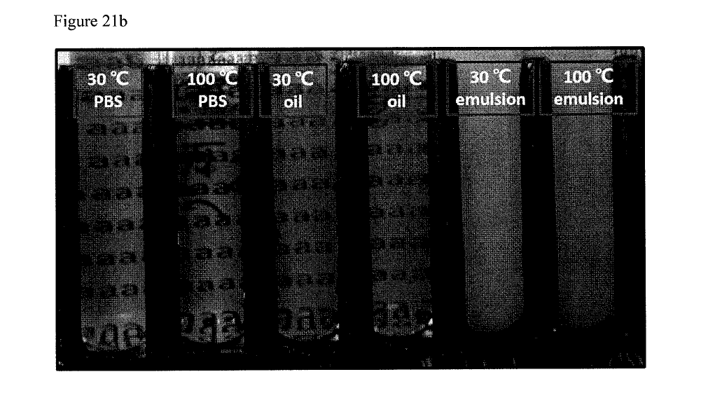

Influence of simulated digestion on particle properties. The influence of the excipient emulsions on the potential biological fate of curcumin was examined using an in vitro gastrointestinal tract (GIT) model that simulates the mouth, stomach, and small intestine phases. Curcumin-emulsion mixtures incubated at 30° C. for 30 min or at 100° C. for 10 min were selected for the GIT study to simulate ambient food applications (such as salad dressings) and cooking applications (such as curry sauces). These incubation times were selected because they led to excipient emulsions that were physically stable, and that might simulate usage conditions. The amount of curcumin solubilized in the excipient emulsions at the higher temperature was not strongly dependent on incubation temperature. The results for the excipient emulsions were compared to curcumin-oil and curcumin-buffer mixtures that initially contained the same amount of curcumin. The particle size, microstructure, particle charge, and overall appearance of the different samples were then measured (FIGS. 21 to 23).

Particle size and system microstructure. The properties of the curcumin-emulsion mixture were evaluated at all stages of the GIT model, but the curcumin-oil mixture was only evaluated after incubation in the small intestine phase since reliable samples could not be obtained from the mouth or stomach phases. This was because the bulk oil tended to form a separate layer at the top of the mixtures, and therefore it was difficult to collect a representative sample. Similarly, the characteristics of the curcumin-buffer mixture were not determined in this series of experiments because it was difficult to collect reliable samples when there were only a few curcumin crystals present in a large volume of buffer solution.

In general, fairly similar trends were observed in the gastrointestinal behavior of curcumin-emulsion mixtures that had previously been incubated at either 30 or 100° C., and so the results are discussed together. The mean particle diameter (d₃₂) determined by static light scattering remained relatively constant after exposure to the mouth and stomach phases, but increased appreciably after exposure to the small intestine phase (FIG. 21a). Examination of the full particle size distributions of these emulsions indicated that a large fraction of the droplets had fairly similar sizes to the initial emulsions after exposure to the mouth and stomach phases, which suggested that they were relatively stable to coalescence, presumably because they had a non-ionic surfactant coating that was resistant to changes in pH, salt, and protease activity. Nevertheless, confocal microscopy images of the same samples indicated that extensive droplet flocculation occurred within the mouth and stomach phases (FIG. 23), which may have been due to depletion or bridging flocculation promoted by mucin originating from the artificial saliva. There appeared to be some dissociation of the flocs formed in the emulsions when they moved from the mouth to the stomach stage (FIG. 23). This behavior may be caused by a number of factors including sample dilution, changes in solution composition, and/or mechanical agitation.

The fact that the large particles observed in many of the samples by confocal microscopy were not observed by static light scattering suggests that the flocs dissociated upon dilution and stirring during sample preparation for the particle size analysis. This result highlights the importance of confirming light scattering measurements with microscopy measurements for this type of complex colloidal system.

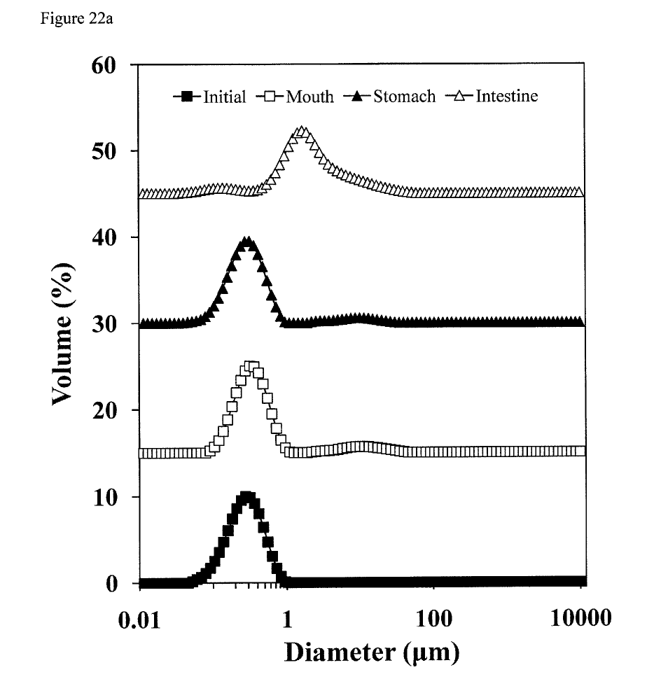

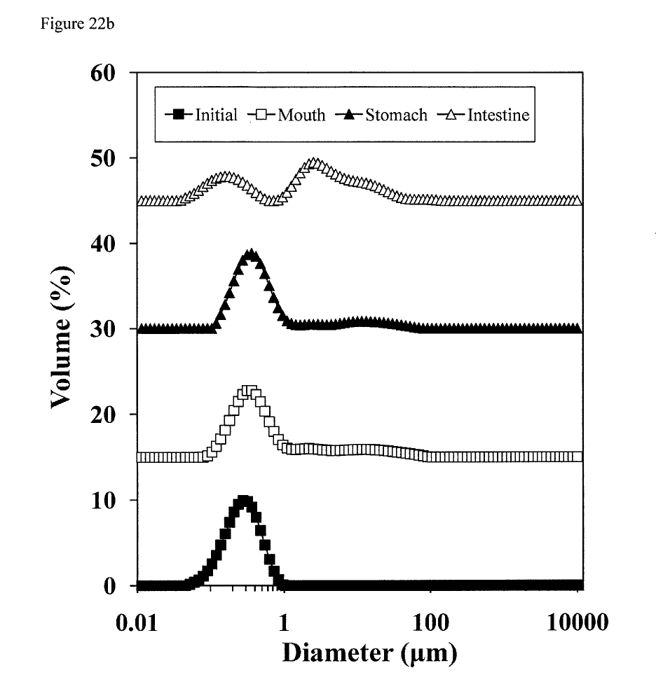

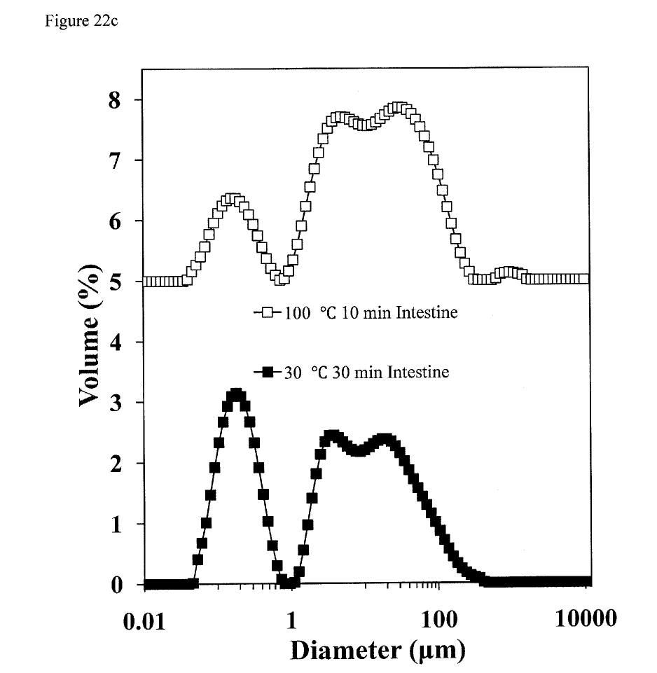

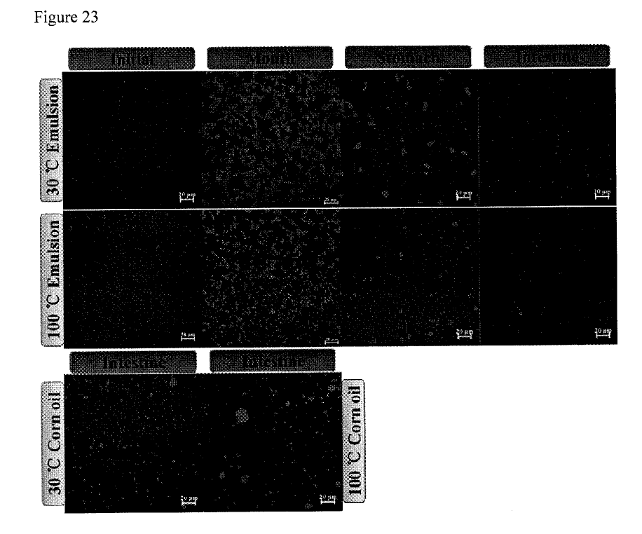

Light scattering measurements indicated that a population of relatively large particles was present in the curcumin-emulsion mixtures after exposure to the small intestine phase (FIGS. 22a and 22b), which was confirmed by confocal microcopy (FIG. 23). It is difficult to identify the precise nature of these particles because the intestinal digesta may contain various types of colloidal species, including non-digested lipids, micelles, vesicles, liquid crystals, and insoluble matter (such as calcium soaps).

Light scattering (FIG. 22) and confocal microscopy (FIG. 23) indicated that there were much larger particles present in the curcumin-oil mixtures after exposure to the small intestine phase than in the curcumin-emulsion mixtures. A possible explanation for this effect is that the bulk corn oil was digested more slowly than the pre-emulsified corn oil, and therefore there were some large non-digested lipid droplets present.

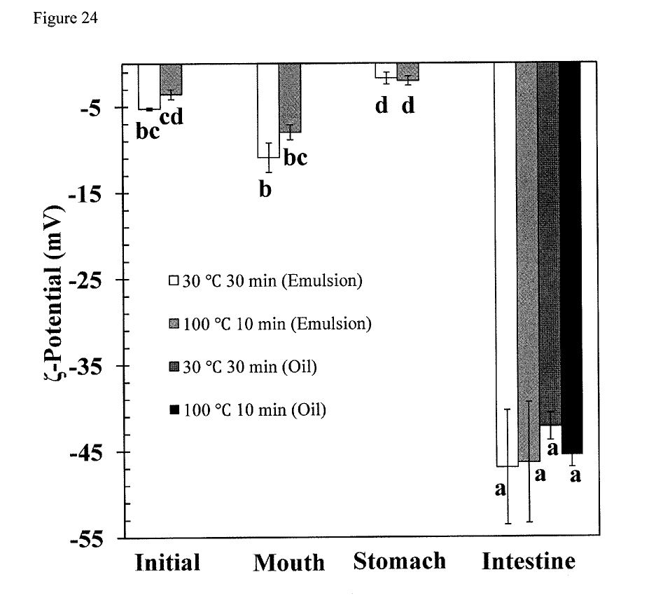

Electrical characteristics. The electrical characteristics (ζ-potential) of the particles in emulsion-curcumin mixtures exposed to different incubation temperatures followed similar trends after passage through each stage of the simulated GIT (FIG. 24), and so they will again be considered together. The particles in the initial curcumin-emulsion mixtures had relatively low negative charges (≈4 mV), which was due to the fact that a non-ionic surfactant was used to coat the droplets. The particles became appreciably more negative after exposure to the mouth phase (≈−9 mV), which may have been due to association of anionic species (such as mucin) with the lipid droplet surfaces. The particle charge became much less negative (≈−2 mV) after exposure to the stomach phase, which can be attributed to the relatively low pH and high ionic strength of the gastric fluids. Finally, the particle charge became highly negative (≈−47 mV) after exposure to the small intestinal fluids, which is probably due to the presence of various anionic constituents associated with this phase such as bile salts, phospholipids, and free fatty acids. There were no significant differences between the electrical characteristics of the particles in the small intestine phase for the curcumin-emulsion and curcumin-oil mixtures at either incubation temperature. This effect can be attributed to the fact that the electrical characteristics were dominated by the presence of bile salts, phospholipids, and free fatty acids, and were not strongly affected by sample microstructure.

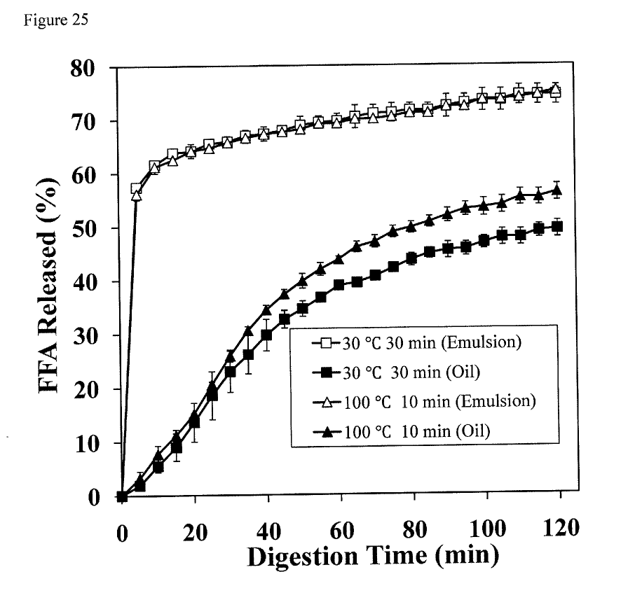

Lipid digestion. The pH stat method was used to determine the rate and extent of lipid digestion of curcumin-emulsion and curcumin-oil mixtures that had previously been incubated at either 30 or 100° C. The volume of NaOH that had to be titrated into the samples to maintain a constant pH (7.0) was measured as a function of digestion time, and then the fraction of free fatty released from the mixture was calculated (FIG. 25).

The initial incubation temperature (30 or 100° C.) had no effect on the rate and extent of lipid digestion for the curcumin-emulsion mixtures, which was probably due to the fact that the interfacial areas and compositions of the lipid droplets entering the small intestine phase were fairly similar (FIG. 25). In these systems, there was a rapid increase in FFAs during the first 10 minutes, followed by a more gradual increase at longer times. Conversely, there was a much less steep increase in the FFAs released over time in the curcumin-oil mixtures for both incubation times (FIG. 25). The origin of this effect can be attributed to the higher surface area of oil exposed to lipase for the emulsified corn oil than for the bulk corn oil. Indeed, the confocal microscopy images clearly highlighted that the fat droplets in the small intestine phase were much larger for the bulk oil than the emulsified oil (FIG. 23). Additionally, the rate and extent of lipid digestion in the curcumin-oil mixture that had been incubated at 100° C. (56% FFAs released after 2 hours) was higher than the one that had been incubated at 30° C. (49% FFAs released after 2 hours). The origin of this effect is unknown, but it may have been because the corn oil that had been incubated at the higher temperature was initially dispersed better in the water phase, i.e. formed smaller fat droplets (higher surface area).

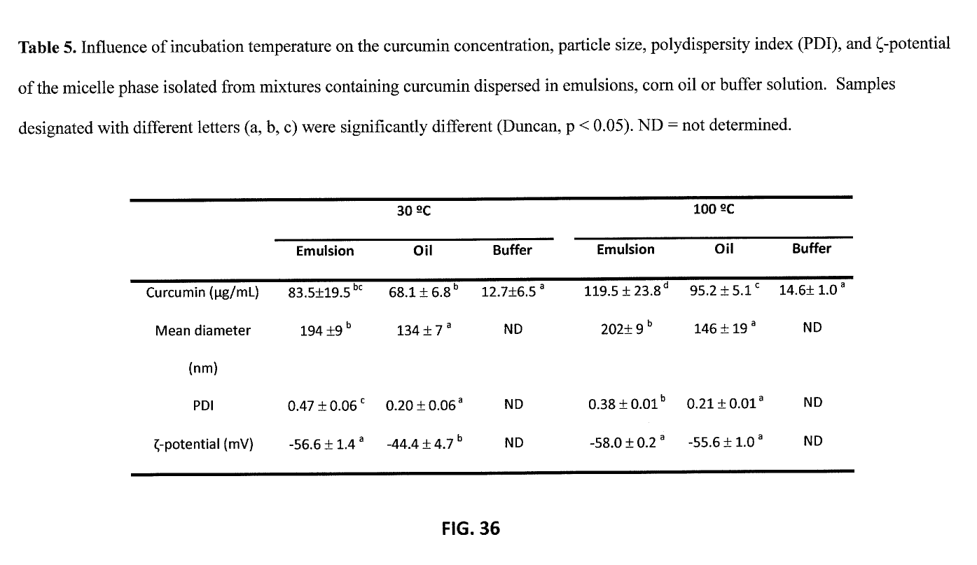

Curcumin solubilization and mixed micelle properties. The characteristics of the particles in the mixed micelle phase formed after exposure of the samples to simulated small intestine conditions were measured, as well as the amount of curcumin solubilized within the mixed micelle phase (Table 5) (FIG. 36). The mixed micelle phase was collected by centrifugation, so that any large particles observed in the small intestine digesta should have been removed. All of the mixed micelle samples contained highly negatively charged particles, which can be attributed to the fact that they consisted primarily of bile salts, phospholipids, and free fatty acids. The mean particle diameters in the micelle phase were around 100 to 200 nm, which suggests that they were probably vesicles since true micelles are much smaller than this (<10 nm). Surprisingly, the particles in the mixed micelle phase collected from digestion of the bulk oils were appreciably smaller than those collected from digestion of the emulsified oils (Table 5). There are a number of potential mechanisms that could account for this observation, such as differences in the rate and extent of lipid digestion, and the presence of non-ionic surfactant in the emulsions. For both the curcumin-emulsion and curcumin-oil systems, there was no major difference between the size of the particles in samples that had been incubated at 30 or 100° C.

The amount of curcumin present in the mixed micelle phase is a good indication of its bioaccessibility, i.e., the amount available for absorption. Overall, the concentration of curcumin measured in the mixed micelle phase was higher for the digested curcumin-emulsion mixtures than for the digested curcumin-oil mixtures (Table 5). This suggests that there was more efficient transfer of the curcumin into the mixed micelles for the emulsion than for the bulk oil. There are two major reasons for this phenomenon: (i) some of the bulk oil was not digested, and so some of the curcumin may have remained dissolved within this oil phase; (ii) more of the emulsified oil was digested, and so there will have been more mixed micelles available to solubilize the curcumin. The curcumin concentration in the mixed micelles obtained from digestion of the curcumin-emulsion mixture incubated at 100° C. was appreciably higher than that for the mixture incubated at 30° C. This effect was probably due to the fact that a higher fraction of the crystalline curcumin was solubilized within the emulsion held at the higher incubation temperature. The amount of curcumin present within the mixed micelle phase resulting from digestion of the curcumin-oil mixture was also higher for the sample incubated at 100° C. than for the one incubated at 30° C. This effect may again be due to the fact that a higher amount of curcumin was solubilized in the oil phase (rather than present as crystals) prior to digestion. In addition, there was a greater extent of lipid digestion in the bulk oil incubated at 100° C., which may have resulted in greater curcumin release and solubilization.

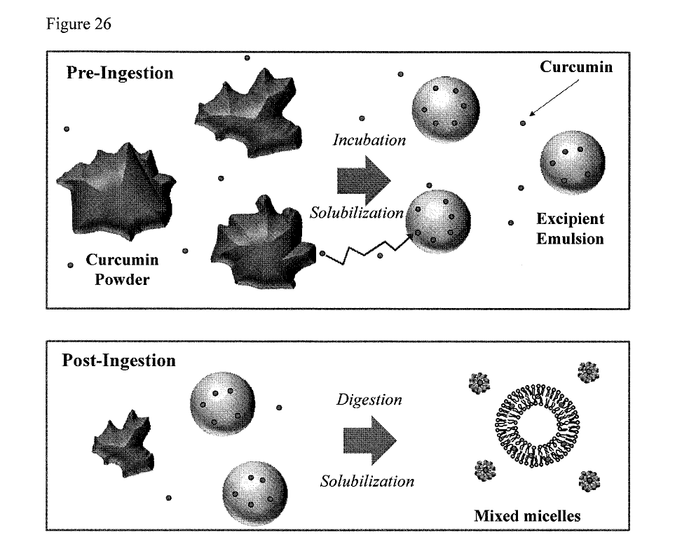

Potential Mechanisms. While not intending to be held to a particular theory, the potential mechanisms responsible for the increase in bioaccessibility of curcumin by excipient emulsions are highlighted in FIG. 26. Prior to ingestion, curcumin may be solubilized within the oil droplets when powdered curcumin is incubated with the emulsions. This may occur due to diffusion of curcumin molecules through the aqueous phase, and occurs more rapidly for emulsifier oil than for bulk oil due to the higher surface area and shorter diffusion pathway. In addition, this process occurs more rapidly at higher temperatures due to the higher solubility of curcumin in the oil and water phases. After ingestion, curcumin may be solubilized within the mixed micelles resulting from digestion of the oil droplets. The transfer of curcumin into the mixed micelles may be more efficient for emulsified oil than for bulk oil due to the faster rate and greater extent of lipid digestion. Consequently, there are more mixed micelles available for the curcumin to the solubilized within.

In summary, the present teachings have shown that excipient emulsions can be used to increase the bioaccessibility of powdered curcumin. A greater amount of curcumin is transferred from the powder into the lipid droplets for curcumin-emulsion mixtures incubated at 100° C. than for those incubated at 30° C. This effect was attributed to the fact that solubility of curcumin in the water and oil phases increases with increasing temperature, as well as the mass transport rate. It was shown that the curcumin concentration in the mixed micelle phase formed after exposure to a simulated gastrointestinal tract depended on the nature of the food matrix, decreasing in the following order: emulsified oil>bulk oil>buffer solution. This effect was attributed to the increased solubilization capacity of the small intestinal fluids when a triglyceride oil is broken down into free fatty acids and monoglycerides that are incorporated into mixed micelles. In addition, the curcumin concentration in the mixed micelle phase was higher for curcumin-emulsion or curcumin-oil mixtures that had been incubated at 100° C. than for those that had been incubated at 30° C., which was attributed to a greater solubilization of the curcumin into the oil phase prior to digestion.

### Example 4

### Influence of Lipid Droplet Size on Solubility and Bioaccessibility of Powdered Curcumin

Example 3 above shows that excipient emulsions can increase the solubility and bioaccessibility of powdered curcumin. Curcumin molecules were transferred from the curcumin powder into the oil droplets in the emulsion during incubation at elevated temperatures, which increased the subsequent bioaccessibility of the curcumin when the emulsion was passed through a simulated gastrointestinal tract (GIT). In the present example, the influence of the size of the oil droplets in the excipient emulsions on the bioaccessibility of powdered curcumin was examined. Examples 1 and 2 above show that decreasing droplet size increases the bioaccessibility of highly lipophilic bioactive molecules (such as β-carotene and Coenzyme Q10) solubilized within emulsions. This effect was attributed to the influence of droplet size on the efficiency of lipid phase digestion, bioactive release, and intestinal fluid solubilization capacity.

In the present example, excipient emulsions with three different particle sizes (small, medium, and large) were produced using a high shear mixer and/or a microfluidizer. These excipient emulsions were mixed with powdered curcumin, and then incubated at two different temperatures (30° C. for 30 min or 100° C. for 10 min) to mimic thermal conditions that might be encountered in actual food applications: (i) mixing curcumin powder with an excipient salad dressing at ambient temperature; (ii) mixing curcumin powder with an excipient cooking sauce at elevated temperatures (e.g., curry sauce). The effect of droplet size and incubation conditions on the transfer of curcumin into the excipient emulsions prior to ingestion was measured. In addition, the influence of droplet size on the bioaccessibility of the curcumin after exposure of the curcumin-excipient emulsions to simulated GIT conditions was measured.

Materials: Corn oil purchased from a local supermarket was used as an example of a digestible long chain triglyceride. The following chemicals were purchased from the Sigma Chemical Company (St. Louis, Mo., USA): curcumin (SLBH2403V); mucin from porcine stomach (SLBH9969V); pepsin from porcine gastric mucosa (SLBL1993V); lipase from porcine pancreas pancreatin (SLBH6427V); porcine bile extract (SLBK9078); Tween 80 (BCBG4438V); and Nile Red (063K3730V). The composition of the curcumin was determined by HPLC as curcumin (77.9%), demethoxycurcumin (20.2%), and bisdemethoxycurcumin (1.9%) using a method described earlier Zheng, J., et al., “Identification of novel bioactive metabolites of 5-demethylnobiletin in mice”, Molecular Nutrition & Food Research, 57(11):1999-2007, (2013), which is incorporated herein in its entirety. All other chemicals were of analytical grade. Double distilled water was used to prepare all solutions and emulsions.

For curcumin and total curcuminoid content determination a HPLC method was developed based on one described previously in Zheng, J., et al. “Identification of novel bioactive metabolites of 5-demethylnobiletin in mice”, Molecular Nutrition & Food Research, 57(11):1999-2007, (2013), which is incorporated herein in its entirety. Briefly, an integrated HPLC system (Agilent 1100 series, Agilent Technologies, Santa Clara, Calif., USA) equipped with a binary solvent delivery system, an on-line degasser, an auto-sampler, a column temperature controller, a diode array detector (DAD), and a variable wavelength detector (VWD) was used to quantify curcumin. System control and data analysis were processed using instrument software (Agilent ChemStation). A RP-Amide HPLC column (15 cm×4.6 mm id, 3 μm, Ascentis Express, Sigma Aldrich Corp., St. Louis, Mo., USA) was used as the stationary phase. The flow rate and injection volume were 1 mL/min and 50 μL, respectively. The two mobile phases consisted of: Mobile phase A, deionized water; Mobile phase B, acetonitrile. The linear solvent gradient consisted of 10% B at the beginning, 5% B at 20.0 min, 5% B at 20.0 min, 100% B at 90.0 min, 100% B at 93.0 min, 5% B at 95.0 min, and 5% B at 100.0 min. The detection wavelength was set at 419 nm.

Excipient Emulsion Preparation

Initially, an aqueous phase was prepared by mixing 1% (w/w) Tween 80 with 99% (w/w) aqueous buffer solution (10.0 mM phosphate buffer saline (PBS), pH 6.5) and stirring for at least 2 h. Excipient emulsions with different particle size distributions were prepared as follows. A “large” emulsion was prepared by mixing 10% (w/w) corn oil with 90% (w/w) Tween 80 aqueous phase using a high-shear mixer for 2 min (M133/1281-0, Biospec Products, Inc., ESGC, Basle, Switzerland). “Small” and “medium” emulsions were prepared by passing the large emulsion three times through a microfluidizer (M110Y, Microfluidics, Newton, Mass., USA) with a 75 μm interaction chamber (F20Y) at an operating pressure of 12,000 or 6,000 psi, respectively.

Preparation of Curcumin-Emulsion Mixtures

Curcumin (9 mg) was weighed into a beaker and then excipient emulsion (30 mL) was added. This amount of emulsion was utilized since it had the ability to completely dissolve the curcumin based on the measured equilibrium solubility of curcumin reported in corn oil at ambient temperature≈3.2 f 0.1 mg/mL (Ahmed et al., 2012). The resulting mixtures were then incubated at either 30° C. for 30 min or 100° C. for 10 min to simulate a salad dressing or a cooking sauce, respectively. After incubation, selected samples were immediately placed in an ice water and then used for the following experiments.

Curcumin Solubility in Mixtures

The solubility of curcumin in each mixture was measured spectrophotometrically using a method based on that reported by Ahmed, K., Li, Y., McClements, D. J., and Xiao, H., “Nanoemulsion- and emulsion-based delivery systems for curcumin: Encapsulation and release properties”, Food Chemistry, 132(2):799-807 (2012), which is incorporated herein in its entirety, with some modifications. 10 mL of mixture was collected, and then centrifuged at 1750 rpm (≈940×G) for 10 min at ambient temperature (CL10 centrifuge, Thermo, Scientific, Pittsburgh, Pa., USA) to remove any non-dissolved (crystalline) curcumin. One millilitre of supernatant was then mixed with 5 mL chloroform, vortexed, and centrifuged at 1750 rpm (≈940×G) for 10 min at ambient temperature. The bottom layer containing the solubilized curcumin was collected, while the top layer was mixed with an additional 5 mL of chloroform and the same procedure was repeated. The two bottom chloroform layers were combined, and diluted to a concentration suitable to be analysed by a UV-VIS spectrophotometer at 419 nm (Ultraspec 3000 pro, GE Health Sciences, Pittsburgh, Pa., USA). A cuvette containing pure chloroform was used as a reference cell. The concentration of curcumin extracted from each mixture was calculated from a calibration curve of absorbance versus curcumin concentration in chloroform. The solubility of curcumin in each mixture was then calculated as the concentration of curcumin extracted from each mixture multiplied by the dilution factor.

Particle Characterization

The mean particle diameter and particle size distribution of both curcumin-emulsion mixtures after incubation at different temperatures and mixtures exposed to simulated gastrointestinal conditions were monitored using static light scattering (Mastersizer 2000, Malvern Instruments Ltd., Malvern, Worcestershire, UK). Samples were diluted with appropriate buffer solutions and stirred in the dispersion unit at a speed of 1200 rpm. The particle size is reported as the surface-weighted (d₃₂) mean diameter. The electrical charge (ζ-potential) of the particles in the samples was measured using a micro-electrophoresis instrument (Nano-ZS, Malvern Instruments, Worcestershire, UK). Samples were diluted with appropriate buffer solutions prior to measurements to avoid multiple scattering.

The mean particle diameters (Z-average), particle size distributions, and electrical charges (ζ-potential) of micelles collected by centrifugation of the raw digesta were measured by dynamic light scattering and micro-electrophoresis (Nano-ZS, Malvern Instruments, Worcestershire, UK). Micelles were diluted with buffer solution (5 mM PBS, pH 7.0) prior to measurements to avoid multiple scattering effects.

Microstructural Analysis

The microstructure of samples was characterized using confocal scanning fluorescence microscopy (Nikon D-Eclipse C1 80i, Nikon, Melville, N.Y., USA). The samples analysed by confocal microscopy were dyed with an oil-soluble fluorescent dye (0.1% Nile Red) to highlight the location of the lipid phases. All images were captured with a 10× eyepiece and a 60× objective lens (oil immersion) to give a total magnification of 600×. The presence of crystalline curcumin in the mixtures was determined using a cross-polarized lens (C1 Digital Eclipse, Nikon, Tokyo, Japan).

Simulated Gastrointestinal Digestion

Powdered curcumin was mixed with excipient emulsion, and then held at 100° C. for 10 min. Each sample was passed through a three-step GIT model that consisted of mouth, stomach, and small intestine phases.

Initial system: The curcumin-excipient emulsion mixtures were placed into a glass beaker in a shaking incubator (Innova Incubator Shaker, Model 4080, New Brunswick Scientific, New Brunswick, N.J., USA) at 37° C. The initial concentrations of curcumin, corn oil, and emulsifier was the same in all curcumin-excipient emulsion mixtures, only the lipid droplet size was different.

Mouth phase: A simulated saliva fluid (SSF) containing 3 mg/mL mucin and various salts was prepared as described previously (Mao & McClements, 2012). SSF was preheated to 37° C. and then mixed with the preheated curcumin-excipient emulsion mixture at a 1:1 mass ratio. The mixture was then adjusted to pH 6.8 and placed in a shaking incubator at 100 rpm and 37° C. for 10 min to mimic oral conditions. In reality, a food would not spend this length of time within the mouth, but these conditions were used to standardize the testing procedure and reduce batch-to-batch variations.

Stomach phase: Simulated gastric fluid (SGF) was prepared by placing 2 g NaCl and 7 mL HCl into a container, and then adding double distilled water to 1 L. The bolus sample from the mouth phase was then mixed with SGF containing 0.0032 g/mL pepsin preheated to 37° C. at a 1:1 mass ratio. The mixture was then adjusted to pH 2.5 and placed in a shaker at 100 rpm and 37° C. for 2 hours to mimic stomach digestion. Gastric lipase was not included in the simulated gastric fluids due to the difficulty in obtaining reliable and inexpensive samples. Nevertheless, in reality gastric lipase does promote some lipid digestion within the stomach, which may have some impact on the subsequent behaviour of ingested emulsions.

Small Intestine phase: 24 mL chyme samples from the stomach phase were diluted with buffer solution (10 mM PBS, 6.5) to obtain a final corn oil concentration of 1.0%. The diluted chyme was then incubated in a water bath (37° C.) for 10 min, and then the solution was adjusted back to pH 7.0. Next, 3 mL of simulated intestinal fluid (containing 0.5 M CaCl₂ and 7.5 M NaCl) were added to 60 mL digesta. Then, 7 mL bile extract, containing 375.0 mg bile extract (pH 7.0, PBS), was added with stirring and the pH was adjusted to 7.0. Finally, 5 mL of pancreatic suspension, containing 120 mg of lipase (pH 7.0, PBS), was added to the sample and an automatic titration unit (Metrohm, Inc., Riverview, Fla., USA) was used to monitor the pH and control it to a fixed value (pH 7.0) by titrating 0.25 M NaOH solution into the reaction vessel for 2 h at 37° C. The percentage of free fatty acids released in the sample was calculated from the number of moles of NaOH required to maintain neutral pH as described previously (Li & McClements, 2010).

Determination of Curcumin Concentration in Simulated Intestinal Fluids

After in vitro digestion, 30 mL raw digesta of each mixture were centrifuged (18,000 rpm, ≈38,465×G, Thermo Scientific, Waltham, Mass., USA) at 25° C. for 30 min. The clear supernatant was collected and assumed to be the “micelle” fraction in which the curcumin was solubilized. In some samples, a layer of non-digested oil was observed at the top of the samples and it was removed from the micelle fraction. Aliquots of 5 mL of raw digesta or micelle fraction were mixed with 5 mL of chloroform, vortexed, and then centrifuged at 1750 rpm (≈940×G) for 10 min at ambient temperature. The bottom layer containing the solubilized curcumin was collected, while the top layer was mixed with an additional 5 mL of chloroform and the same procedure was repeated. The two collected chloroform layers were combined together, and then diluted to an appropriate concentration to be analysed by a UV-visible spectrophotometer at 419 nm. The concentration of curcumin in the raw digesta and micelle phase were then calculated from the measured absorbance using a standard curve, taking into account the dilution factor. Curcumin bioaccessibility was calculated using the following equation:

Bioaccessibility=100×(C_{Micelle}/C_{Raw Digesta}) (3)

where, C_{Micelle} and C_{Raw Digesta} are the concentrations of curcumin in the micelle fraction and in the raw digesta after the pH-stat experiment, respectively.

The UV-visible spectrophotometer method used herein only gives a crude indication of the overall concentration of curcumin present in the system. This method does not provide information about the behaviour of the different forms of curcumin: curcumin, demethoxycurcumin and bis-demethoxycurcumin. However, this simple method is suitable for the purposes herein since the main focus was to establish the potential impact of excipient emulsion characteristics on curcumin bioavailability.

Statistical Analysis

All experiments were carried out on at least two freshly prepared samples. The results are expressed as means±standard deviations (SD). Data was subjected to statistical analysis using SPSS software version 18.0, and differences were considered significant at p<0.05.

Results and Discussion

Influence of Initial Droplet Size and Thermal Incubation on Properties of Curcumin-Excipient Emulsion Mixtures

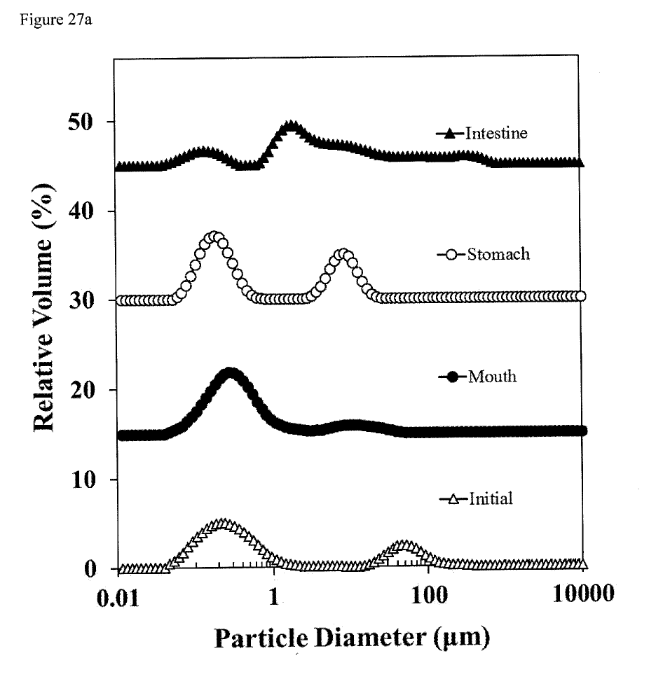

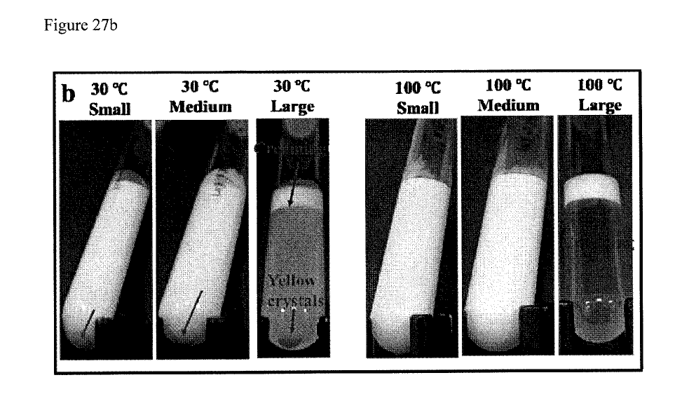

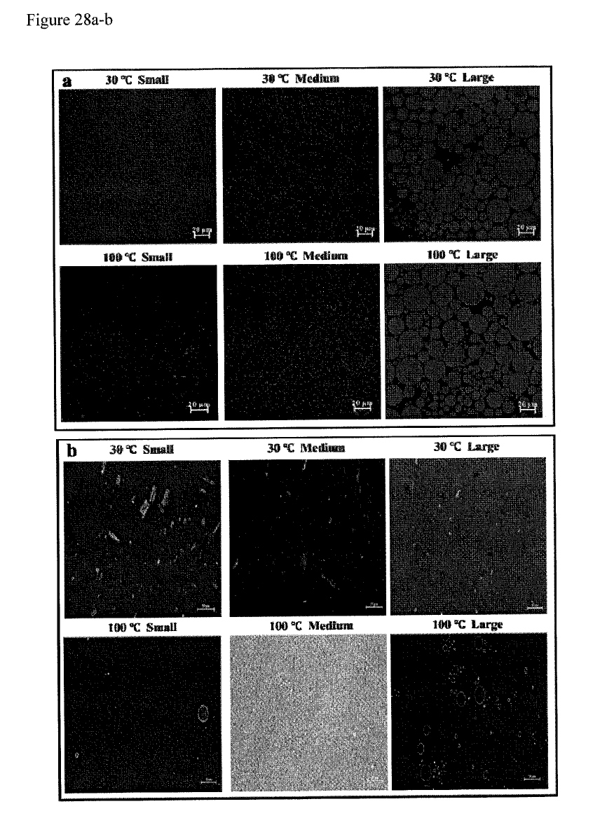

Excipient emulsions with different initial mean droplet diameters (d₃₂=0.18, 0.52 and 13.6 μm, for small, medium, and large emulsions) were mixed with powdered curcumin, and then exposed to two different incubation temperatures to simulate conditions they might experience in food applications: (i) 30° C. to represent salad dressings at ambient temperature; (ii) 100° C. to represent sauces at cooking temperatures. The mixture of curcumin and excipient emulsion was stirred to ensure homogeneity, and then incubated at either 30° C. for 30 min or at 100° C. for 10 min. The curcumin-excipient emulsion mixtures did not show any appreciable changes in droplet characteristics after incubation at 30° C. for 30 min. Indeed, there were no significant changes in mean particle diameters or particle size distributions when compared to the initial excipient emulsions (FIG. 27a). There was little change in the droplet characteristics of the curcumin-excipient emulsion mixtures containing medium or large droplets after incubation at 100° C. for 10 min. However, there was a slight increase in the mean droplet diameters (d₃₂=0.24, 0.57 and 15.7 am) after heating at 100° C. for 10 min. In addition, there was evidence of a population of large particles in the particle size distribution of the excipient emulsions initially containing the smallest droplets (FIG. 27b). These results suggest that there was some aggregation of the droplets in this emulsion during incubation at high temperatures. It is possible that the relatively high collision frequency of the droplets in the small emulsion led to some coalescence.

There was no significant change in the electrical charge on the droplets in any of the excipient emulsions after incubation at either 30° C. (ζ≈−5 mV) or 100° C. (ζ≈−5 mV), which suggests that the interfacial composition remained relatively constant.

The microstructures of the curcumin-excipient emulsions were recorded after exposure to the two thermal treatments using confocal fluorescence microscopy (FIG. 28a). There was no appreciable change in the microstructures of the curcumin-excipient emulsion mixtures for all three droplet sizes after incubation at 30° C. for 30 min. The microstructures of the initial mixtures is not shown because it was similar to that of the mixtures held at ambient temperature. There was also no observable change in the microstructures of the curcumin-excipient emulsion mixtures initially containing medium or large droplets after incubation at 100° C. for 10 min. However, there was evidence of some larger oil droplets in the mixed systems that initially contained small droplets after incubation at 100° C., suggesting some coalescence occurred, which was in agreement with the particle size measurements (FIG. 27b). A thin layer of oil was visible at the top of the large excipient emulsions, which was not detected in the light scattering or confocal microscopy results. This layer was an orangey-yellow colour, which suggested that at least some of the curcumin was solubilized within it.

The effect of particle size on the amount of curcumin transferred into the excipient emulsions was also measured after incubation at 30° C. for 30 min or at 100° C. for 10 min. The amount of curcumin transferred into the excipient emulsions depended on incubation temperature and droplet size. For example, the amount of curcumin present within the small, medium, and large emulsions was around 66±1, 104±6, and 88±26 μg/mL at 30° C., while it was around 280±38, 270±8, and 230±27 g/mL at 100° C., respectively. The amount of curcumin present within the excipient emulsions was significantly higher (p<0.05) after incubation at 100° C. than at 30° C., and was significantly higher (p<0.05) for the medium and large droplets than for the small droplets at 30° C. A number of physicochemical factors may have affected the amount of curcumin detected in the excipient emulsions. First, the rate of uptake of curcumin by the emulsions could increase with decreasing droplet size because of their higher surface area. Second, the rate of chemical degradation of curcumin could increase with decreasing droplet size, since then more of the curcumin molecules would be exposed to the aqueous phase. These competing effects may account for the fact that, surprisingly, the highest amount of curcumin was detected in the medium emulsions incubated at 30° C.

After incubation at 100° C., the amount of curcumin solubilized in the excipient emulsions containing small and medium droplets (270-280 μg/mL) was appreciably higher than that those containing the large droplets (230 μg/mL). This may have been partly due to the fact that some droplet coalescence and oiling off occurred in the large emulsions held at the higher temperature, and some of the curcumin therefore remained in the upper oil phase and was not therefore measured. In addition, droplet size may have influenced the rate of transfer of curcumin into the droplets, as well as the rate of chemical degradation of curcumin.

The presence of curcumin crystals within the curcumin-excipient emulsion mixtures after incubation at 30 or 100° C. was observed using a crossed polarizer lens (FIG. 28b). Crystalline material was clearly observed in all the mixtures incubated at 30° C., which suggested that the curcumin crystals did not completely dissolve when held at this temperature in the presence of the excipient emulsions. Conversely, no crystals were observed in any of the mixtures held at 100° C., which indicated that curcumin crystals rapidly and completely dissolved within all the excipient emulsions at this elevated temperature. However, there was evidence of some large droplets in both small and large emulsions after heating at 100° C., which again suggested that some droplet coalescence had occurred.

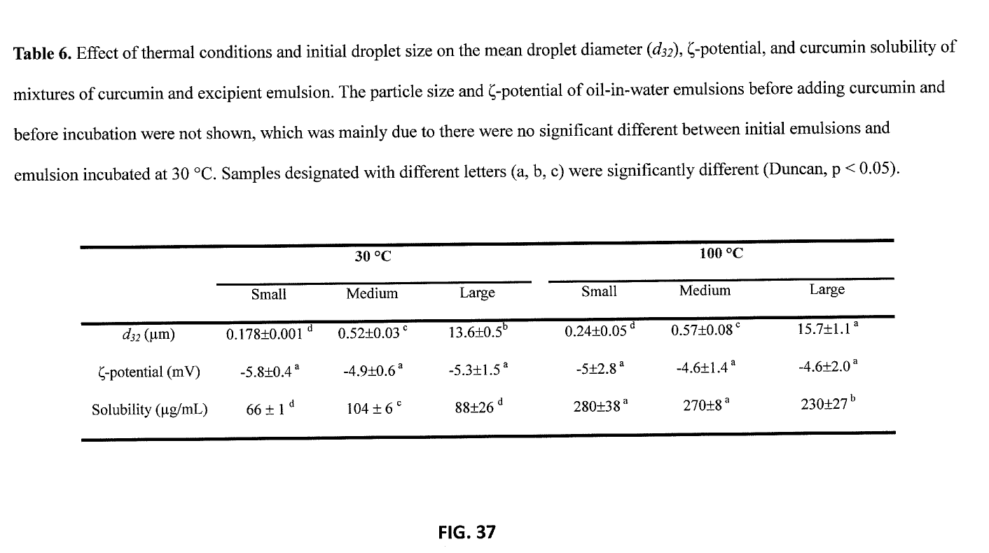

See also, Table 6 (FIG. 37) for the effect of thermal conditions and initial droplet size on various parameters.

Influence of Initial Droplet Size on Gastrointestinal Behaviour of Curcumin—Excipient Emulsions

The influence of the initial droplet size of excipient emulsions on their potential biological fate was examined using an in vitro gastrointestinal tract (GIT) model that simulates the mouth, stomach, and small intestine phases. Only the curcumin-excipient emulsions incubated at 100° C. for 10 min were selected for the GIT study because a greater amount of curcumin was solubilized within them.

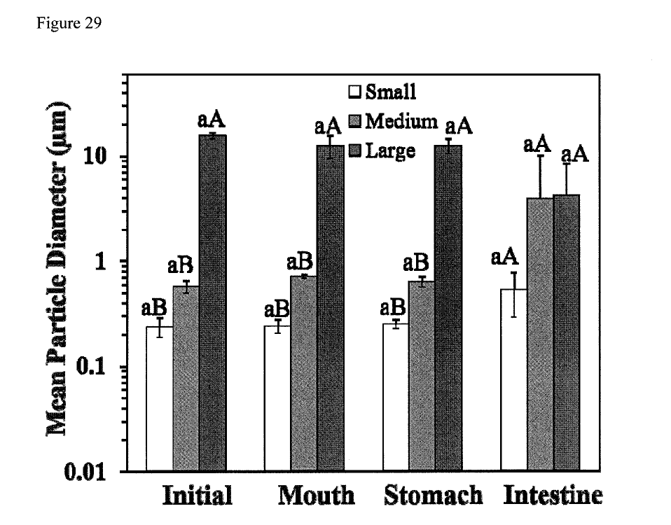

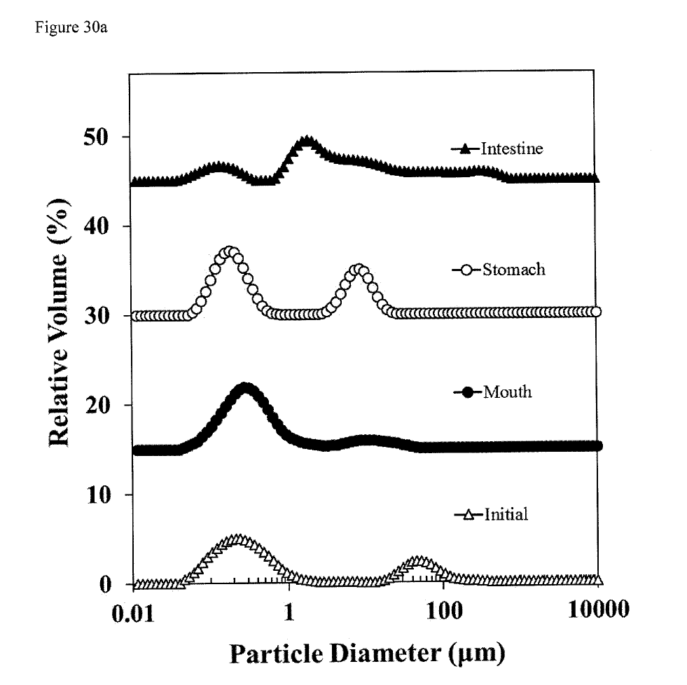

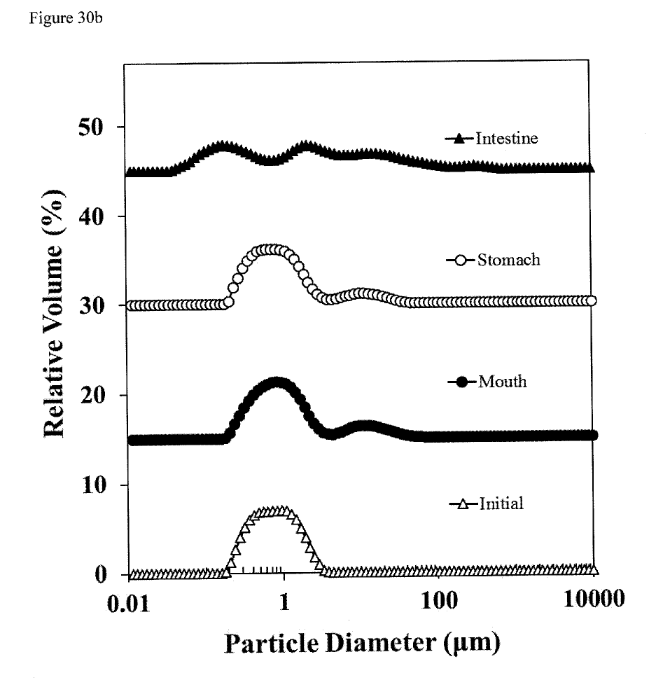

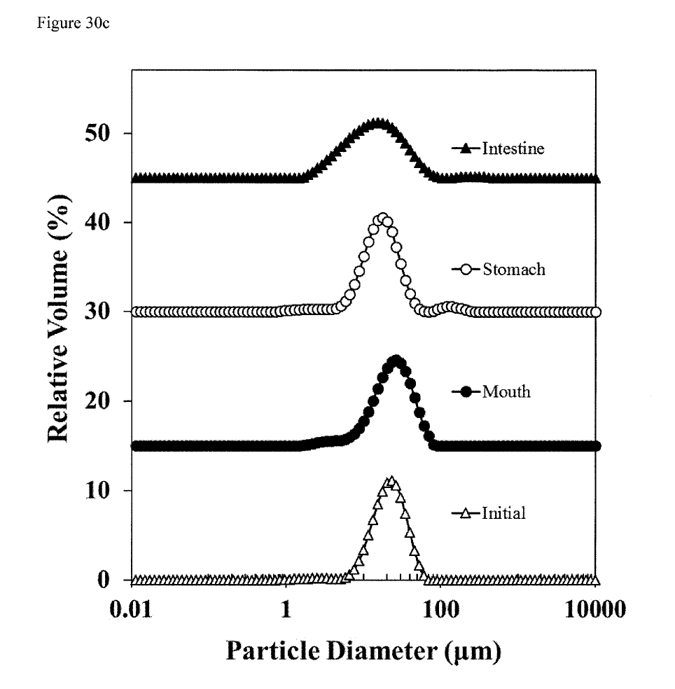

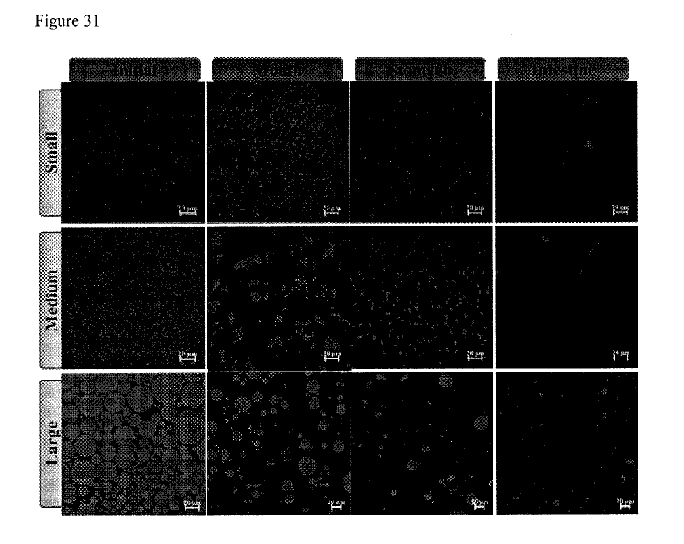

Particle size and microstructure. The properties of the curcumin-excipient emulsion mixtures were evaluated at each stage of the GIT model. Initially, the mean particle diameter (d₃₂) determined by static light scattering of small, medium, and large excipient emulsions after incubation at 100° C. for 10 min was 0.24, 0.57 and 16 μm, respectively (FIG. 29). In addition, the initial emulsions all had monomodal particle size distributions (FIG. 30), which suggested that the homogenization methods used were efficient. The light scattering results are in agreement with the microstructural images of the initial emulsions obtained by confocal fluorescence microscopy (FIG. 31).

For all the excipient emulsions, the mean particle diameter remained relatively constant and the particle size distribution remained monomodal after exposure to the mouth and stomach phases (FIGS. 29 and 30), which suggests that the droplets were relatively stable to coalescence under these conditions. This effect can be attributed to the fact that the droplets were coated by a non-ionic surfactant (Tween 80) that primarily stabilizes the droplets due to the steric repulsion generated by the hydrophilic polymeric head groups. Consequently, changes in pH, ionic strength, and protease activity did not have a strong influence on their coalescence stability. Despite the fact that the static light scattering technique indicated that there was no increase in the mean particle diameter, the confocal microscopy images clearly indicated that extensive droplet flocculation occurred in the mouth and stomach, particularly for the excipient emulsions initially containing small- and medium-sized droplets (FIG. 31). This suggests that the droplets were held together by relatively weak attractive forces in the flocs, which were disrupted when the samples were diluted for the particle size analysis.

There was an appreciable change in the particle size distribution and confocal microscopy images of all the excipient emulsions when they moved from the stomach to the small intestine (FIGS. 30 and 31). These microstructural changes can be attributed to a number of different physicochemical events occurring within the simulated small intestine phase: (i) digestion of lipid droplets by lipase; (ii) aggregation of lipid droplets; (iii) formation of mixed micelles, vesicles, and other colloidal structures; and, (iv) formation of insoluble matter (such as calcium soaps).

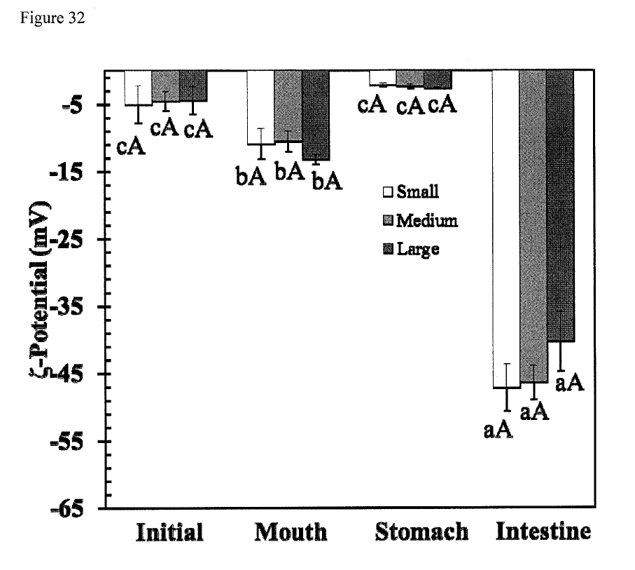

Electrical characteristics. The electrical characteristics (ζ-potential) of the particles in the curcumin-excipient emulsion mixtures after exposure to the different regions of the simulated GIT were also measured (FIG. 32). The mixtures formed from all three initial excipient emulsions (small, medium, and large) behaved fairly similarly and so the results will be considered together. The particles in the initial mixtures had relatively low negative charges (≈−5 mV), which is due to the fact that a non-ionic surfactant was used to coat the droplets. After exposure to the mouth phase the particles in the mixtures became more highly negative (≈−10 mV), which may have been due to association of anionic species (such as mucin) with the lipid droplet surfaces (Salvia-Trujillo et al., 2013). After exposure to the stomach phase, the particle charge became considerably less negative (≈−2 mV), which can be attributed to the relatively low pH and high ionic strength of the gastric fluids. The low pH will lead to protonation of any free fatty acids (—COOH) present thereby reducing droplet net charge, whereas the high ionic strength will lead to electrostatic screening of any surface charges. After exposure to the small intestinal fluids, the particle charge became highly negative (≈−40 to −50 mV), which can be attributed to the presence of various anionic constituents in this phase such as bile salts, phospholipids, and free fatty acids. There were no significant differences between the electrical characteristics of excipient emulsions with different particle sizes after exposure to each stage of the GIT model (FIG. 32). This effect suggests that the interfacial composition of the lipid droplets in the excipient emulsions was not strongly dependent on their initial particle size.

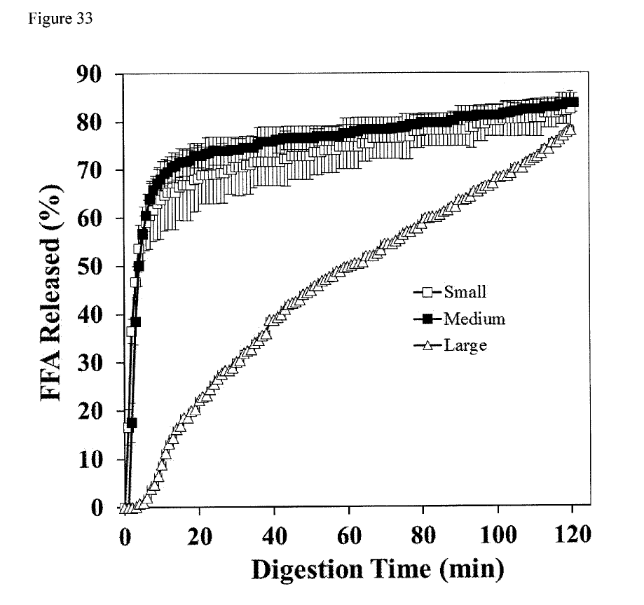

Lipid digestion. The pH stat method was used to evaluate the effect of initial droplet size of the excipient emulsions on the rate and extent of lipid digestion. A curcumin-excipient emulsion mixture was prepared by incubating the two components together at 100° C. for 10 min to simulate the preparation of a cooking sauce. The volume of NaOH that had to be titrated into the samples to maintain a constant pH (7.0) was then measured as a function of digestion time, and then the fraction of free fatty acids released from the mixture was calculated. The initial particle size of the excipient emulsion had a significant effect on the rate and extent of lipid digestion (FIG. 33). In the case of the small and medium excipient emulsions, there was a rapid increase in FFAs during the first 10 minutes, followed by a more gradual increase at longer times, until a relatively constant final value was attained. For the large excipient emulsions, there was a much slower initial release of FFAs. Nevertheless, the final amount of FFAs released after 2 h intestinal digestion was relatively high and fairly similar for all three emulsions, which suggested that the majority of triacylglycerols had been fully digested.

To a first approximation, the percentage of free fatty acids released (Φ) from an oil-in-water emulsion as a function of digestion time (t) can be modelled by the following equation:

Φ=φₘₐₓ(1−(1+3kMₜ/2d₀ρ₀)⁻²) (4)

Here, φₘₐₓ is a measure of the total extent of lipid digestion (i.e., the percentage of FFAs released by the end of digestion), k is the normalized digestion rate (i.e., μmols of FFA released per unit droplet surface area per unit time), d₀ is the initial mean droplet diameter (d₃₂), ρ₀ is the oil droplet density (≈910 kg m⁻³ for corn oil), and M is the molar mass of the oil (≈0.875 kg mol⁻¹ for corn oil). A pH-stat digestion profile can then be characterized in terms of φₘₐₓ and k, which can be determined by finding the values that give the best fit between the experimental data and the mathematical model. This equation can also be used to calculate the “digestion time”, which is the time (t_{D}) required for the FFAs released to increase to 50% of φₘₐₓ as shown previously by Li, Y. and McClements, D. J., “New mathematical model for interpreting pH-Stat digestion profiles: Impact of lipid droplet characteristics on in vitro digestibility”, Food Chemistry, 58(13):8085-92 (2010) and Salvia-Trujillo, et al., “Influence of particle size on lipid digestion and β-carotene bioaccessibility in emulsions and nanoemulsions”, Food Chemistry, 141(2):1472-80 (2013), both of which are incorporated herein in their entirety. The parameters determined for the three different excipient emulsions by fitting this equation to the initial part of the curve (to obtain k) and to the latter part of the curve (to obtain φₘₐₓ) are summarized here: φₘₐₓ=82.2, 83.7, and 77.5; k=0.5, 1.1, and 1.4 μmol s⁻¹ m⁻²; t_{D}=2.4, 2.8 and 43 minutes for small, medium and large droplets, respectively. The mean particle diameters used in the calculations were those of the emulsions after passage through the stomach phase: small (0.25 μm), medium (0.64 μm), and large (12.5 μm).

The mathematical analysis of the digestion curves provides some important information about the influence of initial droplet size on the rate of droplet digestion. The normalized digestion rate (k) actually decreased with decreasing droplet size, which means that the amount of FFAs produced per unit surface area per unit time decreased, which was possibly due to the fact that the total amount of lipase present was the same in all of the emulsions, and therefore the amount of lipase per unit surface area decreased as the droplet size decreased.

Curcumin Bioaccessibility and Mixed Micelle Properties

Curcumin has numerous biological and pharmacological activities that may be beneficial to human health. However, its low oral bioavailability may limit its effectiveness as a nutraceutical agent in foods. Two physicochemical mechanisms may limit the bioavailability of curcumin: low gastrointestinal solubility and poor chemical stability. Curcumin is a highly lipophilic molecule that has a low water-solubility, and therefore it must be solubilized within mixed micelles prior to uptake by the epithelium cells. Curcumin is also highly unstable to chemical degradation around neutral pH and above, which results in the formation of various reaction products, such as trans-6-(4′-hydroxy-3′-methoxyphenyl)-2,4-dioxo-5-hexanal, ferulic acid, feruloylmethane, and vanillin. Therefore the curcumin concentrations in the total raw digesta and in the mixed micelle phase collected after full digestion (mouth, stomach, and small intestine) of the curcumin-excipient emulsion mixtures was measured. The concentration in the total raw digesta (before centrifugation) is a measure of the amount of curcumin that has not chemically degraded. The concentration in the mixed micelle phase (collected after centrifugation) is a measure of the amount of curcumin that is chemically stable and solubilized within the mixed micelles. These values were used to calculate the bioaccessibility of curcumin, i.e., the fraction of curcumin in the raw digesta that was solubilized within the mixed micelles. The size and electrical charge of the particles collected from the micelle phase was also measured.

The mixed micelles obtained from all three excipient emulsions contained highly negatively charged particles: ζ-potentials=−52±7, −53±6, and −37±6 mV for small, medium, and large droplets. The most likely reason for this high negative charge is that the mixed micelle phase contained colloidal particles that consisted primarily of anionic bile salts, phospholipids, and free fatty acids. The mean particle diameters of the mixed micelle phases collected from all three excipient emulsions were fairly similar (=167±19, 209±38 and 205±33 nm respectively), suggesting that they contained similar types of colloidal particles after digestion. The mixed micelle phases were collected from the raw digesta by centrifugation, so any larger particles would have been removed (such as non-digested oil droplets or calcium soaps).

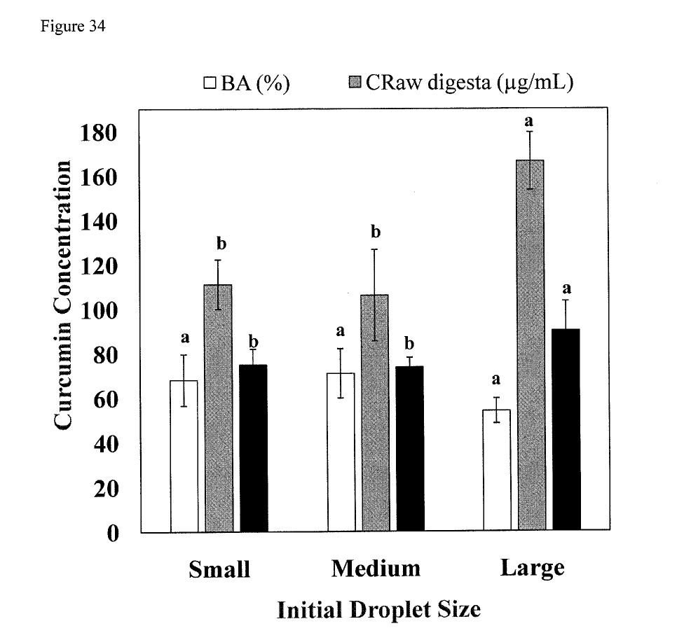

The actual amounts of curcumin present in the raw digesta and in the mixed micelle phase was significantly higher in the excipient emulsion containing large droplets, than in the ones containing small or medium droplets (FIG. 34). On the other hand, the bioavailability of curcumin was statistically similar in all three excipient emulsions. There are a number of possible physicochemical mechanisms that can account for these observations. The chemical degradation of curcumin is likely to be less rapid in the large droplets than in the small or medium droplets, because it has a lower specific surface area exposed to the surrounding aqueous phase. Consequently, a lower fraction of the curcumin is in direct contact with any catalysts present in the aqueous phase that could accelerate its degradation. On the other hand, the bioaccessibility of curcumin is likely to be higher in the mixed micelle phase formed from the smaller droplets because of the more rapid and extensive lipid digestion. The slower digestion of the large droplets may also have increased the chemical stability of curcumin because the curcumin molecules would spend more time inside the hydrophobic interiors of the droplets, rather than in the mixed micelles. The fact that the bioaccessibility of curcumin in all three emulsions was similar (FIG. 34), could have been because the final extent of digestion of the three emulsions was fairly similar. Consequently, there were similar amounts of mixed micelles available to solubilize the curcumin.

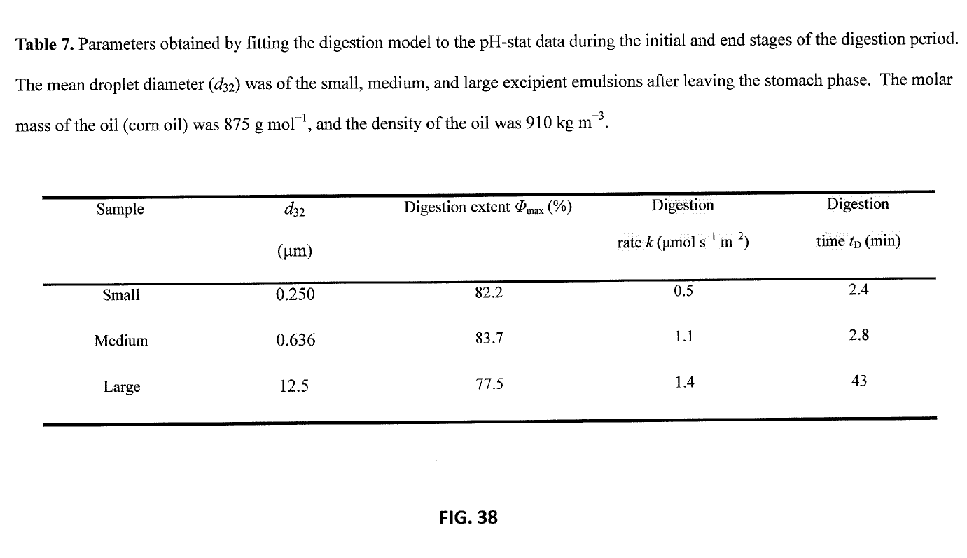

See also, Table 7 (FIG. 38) for parameters obtained by fitting the digestion model to the pH-stat data during the initial and end stages of the digestion period.

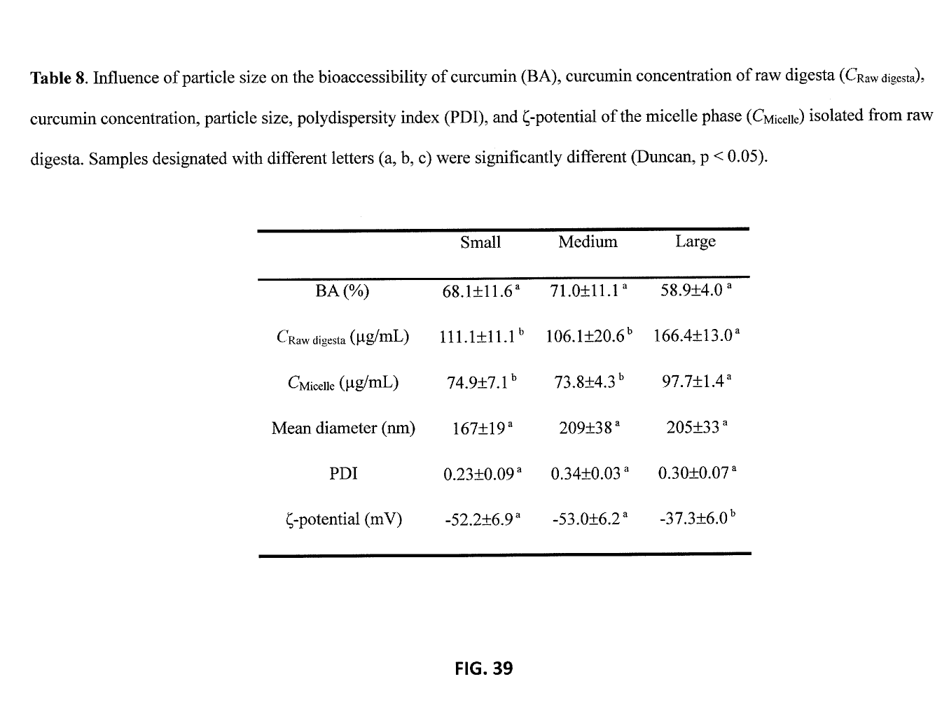

Additionally, see Table 8 (FIG. 39) for the influence of particle size on the bioaccessibility of curcumin after digestion.

Conclusions

In conclusion, this example shows that the effectiveness of excipient emulsions at increasing curcumin bioavailability depends on pre-ingestion incubation temperature and on lipid droplet size. The transfer of curcumin from the powdered form into the excipient emulsions was greater when they were incubated at 100° C. than at 30° C., presumably due to the increase in water-solubility and oil-solubility of curcumin with increasing temperature. The influence of droplet size on curcumin transfer was found to be more complex, which was attributed to the competing effects of droplet surface area on the chemical degradation and mass transfer rates. These effects have important implications for specifying optimum processing conditions and emulsion microstructures for enhancing curcumin bioavailability. Conditions should be optimized to ensure a high transfer of curcumin into the excipient emulsions, but to also ensure a low rate of curcumin degradation. Both of these effects increase with increasing temperature and decreasing droplet size, and therefore some compromise of processing conditions and emulsion microstructure is required.

The curcumin concentrations in the total digesta and mixed micelle phase generated by lipid digestion also depended on the initial droplet size of the excipient emulsions, decreasing in the following order: large>small≈medium. This effect was primarily attributed to the increased chemical stability of curcumin molecules encapsulated within large oil droplets (since the curcumin molecules would be further away from aqueous phase components that catalyse degradation). However, the bioaccessibility of curcumin was fairly independent of initial droplet size, which was attributed to the fact that the same amount of mixed micelles was formed at the end of the lipid digestion process for all three droplet sizes studied.

Although the invention has been described with respect to various embodiments, it should be realized these teachings are also capable of a wide variety of further and other embodiments within the spirit and scope of these teachings.

1-18. (canceled)

19. A method for improving oral bioavailability of pharmaceuticals or nutraceuticals, the method comprising:
designing a food matrix, the food matrix not having bioactivity above its normal nutritional function, that increases bioavailability of a predetermined pharmaceutical or nutraceutical by at least one of facilitating the release and solubilization of bioactive agents in the predetermined pharmaceutical or nutraceutical, altering the absorption of lipophilic bioactive agents in the predetermined pharmaceutical or nutraceutical, or interfering with chemical transformations that occur within gastrointestinal tract (GIT) or after absorption;
wherein the food matrix is co-ingested with the predetermined pharmaceutical or nutraceutical or being ingested at a specified time soon before or soon after the pharmaceutical or nutraceutical.

20. The method of claim 19 wherein the facilitating the release and solubilization of bioactive agents in the predetermined pharmaceutical or nutraceutical comprises at least one of enhancing breakdown of a matrix surrounding a bioactive agent, enhancing solubilization with a mixed micelle phase, altering mass transport processes within the GIT, or altering the motility of the GIT.

21. The method of claim 19 wherein the altering the absorption of lipophilic bioactive agents in the predetermined pharmaceutical or nutraceutical comprises at least one of increasing transport across a layer of epithelial cells surrounding the GIT or inhibiting the efflux mechanisms in membranes of intestinal epithelial cells.

22. The method of claim 19 further comprising:
using an in vitro GIT model or animal feeding study to verify improvement of oral bioavailability of the predetermined pharmaceutical or nutraceutical.

23. A composition comprising:
a food matrix, the food matrix not having bioactivity above its normal nutritional function, the food matrix being configured to increase bioavailability of a predetermined pharmaceutical or nutraceutical; and
the predetermined pharmaceutical or nutraceutical incorporated in the food matrix.

24. The composition of claim 23 wherein the food matrix is configured to increase bioavailability by configuring the food matrix to increase by at least one of facilitating the release and solubilization of bioactive agents in the predetermined pharmaceutical or nutraceutical, altering the absorption of lipophilic bioactive agents in the predetermined pharmaceutical or nutraceutical, or interfering with chemical transformations that occur within gastrointestinal tract (GIT) or after absorption.

25. The composition of claim 24 wherein release and solubilization of bioactive agents in the predetermined pharmaceutical or nutraceutical is facilitated by at least one of enhancing breakdown of a matrix surrounding a bioactive agent, enhancing solubilization with a mixed micelle phase, altering mass transport processes within the GIT, or altering the motility of the GIT.

26. The composition of claim 24 wherein absorption of lipophilic bioactive agents in the predetermined pharmaceutical or nutraceutical is altered by at least one of increasing transport across a layer of epithelial cells surrounding the GIT or inhibiting the efflux mechanisms in membranes of intestinal epithelial cells.

27. The composition of claim 23 wherein the predetermined nutraceutical is curcumin and the food matrix is one of an emulsion, oil, or a buffer solution.

28. The composition of claim 23 wherein the predetermined nutraceutical is β-carotene or α-carotene and the food matrix is one of an emulsion, oil, or a buffer solution.

29. The composition of claim 23 wherein the predetermined nutraceutical is coenzyme Q10 and the food matrix is one of an emulsion, oil, or a buffer solution.

30. The composition of claim 23 wherein the nutraceutical is long chain fatty acids and the food matrix is one of an emulsion, oil, or a buffer solution.
